(19) United States
(12) Patent Application Publication
(10) Pub. No.: US 2026/0157845 A1
(43) Pub. Date: Jun. 11, 2026

(54) FIXATION DEVICES, INSTRUMENTS, SYSTEMS AND METHODS OF USE

(71) Applicant: ARTHROTAK, LLC, Missoula, MT (US)

(72) Inventors: Amit NATHANI, Santa Barbara, CA (US); Donald E. RUNNING, Missoula, MT (US); Peter W. RUNNING, Missoula, MT (US); Dan JUSTIN, Orlando, FL (US); Matthew PIFER, Santa Barbara, CA (US); Geoffrey ABRAMS, Menlo Park, CA (US)

(73) Assignee: ARTHROTAK, LLC, Missoula, MT (US)

(21) Appl. No.: 19/538,525

(22) Filed: Feb. 12, 2026

Related U.S. Application Data
(63) Continuation-in-part of application No. PCT/US2025/050116, filed on Oct. 8, 2025.
(60) Provisional application No. 63/704,710, filed on Oct. 8, 2024.

(51) Int. Cl. A61F 2/08; A61F 2/00

(52) U.S. Cl. CPC A61F 2/0811; A61F 2002/0081; A61F 2002/0841; A61F 2002/0888; A61F 2220/0016

(57) ABSTRACT

The present disclosure is directed towards implants configured to compress a tendon in place relative to a bone, as well as associated systems, devices, instruments, and methods for performing tenotomy and tenodesis procedures utilizing the implants. The systems for compressing the bone include a sleeve having a first end, a second end, and a through-hole extending longitudinally through the sleeve from the first end to the second end, and a fixator having a sharp tip, the implant configured to be inserted through the through-hole. The implant includes a head, a stem extending from the head, and a cavity having a first opening in the head. The cavity extends through the head and into at least a portion of the stem, and a hard tip is connected to the stem.

### CROSS REFERENCE TO RELATED APPLICATION

This application is a continuation of PCT/US2025/050116, filed Oct. 8, 2025 and entitled FIXATION DEVICES, INSTRUMENTS, SYSTEMS AND METHODS OF USE, which claims priority benefit under 35 U.S.C. § 119(e) of U.S. Provisional Application No. 63/704,710 filed Oct. 8, 2024, which are incorporated herein by reference in their entireties.

### TECHNICAL FIELD

The present disclosure relates generally to surgeries, and more specifically, to surgeries involving soft tissue fixation. More specifically, but not exclusively, the present disclosure relates to devices, implants, instruments, systems, and methods for tenotomy and tenodesis procedures.

### BACKGROUND

Various procedures may be performed to repair soft tissue in the body. For example, tendons that attach muscle to bone or ligaments that attach bones to other bones may need to be repaired or replaced for various reasons. For example, an injury to the biceps tendon may require the biceps tendon to be tenotomized and subsequently fixed to the humeral bone. Alternatively, it may be desirable to attach one end of a tendon to a new fixation point to provide additional support to other tendons and muscles.

Currently available procedures for fixing soft tissue to a selected area on a bone include providing a suture through a selected portion of the soft tissue while securing the other end of the suture to a selected area on the bone. Various structures and methods, such as soft tissue suture anchors, can be provided to anchor or hold the suture in the selected bone area.

Typical soft tissue suture anchors may require the placement of the soft tissue suture anchor into the bone prior to the engagement of the soft tissue with the suture anchor. This procedure may be time consuming and require precise placement. Therefore, it may be desirable to provide a soft tissue attachment mechanism that may substantially simplify the reattachment or replacement of soft tissue during a surgical procedure.

A key aspect of biceps tenodesis procedures is achieving anatomic tensioning of the biceps tendon to the humeral bone. Proper anatomic tensioning after tenotomizing the tendon is not always achieved. If the tendon is over tightened, it will constrain the tendon and muscle and may lead to pain generation. If the tendon is under-tightened, it will lead to laxity in the tendon and muscle, a phenomenon known as a “Pop-eye” deformity. Therefore, it may be desirable to provide methods of soft tissue fixation that may make anatomic tensioning easier to achieve or to maintain during biceps tenodesis procedures.

Following surgery, a patient generally needs time to heal. While patients may be given additional treatment, such as medicine, to assist in the healing process, the patient's body may also supply healing agents to the surgical site. Therefore, it may be desirable to have a fixation device that provides access for biological healing agents contained within the intramedullary region of the bone to reach the region between the tendon and bone to aid in the healing process.

The presently relied on devices for soft tissue fixation include tenodesis screws (large implants requiring drilling), cortical buttons (surgical implants used in conjunction with sutures to compress the tendon to the bone via whip stitching), and suture anchors, none of which are configured to facilitate access for biological healing agents contained within the intramedullary region of the bone to reach the surgical site and aid in the healing process.

Thus, it is an object of the present disclosure to overcome one or more of the above-described drawbacks and/or disadvantages of the currently available devices and procedures.

### SUMMARY

The present disclosure is directed toward devices, implants, instruments, systems, and methods for tenotomy and tenodesis procedures.

The present disclosure provides, in a first aspect, an implant including a head, a stem extending from the head, and a cavity having a first opening in the head. The cavity extends through the head and into at least a portion of the stem, and a portal hole extends from an outer surface of the stem into the cavity.

The present disclosure provides, in a second aspect, a system for compressing a tendon to a bone including a sleeve having a first end, a second end opposite the first end, and a first through-hole extending longitudinally through the sleeve from the first end to the second end. The system further includes a fixator configured to be removably received within the first through-hole of the sleeve, the fixator having a sharp tip, and an implant configured to be inserted into the first through-hole of the sleeve to compress a tendon to the bone.

The present disclosure provides, in a third aspect, an implant sleeve including a tubular body having an inner cavity and a tip. The tip includes a first prong having a first side and a second side, a second prong having a first side and a second side, the second prong diametrically opposite to the first prong. The implant sleeve further includes a first arch diametrically opposite to a second arch, the first arch connected to the first side of the first prong and the first side of the second prong, and the second arch connected to the second side of the first prong and the second side of the second prong.

The present disclosure provides, in a fourth aspect, a k-wire cap or fixator cap including a body having a first end with a first opening of a first cavity and a second end with a second opening of a second cavity, the first end opposite the second end. The first cavity extends into the body toward the second end to a first depth, and the second cavity extends into the body toward the first end to a second depth, the second depth being further than the first depth.

The present disclosure provides, in a fifth aspect, a method for compressing a tendon to a bone including inserting a sleeve having a through-hole into a cut to hold the tendon in place relative to the bone, piercing a cavity in the bone to a first depth, inserting an implant into the cavity and impacting the implant to drive the implant into the cavity to compress the tendon to the bone, and removing the sleeve from the cut.

The present disclosure provides, in a sixth aspect, an implant including a head, a stem extending from the head, and a cavity having a first opening in the head. The cavity extends through the head and into at least a portion of the stem, and a hard tip is connected to the stem.

The present disclosure provides, in a seventh aspect, a method for compressing a tendon to a bone including pinning the tendon in place relative to the bone, inserting an implant into a cut to contact the tendon, impacting the implant to drive the implant into the bone to a first depth to compress the tendon to the bone, and removing the sleeve from the cut.

These and other objects, features, and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 depicts a first perspective view of a tack or implant, in accordance with an aspect of the present disclosure;

FIG. 2 depicts a second perspective view of the implant of FIG. 1, in accordance with an aspect of the present disclosure;

FIG. 3 depicts a top view of the implant of FIG. 1, in accordance with an aspect of the present disclosure;

FIG. 4 depicts a bottom view of the implant of FIG. 1, in accordance with an aspect of the present disclosure;

FIG. 5 depicts a first side view of the implant of FIG. 1, in accordance with an aspect of the present disclosure;

FIG. 6 depicts a second side view of the implant of FIG. 1, in accordance with an aspect of the present disclosure;

FIG. 7 depicts a third side view of the implant of FIG. 1, in accordance with an aspect of the present disclosure;

FIG. 8 depicts a fourth side view of the implant of FIG. 1, in accordance with an aspect of the present disclosure;

FIG. 9 depicts a first cross-sectional view of the implant of FIG. 1 taken along line 9-9 of FIG. 8, in accordance with an aspect of the present disclosure;

FIG. 10 depicts a second cross-sectional view of the implant of FIG. 1 taken along line 10-10 of FIG. 6, in accordance with an aspect of the present disclosure;

FIG. 11 depicts a perspective view of a sleeve, in accordance with an aspect of the present disclosure;

FIG. 12 depicts a close-up perspective view of a tip of the sleeve of FIG. 11, in accordance with an aspect of the present disclosure;

FIG. 13 depicts a first side view of the sleeve of FIG. 11, in accordance with an aspect of the present disclosure;

FIG. 14 depicts a second side view of the sleeve of FIG. 11, in accordance with an aspect of the present disclosure;

FIG. 15 depicts a first perspective view of a fixator or k-wire cap, in accordance with an aspect of the present disclosure;

FIG. 16 depicts a second perspective view of the k-wire cap of FIG. 15, in accordance with an aspect of the present disclosure;

FIG. 17 depicts a top view of the k-wire cap of FIG. 16, in accordance with an aspect of the present disclosure;

FIG. 18 depicts a bottom view of the k-wire cap of FIG. 16, in accordance with an aspect of the present disclosure;

FIG. 19 depicts a cross-sectional view of the k-wire cap of FIG. 16 taken along line 19-19 of FIG. 17, in accordance with an aspect of the present disclosure;

FIG. 20 depicts a side view of the sleeve of FIG. 11 and a sleeve plug being inserted into a cut to contact a tendon and/or a bone, in accordance with an aspect of the present disclosure;

FIG. 21 depicts a side view of the sleeve of FIG. 11 being used to secure the tendon to the bone, in accordance with an aspect of the present disclosure;

FIG. 22 depicts a side view of a punch and k-wire being inserted into/through the sleeve of FIG. 11, in accordance with an aspect of the present disclosure;

FIG. 23 depicts a side view of the k-wire cap of FIG. 16 being used to facilitate a first impact applied to the punch/k-wire to force the punch/k-wire into/through the bone, in accordance with an aspect of the present disclosure;

FIG. 24 depicts a side view of the k-wire cap of FIG. 16 being used to facilitate a second impact applied to the k-wire to force the k-wire into/through the bone, in accordance with an aspect of the present disclosure;

FIG. 25 depicts a side view of the punch being removed from the sleeve of FIG. 11, in accordance with an aspect of the present disclosure;

FIG. 26 depicts a side view of the sleeve of FIG. 11 and the k-wire ready to receive the implant of FIG. 1, in accordance with an aspect of the present disclosure;

FIG. 27 depicts a side view of an inserter being used to place the implant of FIG. 1 into/through the sleeve of FIG. 11 and into a bone, in accordance with an aspect of the present disclosure;

FIG. 28 depicts a side view the implant of FIG. 1 compressing the tendon to the bone, in accordance with an aspect of the present disclosure;

FIG. 29 depicts a cross-sectional side view of an alternative embodiment of the implant of FIG. 1 featuring a hard tip, in accordance with an aspect of the present disclosure;

FIG. 30 depicts a first perspective view of yet another an alternative embodiment of the implant of FIG. 1 in a first state and featuring a hard tip and expandable wings, in accordance with an aspect of the present disclosure;

FIG. 31 depicts a second perspective view of the implant of FIG. 30, in accordance with an aspect of the present disclosure;

FIG. 32 depicts a top view of the implant of FIG. 30, in accordance with an aspect of the present disclosure;

FIG. 33 depicts a bottom view of the implant of FIG. 30, in accordance with an aspect of the present disclosure;

FIG. 34 depicts a first side view of the implant of FIG. 30, in accordance with an aspect of the present disclosure;

FIG. 35 depicts a second side view of the implant of FIG. 30, in accordance with an aspect of the present disclosure;

FIG. 36 depicts a first cross-sectional view of the implant of FIG. 30 taken along line 36-36 of FIG. 35, in accordance with an aspect of the present disclosure;

FIG. 37 depicts a second cross-sectional view of the implant of FIG. 30 taken along line 37-37 of FIG. 34, in accordance with an aspect of the present disclosure;

FIG. 38 depicts a perspective view of an alternative embodiment of the inserter of FIG. 27, in accordance with an aspect of the present disclosure;

FIG. 39 depicts a bottom view of the inserter of FIG. 38, in accordance with an aspect of the present disclosure;

FIG. 40 depicts a top view of the inserter of FIG. 38, in accordance with an aspect of the present disclosure;

FIG. 41 depicts a side view of the inserter of FIG. 38, in accordance with an aspect of the present disclosure;

FIG. 42 depicts a cross-sectional view of the inserter of FIG. 38 taken along line 42 42 of FIG. 39, in accordance with an aspect of the present disclosure;

FIG. 43 depicts a close-up perspective view of a tip of the inserter of FIG. 38, in accordance with an aspect of the present disclosure;

FIG. 44 depicts a side view of the implant of FIG. 30 in a second state and engaged with the inserter of FIG. 38, in accordance with an aspect of the present disclosure;

FIG. 45 depicts a cross-sectional view of the implant of FIG. 30 in the second state and engaged with the inserter of FIG. 38, in accordance with an aspect of the present disclosure;

FIG. 46 depicts a cross-sectional view of the implant of FIG. 30 in the second state after being implanted into a bone, in accordance with an aspect of the present disclosure;

FIG. 47 depicts a cross-sectional side view of the inserter of FIG. 38 engaged with the implant of FIG. 30 in the first state, in accordance with an aspect of the present disclosure;

FIG. 48 depicts a cross-sectional side view of the implant of FIG. 30 engaged with the inserter of FIG. 38 being inserted through the sleeve of FIG. 11 and into a bone, in accordance with an aspect of the present disclosure;

FIG. 49 depicts a cross-sectional side view of an alternative embodiment of the implant of FIG. 30 having a cannula which extends through the hard tip, in accordance with an aspect of the present disclosure;

FIG. 50 depicts a side view of the inserter of FIG. 38 being used to place the implant of FIG. 49 through the sleeve of FIG. 11 and into a bone, in accordance with an aspect of the present disclosure;

FIG. 51 depicts a close-up perspective view of an alternative embodiment of the tip of the inserter of FIG. 38 having an opening of a channel, in accordance with an aspect of the present disclosure;

FIG. 52 depicts a cross-sectional side view of the inserter of FIG. 51, in accordance with an aspect of the present disclosure;

FIG. 53 depicts a perspective view of yet another an alternative embodiment of the implant of FIG. 1 featuring a suture passage on the head, in accordance with an aspect of the present disclosure;

FIG. 54 depicts a top-down view of the implant of FIG. 53, in accordance with an aspect of the present disclosure;

FIG. 55 depicts a first side view of the implant of FIG. 53, in accordance with an aspect of the present disclosure;

FIG. 56 depicts a second side view of the implant of FIG. 53 being used with a suture, in accordance with an aspect of the present disclosure;

FIG. 57 depicts a perspective view of yet another alternative embodiment of the implant of FIG. 1 featuring a suture-passage instead of the head, in accordance with an aspect of the present disclosure;

FIG. 58 depicts a top-down view of the implant of FIG. 57, in accordance with an aspect of the present disclosure;

FIG. 59 depicts a first side view of the implant of FIG. 57, in accordance with an aspect of the present disclosure;

FIG. 60 depicts a second side view of the implant of FIG. 57 being used with a suture, in accordance with an aspect of the present disclosure;

FIG. 61 depicts a perspective view of an alternative embodiment of the inserter of FIG. 38, in accordance with an aspect of the present disclosure;

FIG. 62 depicts a bottom view of the inserter of FIG. 61, in accordance with an aspect of the present disclosure;

FIG. 63 depicts a top view of the inserter of FIG. 61, in accordance with an aspect of the present disclosure;

FIG. 64 depicts a side view of the inserter of FIG. 61, in accordance with an aspect of the present disclosure;

FIG. 65 depicts a cross-sectional view of the inserter of FIG. 61 taken along line 65 65 of FIG. 62, in accordance with an aspect of the present disclosure;

FIG. 66 depicts a perspective view of an alternative embodiment of the hard tip of FIG. 29, in accordance with an aspect of the present disclosure;

FIG. 67 depicts a side view of the hard tip of FIG. 66, in accordance with an aspect of the present disclosure;

FIG. 68 depicts a perspective view of the inserter of FIG. 61 engaged with an alternative embodiment of the implant of FIG. 1 that includes the hard tip of FIG. 66, in accordance with an aspect of the present disclosure;

FIG. 69 depicts a side view of a portion of the inserter of FIG. 61 engaged with the implant of FIG. 68 and the hard tip of FIG. 66, in accordance with an aspect of the present disclosure;

FIG. 70 depicts a cross-sectional view of the inserter of FIG. 61 engaged with the implant of FIG. 68 and the hard tip of FIG. 66 along line 70-70 of FIG. 69, in accordance with an aspect of the present disclosure;

FIG. 71 depicts a first perspective view of yet another an alternative embodiment of the implant of FIG. 1 that features a modular head and a modular stem, in accordance with an aspect of the present disclosure;

FIG. 72 depicts a second perspective view of the implant of FIG. 71, in accordance with an aspect of the present disclosure;

FIG. 73 depicts an exploded view of the implant of FIG. 71, in accordance with an aspect of the present disclosure;

FIG. 74 depicts a first perspective view of the modular stem of the implant of FIG. 71, in accordance with an aspect of the present disclosure;

FIG. 75 depicts a second perspective view of the modular stem of FIG. 74, in accordance with an aspect of the present disclosure;

FIG. 76 depicts a first perspective view of the modular head of the implant of FIG. 71, in accordance with an aspect of the present disclosure;

FIG. 77 depicts a second perspective view of the modular head of FIG. 76, in accordance with an aspect of the present disclosure;

FIG. 78 depicts a top view of the modular head of FIG. 76, in accordance with an aspect of the present disclosure;

FIG. 79 depicts a first perspective view of an alternative embodiment of the modular stem of FIG. 74 that lacks an anti-rotation feature, in accordance with an aspect of the present disclosure;

FIG. 80 depicts a second perspective view of the modular stem of FIG. 79, in accordance with an aspect of the present disclosure;

FIG. 81 depicts a first perspective view of an alternative embodiment of the modular head of FIG. 76 that lacks an anti-rotation feature, in accordance with an aspect of the present disclosure;

FIG. 82 depicts a second perspective view of the modular head of FIG. 81, in accordance with an aspect of the present disclosure;

FIG. 83 depicts a top view of the modular head of FIG. 81, in accordance with an aspect of the present disclosure;

FIG. 84 depicts a first perspective view of an alternative embodiment of the modular head of FIG. 81 having a circular shape, in accordance with an aspect of the present disclosure;

FIG. 85 depicts a second perspective view of the modular head of FIG. 84, in accordance with an aspect of the present disclosure;

FIG. 86 depicts a top view of the modular head of FIG. 84, in accordance with an aspect of the present disclosure;

FIG. 87 depicts a first perspective view of yet another alternative embodiment of the modular head of FIG. 81 having a substantially quadrilateral shape, in accordance with an aspect of the present disclosure;

FIG. 88 depicts a second perspective view of the modular head of FIG. 87, in accordance with an aspect of the present disclosure;

FIG. 89 depicts a top view of the modular head of FIG. 87, in accordance with an aspect of the present disclosure;

FIG. 90 depicts a first perspective view of yet another alternative embodiment of the modular head of FIG. 81 having a triangular shape, in accordance with an aspect of the present disclosure;

FIG. 91 depicts a second perspective view of the modular head of FIG. 90, in accordance with an aspect of the present disclosure;

FIG. 92 depicts a top view of the modular head of FIG. 90, in accordance with an aspect of the present disclosure;

FIG. 93 depicts a first perspective view of a plate for use with at least the stem of FIG. 79, in accordance with an aspect of the present disclosure;

FIG. 94 depicts a second perspective view of the plate of FIG. 93, in accordance with an aspect of the present disclosure;

FIG. 95 depicts a first perspective view of the plate of FIG. 93 engaged with multiple stems, each stem of the multiple stems being identical to the stem of FIG. 79, in accordance with an aspect of the present disclosure; and

FIG. 96 depicts a second perspective view of the plate of FIG. 93 engaged with multiple stems, each stem of the multiple stems being identical to the stem of FIG. 79, in accordance with an aspect of the present disclosure.

### DETAILED DESCRIPTION

Generally stated, disclosed herein are devices, implants, instruments, and systems for tenotomy and tenodesis procedures. Further, methods for using the devices, implants, instruments, and systems for tenotomy and tenodesis procedures are discussed.

In this detailed description and the following claims, the words proximal, distal, anterior, posterior, medial, lateral, superior and inferior are defined by their standard usage for indicating a particular part or portion of a bone, instrument, or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

Similarly, positions or directions may be used herein with reference to anatomical structures or surfaces. For example, as the current devices, implants, systems, instrumentation and methods are described herein with reference to use with the bones of the arm and shoulder, the bones of the arm, shoulder and upper arm may be used to describe the surfaces, positions, directions or orientations of the devices, implants, systems, instrumentation and methods. Further, the devices, systems, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the devices, implants, systems, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the disclosure. For example, the devices, implants, systems, instrumentation and methods, and the aspects, components, features and the like thereof, described herein with respect to the right arm may be mirrored so that they likewise function with the left arm. Further, the devices, implants, systems, instrumentation and methods, and the aspects, components, features and the like thereof, disclosed herein are described with respect to the arm for brevity purposes, but it should be understood that the devices, implants, systems, instrumentation and methods may be used with other bones of the body having similar structures.

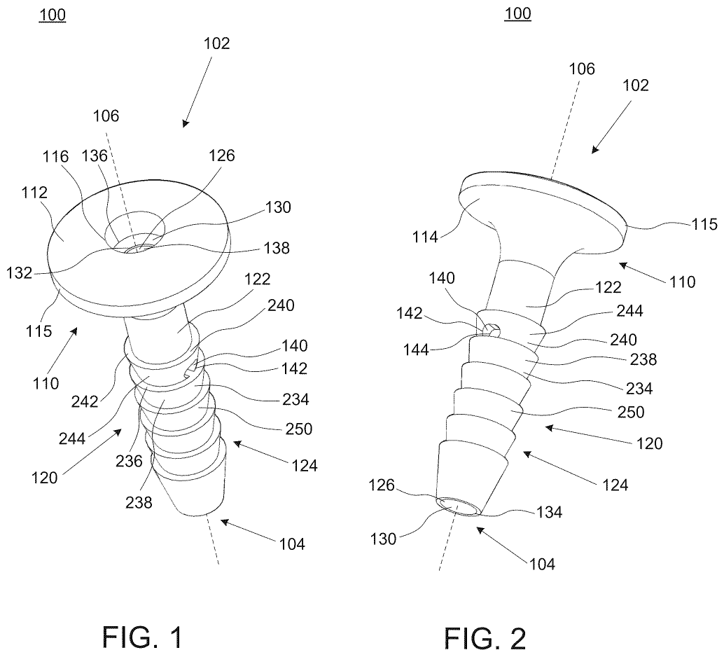

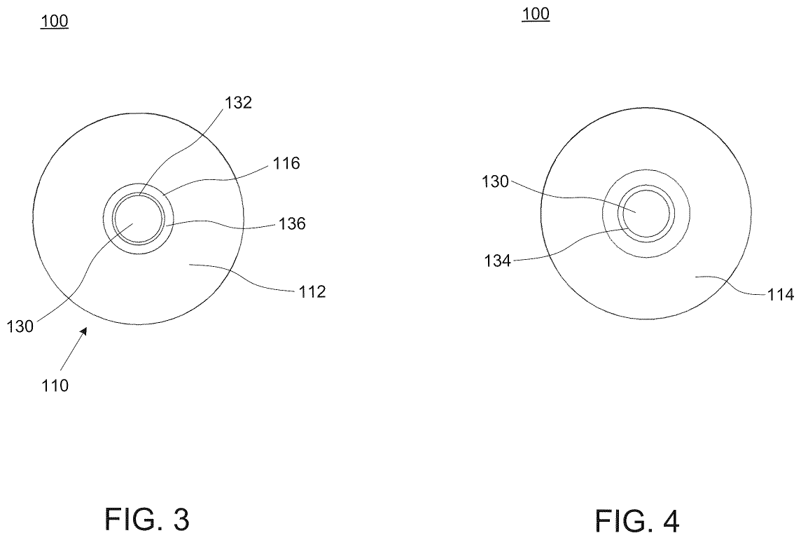

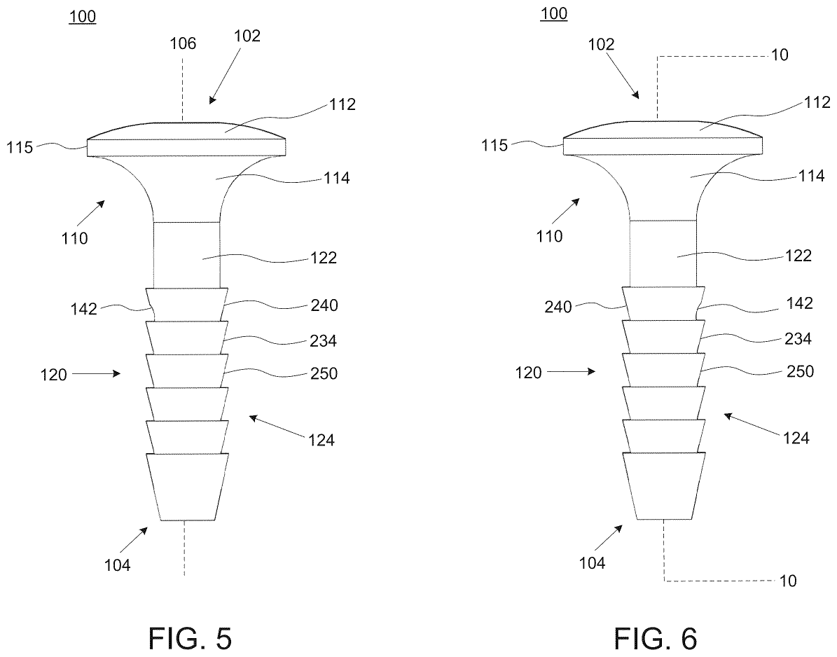

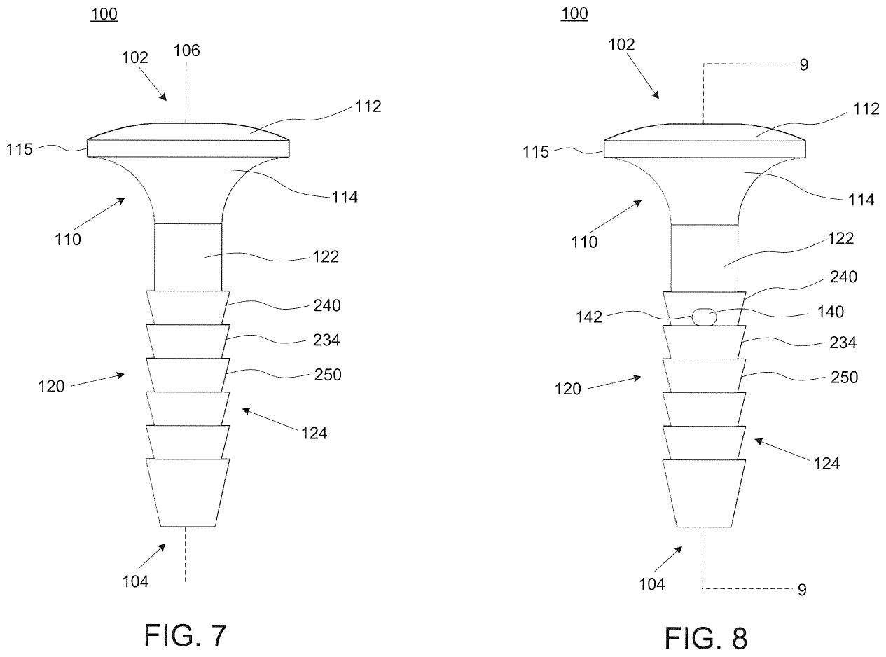

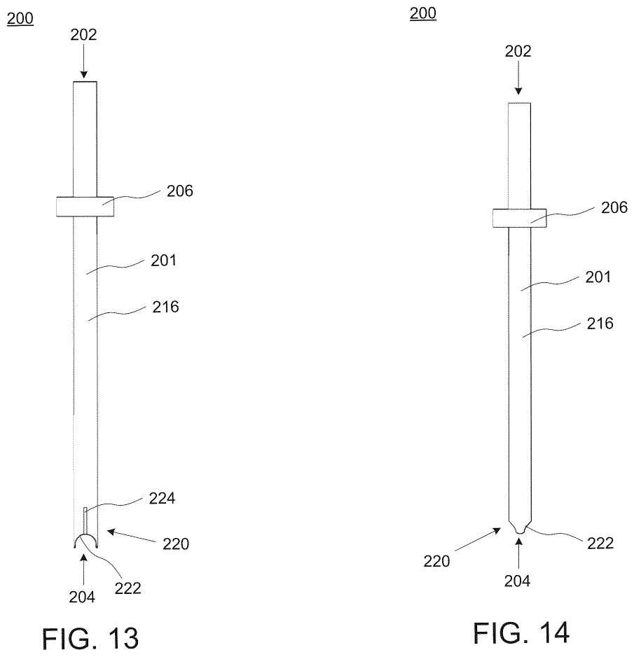

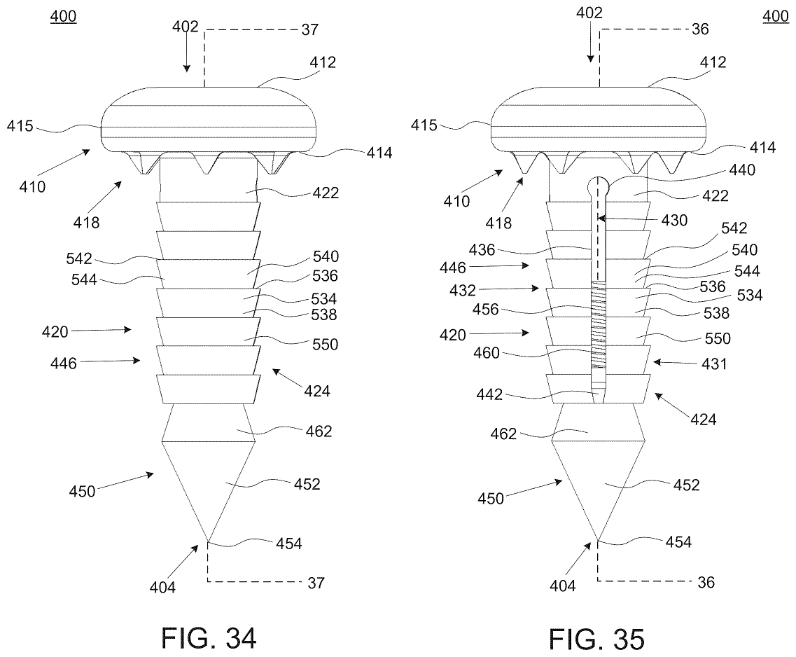

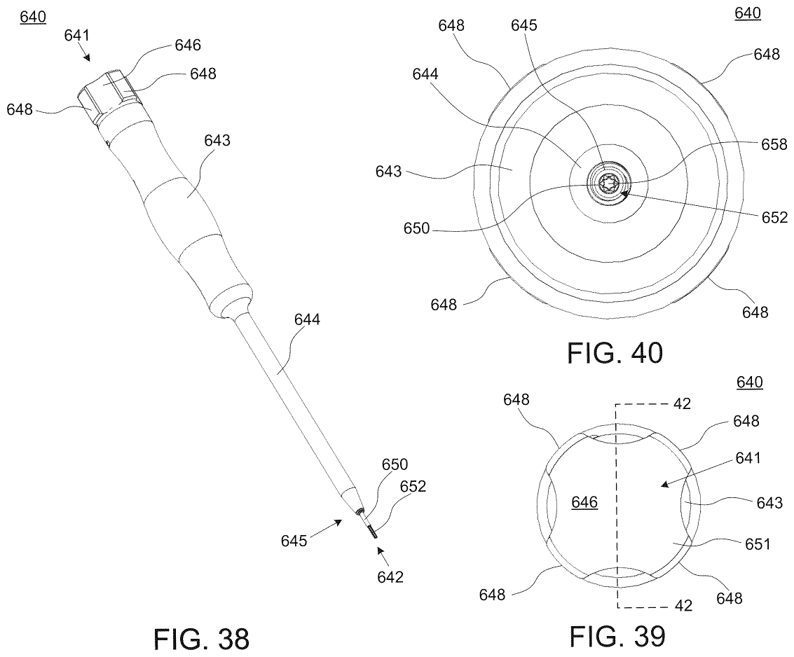

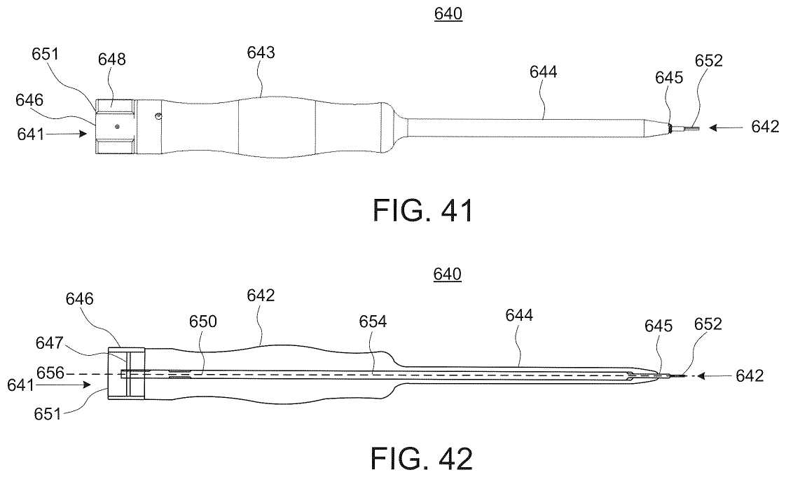

Referring to the drawings, wherein like reference numerals are used to indicate like or analogous components throughout the several views, and with particular reference to FIGS. 1-52, instruments, devices, implants, systems, and methods of using the instruments, devices, implants, and systems for tenotomy and tenodesis procedures are shown. Specifically, the procedures may include soft tissue fixation and/or compression, including biologically enhanced soft tissue fixation devices.

As described in more detail below and shown in FIGS. 1-29, a system for soft tissue fixation (i.e., compressing or squishing a soft tissue to a pre-selected bone/bony structure (e.g., a cortical bone)) may include a tack or implant 100, a sleeve 200, a sleeve plug 214, a fixator or k-wire 320, a k-wire cap or fixator cap 300, a punch 322, and/or an inserter 340.

As shown in FIGS. 1-10 and 29, the implant 100 for soft tissue fixation may have a first end 102 and a second end 104 with a head 110 connected to a stem 120. The stem 120 may extend away from a bottom surface 114 of the head 110 to the second end 104 of the implant 100. The implant 100 may be, for example, a singular, integral, or monolithic piece (i.e., of one-piece construction), or may be formed from a plurality of components that are coupled (e.g., rigidly coupled) together to form the implant 100. For example, in some embodiments, the head 110 and the stem 120 may be monolithically formed, whereas in other embodiments the head 110 and the stem 120 may be modular to allow various differently shaped components to be combined as needed. The head 110 and the stem 120 may be provided to fully secure a soft tissue (e.g., a tendon) in or to the bone. The implant 100 may be made by machine or injection molding, or it may be 3D printed, among other processes. In an example, the implant 100 may be made of PEEK, stainless metal, ultra-high molecular weight polyethylene, or a resorbable material, or a combination of the foregoing. For example, in some embodiments, the head 110 may be made of a different material than the stem 120.

With continued reference to FIGS. 1-10 and 29, the head 110 may be substantially circular and may further include a top surface 112 opposite to the bottom surface 114. In some embodiments, the head 110 may be a different shape, such as ovular. In further embodiments, the head 110 may be some non-rounded polygonal shape. The top surface 112 may be connected to the bottom surface 114 by a side surface 115. The head 110 may also include a first opening 116 of a through-bore or cannula 130, and the first opening 116 may be located in or on the top surface 112. The bottom surface 114 may be configured to compress a soft tissue (e.g., a tendon) to the bone when the implant 100 is inserted into a bone, such that the tendon may be fixed in place relative to the bone, as described in greater detail below.

As shown in FIGS. 1-2 and 7-10, the stem 120 may include an exterior or outer surface 122 and an interior or inner surface 126. The inner surface 126 may extend along the cannula 130 on the interior of the stem 120. The stem 120 may also include a plurality of teeth or ridges 124 positioned along the outer surface 122 of the stem 120. The stem 120 may further include at least one vent or portal hole 140 extending from the cannula 130 to the outer surface 122 of the stem 120 of the implant 100, as shown in FIGS. 1-2 and 8-10. In some embodiments (not shown), there may be a plurality of portal holes along the length of the stem 120 and/or optionally circumferentially positioned around the stem 120. The at least one portal hole 140 may allow for or permit the passage of biological agents between the cannula 130 of the implant 100 and the bone that the implant 100 is inserted into. When inserted, the stem 120 of the implant 100 may be positioned at least partially within the intramedullary canal of the bone.

With continued references to FIGS. 1-2 and 5-10, the ridges 124 may be disposed on the outer surface 122 of the stem 120 toward the second end 104 of the implant 100. The ridges 124 may be circumferentially continuous along the outer surface 122 (i.e., around an exterior of the stem 120). Although not shown, the ridges 124 may alternatively extend circumferentially around the outer surface 122 of the stem 120, but the ridges 124 may not be continuous as the ridges 124 extend circumferentially around the stem 120. For example, the ridges 124 may extend from the second end 104 toward the first end 102 along a first portion of the outer surface 122 of the stem 120. A second portion of the stem 120 or the remainder of the outer surface 122 of the stem 120 may be, for example, circumferentially smooth. The second portion of the stem 120 may contact or be coupled to the head 110. The placement and/or orientation of the ridges 124 toward the second end 104 of the implant 100 may allow for the ridges 124 to engage the bone to assist in fully securing the implant 100 to a bone.

A first ridge 234 of the ridges 124 may have a first upper surface 236 which may be substantially flat or planar and which may substantially face the first end 102, and a first lower surface 238 which may be inclined, tapered or angled, as shown in FIGS. 1-2 and 5-8. The first lower surface 238 may be inclined, tapered, or angled, for example, in a direction from the first end 102 toward the second end 104. The upper surface 236 may contact or be coupled to a second ridge 240 of the ridges 124, and the lower surface 238 may contact or be coupled to a third ridge 250 of the ridges 124. In some embodiments, each ridge of the ridges 124 may be longitudinally aligned along a first longitudinal axis 106. The ridges 124 (e.g., the upper surface and/or the tapering of the incline described above) may be shaped, for example, to permit the ridges 124 to act as barbs or fasteners to engage the bone when the implant 100 is inserted to hold the implant 100 in place relative to the bone. As the surgical site heals, the bone may heal around the ridges 124 to further secure the implant 100 (and the tendon) in place relative to the bone. Although the upper surfaces 236, 242 of the ridges 234, 240 are shown as extending from the outer surface 122 of the stem 120 the same distance, it is also contemplated that the upper surfaces 236, 240 along the length of the stem 120 may extend away from the outer surface 122 of the stem 120 different distances. In one embodiment, the upper surfaces 236, 242 may have different widths or circumferences that taper along the length of the stem 120 with the larger width or circumference being closest to the head 110 or first end 102 of the implant 100.

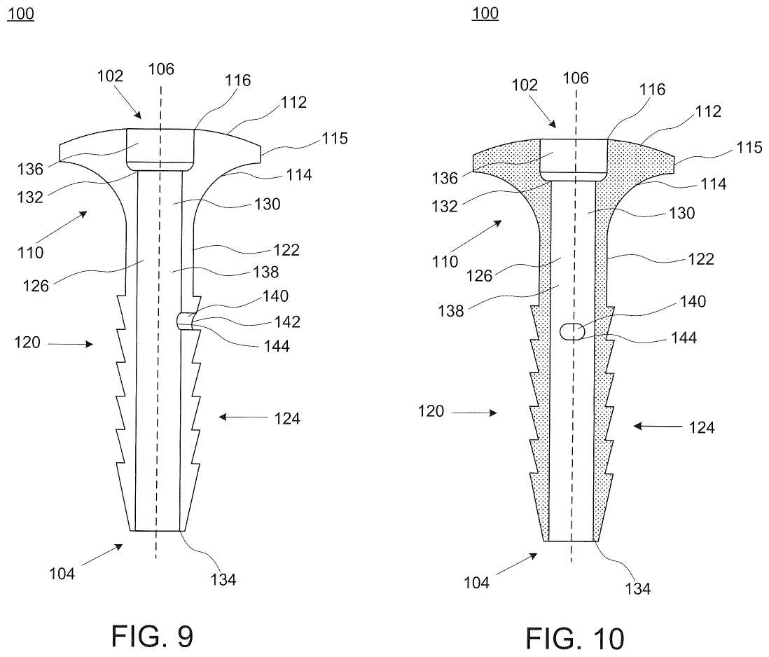

The inner surface 126 may bound by at least a portion of the cannula 130, as shown in FIGS. 9-10 and 29. The cannula 130 may extend from the first opening 116 through the head 110 and through at least a portion of the stem 120 to permit delivery or passage of biological agents. In some embodiments, the cannula 130 may be a partial cannula and may extend through only a portion of the stem 120, as shown in FIG. 29. In other embodiments, the cannula 130 may extend completely through the head 110 and the stem 120, for example, along the first longitudinal axis 106 of the implant, as shown in FIGS. 1-10.

As shown in FIGS. 9-10 and 29, the cannula 130 may include a counter-bore 136 and a coaxial bore 138. The counter-bore 136 may extend from the first opening 116 to a second opening 132. The coaxial bore 138 may extend from the second opening 132 toward the second end 104. In some embodiments, the coaxial bore 138 may extend to a third opening 134 located at or near the second end 104. When the implant 100 is inserted into the bone, the third opening 134 may contact and/or be in fluid communication with an intramedullary canal of a patient. In other embodiments, the coaxial bore 138 may extend to a hard tip 128 located at or near the second end 104, as shown in FIG. 29. In such an embodiment, the hard tip 128 may be pre-molded into the implant 100, for example, the implant 100 may be formed around the hard tip 128 or the hard tip 128 may be inserted into the implant 100 during formation. The first opening 116 and/or the counter-bore 136 may have a diameter which is larger than a diameter of the second opening 132, the third opening 134, and/or the coaxial bore 138.

The first opening 116, counter-bore 136, second opening 132, coaxial bore 138, and/or the third opening 134 (i.e., the various structures comprising the cannula) may be in fluid communication with each other, the portal hole 140, and/or an exterior of the implant 100 to permit various instruments to be placed in the cannula 130 and to permit the passage or flow of biological agents through the cannula 130 and/or the portal hole 140 after the implant 100 has been inserted into a bone.

In an embodiment, the portal hole 140 may be located within a ridge (e.g., the first ridge 234 or the second ridge 240) of the ridges 124. The portal hole 140 may have, for example, a flat or planar bottom surface and may engage or contact a portion of the first upper surface 236 of the first ridge 234, wherein the remainder of the portal hole 140 extends from the first upper surface 236 through a portion of the second lower surface 244 of the second ridge 240 towards the first end 102. The portal hole 140 may have an external opening 142 on the outer surface 122 and an internal opening 144 on the inner surface 126, as shown in FIG. 9. A passageway may extend from the external opening 142 through the outer surface 122 to the internal opening 144 of the inner surface 126.

The internal opening 144 may be in fluid communication with the cannula 130, and the external opening 142 may be in fluid communication with a space/area between the tendon and the bone when the implant 100 is inserted into a bone. The portal hole 140 may therefore be positioned and/or configured (e.g., sized, shaped and/or dimensioned) such that, when the implant 100 is inserted into a bone to pin the tendon thereto, the internal opening 144 may be in fluid communication with the intramedullary canal (e.g., via the cannula 130 and the third opening 134), and the external opening 142 may be positioned longitudinally along the outer surface 122 such that it is in fluid communication with the space or area between the tendon and the bone. Such fluid communication may permit transportation or delivery of a patient's natural biological healing agents from within the intramedullary canal to enter the cannula 130 through the third opening 134 and then travel within the cannula 130 (e.g., the coaxial bore 138) into the internal opening 144 of the portal hole 140, and then subsequently out of the external opening 142 to access the space and/or area between the compressed tendon and the bone, promoting healing of surgical tissues (e.g., the bone and the tendon).

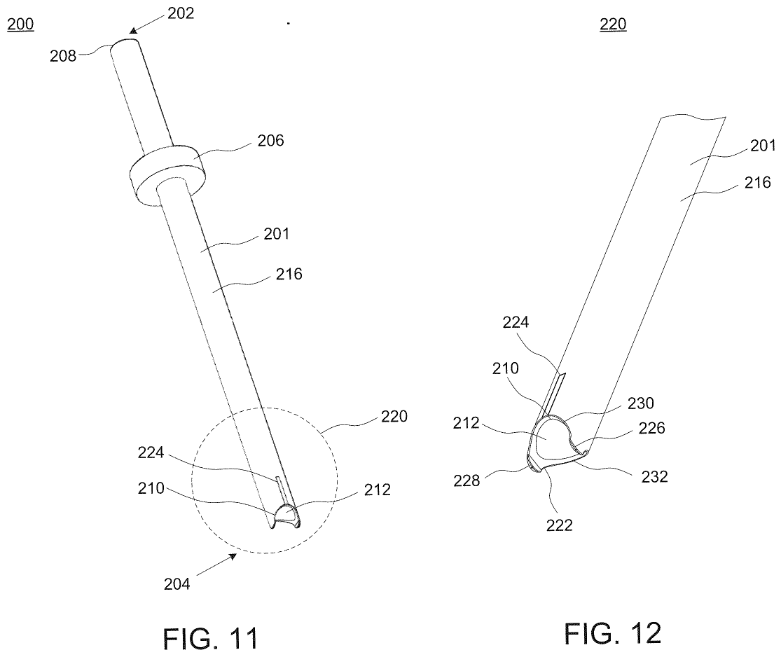

Turning to FIGS. 11-14, the system may include a sleeve 200 configured (e.g., sized, shaped and/or dimensioned) to be inserted into a surgical incision, cut, or wound. In embodiments, the sleeve 200 may further be configured to be inserted into a grommet or surgical dam (not shown), which may also be inserted into the cut. The sleeve 200 may further be configured (e.g., sized, shaped and/or dimensioned) to facilitate the insertion of various components, including the implant 100, into the surgical incision, cut, or wound and/or the bone. The sleeve 200 may include a body 201, a first end 202 having a first opening 208, and a second end 204 having a second opening 210. In some embodiments, the body 201 may be tubular. A through-bore or channel 212 may extend through the body 201 from the first opening 208 to the second opening 210, such that the first opening 208 is in fluid communication with the second opening 210. The first opening 208, the second opening 210, and/or the channel 212 may have a diameter which is larger than a largest width or a diameter of the implant 100 to permit the implant 100 to fit through (e.g., to be inserted into and/or through) the first opening 208 and the channel 212, and to exit the second opening 210 (e.g., out of the channel 212), as described in greater detail below with respect to the method of the present disclosure. More specifically, the head 110 of the implant 100 may have a diameter that is smaller than the diameter of the first opening 208, the second opening 210, and the channel 212. At least a portion of the body 201 may be transparent to assist with visualization of the implant 100 and various other instruments described herein as they pass into and/or through the channel 212. The transparent portion of the body 201 may be, for example, at least one window (not shown) along the length of the sleeve 200.

The sleeve 200 may include a collar 206 extending longitudinally along a portion of the body 201 and circumferentially around an outer surface 216 of the body 201. The collar 206 may permit a surgeon to ergonomically control the sleeve 200 during insertion into and removal from the cut or incision, as described in more detail below. In an example depicted in FIGS. 11-14, the collar 206 may be disposed towards the first end 202 of the sleeve 200, for example, between a midpoint of the sleeve 200 and the first end 202.

The second end 204 of the sleeve 200 may further include a sleeve tip 220 configured (e.g., sized, shaped and/or dimensioned) to hold a tendon in place relative to a bone during the process of inserting the implant 100 into the incision and/or the bone. The tip 220 may include the second opening 210. An edge 222 of the second opening 210 may include a plurality of prongs 226, 228 and a plurality of arches 230, 232. In an example shown in FIGS. 11-12, the plurality of prongs 226, 228 may include a first prong 226 and a second prong 228 positioned opposite from each other along the edge 222. The plurality of arches 230, 232 may include a first arch 230 and a second arch 232 positioned opposite from each other along the edge 222. The first arch 230 may connect the first prong 226 to the second prong 228 on a first side and the second arch 232 may connect the first prong 226 to the second prong 228 on a second side. Although the example embodiment shows the two prongs 226, 228 and the two arches 230, 232, alternative numbers of prongs and arche are also expressly contemplated.

The plurality of prongs 226, 228 may contact and/or engage a tendon to pin and/or secure the tendon in place relative to a bone when the sleeve 200 is inserted into the cut or incision to contact at least a portion of the bone. Importantly, the prongs 226, 228 may be configured (e.g., sized, shaped and/or dimensioned) to contact the tendon and hold the tendon in place such that anatomic tensioning of the tendon (e.g., the biceps tendon) is maintained throughout the tenodesis procedure (i.e., during insertion of the implant 100 into the bone).

Maintaining natural anatomic tensioning of the tendon may obviate the need for a surgeon to tenotomize and then manually re-tension the tendon, which may in turn reduce and/or eliminate the risk of negative surgical outcomes (e.g., pop-eye deformity).

The sleeve tip 220 may further include a measuring line or alignment indicator 224 located at a crown or a center of the first arch 230 and/or the second arch 232, as shown in FIGS. 11-13. The alignment indicator 224 permits a surgeon or other medical professional to visualize various aspects of the procedure, as described further below. For example, the alignment indicator 224 may be used to determine whether certain components (e.g., the implant 100, the punch 322, the k-wire 320, etc.) are aligned in the desired position over the soft tissue to be fixed to the bone. The alignment indicator 224 indicates or otherwise provides the central position of the implant 100, punch 322 and k-wire 320 as they pass through the sleeve 200 allowing for the user to visualize the engagement position with the soft tissue and bone. The alignment indicator 224 may further provide an indication of a rotational alignment of the sleeve 200 relative to the implant 100, which may be valuable, for example, in embodiments where the head 110 of the implant 100 is a shape other than circular (e.g., ovular or polygonal), in which case the alignment of the head 110 (and thus, the implant 100 as a whole) may be of increased importance.

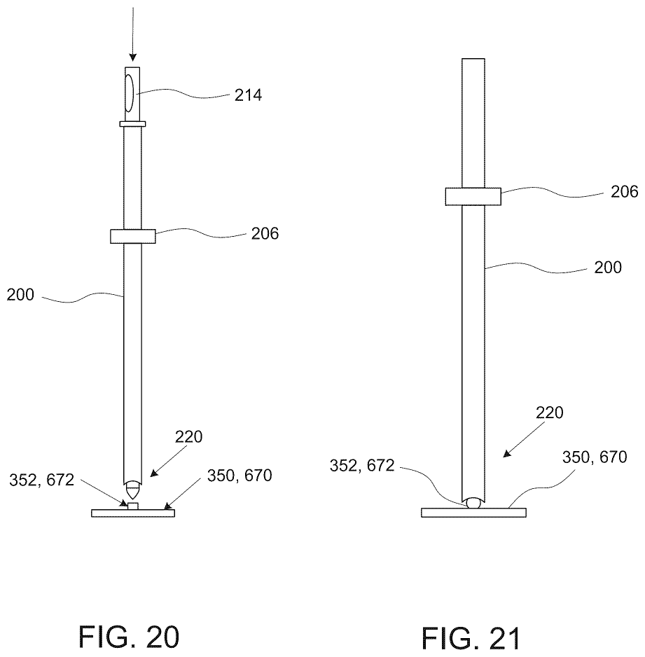

The sleeve 200 may be configured to removably receive the sleeve plug 214 in the channel 212, as shown in FIG. 20. When the sleeve 200 is initially inserted into the surgical cut, the channel 212 may be occupied or substantially occupied by the sleeve plug 214 such that fluid communication between the first opening 208 and the second opening 210 of the sleeve 200 may be prevented, blocked, reduced, or otherwise obstructed. The presence of the sleeve plug 214 within the channel 212 may prevent unwanted matter from clogging and/or contaminating the channel 212 when the sleeve 200 is inserted into the surgical cut. The sleeve plug 214 may then be removed from the sleeve 200 to expose the channel 212 and establish or reestablish fluid communication between the first opening 208 and the second opening 210 of the sleeve 200. Other components, including the implant 100, as described in more detail below, may then be inserted into and/or through the channel 212 (e.g., into and/or out of the first opening 208 and/or the second opening 210).

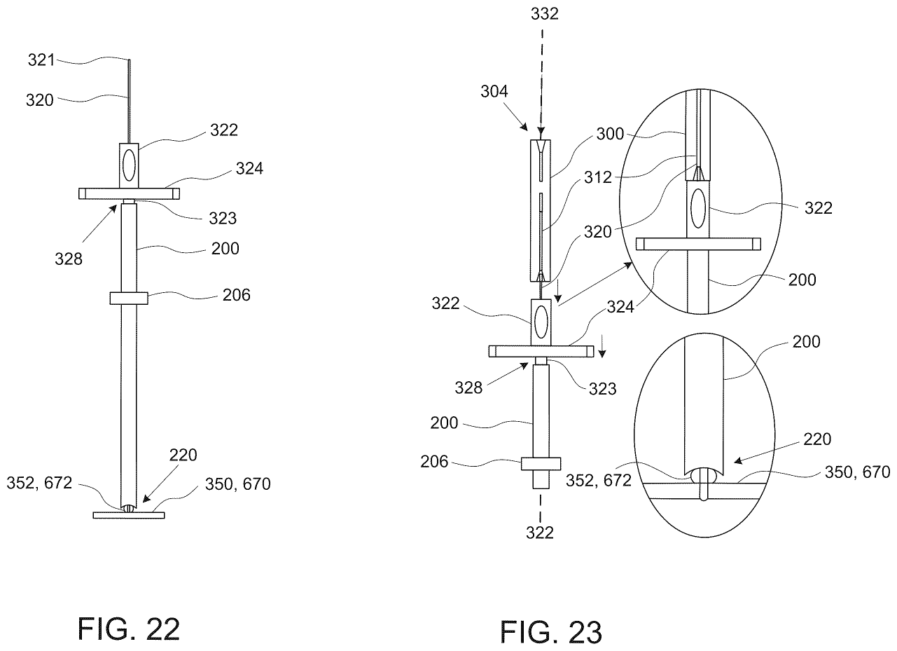

One such instrument which may be inserted into the channel 212 of the sleeve 200 during the procedure is the punch 322, as shown in FIGS. 22-25. The punch 322 may include a handle 324 and a first portion 323 extending from the handle 324 to a punch tip 326, as shown in FIG. 23. The handle 324 functions as a gripping point for the punch 322 when the punch 322 is being inserted into the channel 212, and further allows for the punch 322 to be back-tapped out of the surgical cut and/or a bone 350, as shown in FIG. 25. The punch tip 326 may be made of a hard material, such as metal, to permit the punch tip 326 to be driven into the bone 350 to a first predetermined depth in response to a first impact applied (directly or indirectly) to the punch 322. The punch 322 may also be configured to receive the k-wire 320, wherein the punch tip 326 may be hollow and include a channel to permit the k-wire 320 to extend beyond the punch tip 326 and into the bone 350 to a second predetermined depth in response to a second impact. The first impact and the second impact are described in more detail below with respect to the method of the present disclosure.

The first portion 323 of the punch 322 may have a diameter which is smaller than the diameter of the first opening 208, the second opening 210, and/or the channel 212 to permit at least the first portion and the tip 326 to be inserted into the channel 212 as shown in FIGS. 22-23 and 25. When the punch 322 is inserted into the channel 212 of the sleeve 200, the punch tip 326 may extend beyond (e.g., exit) the second opening 210 of the sleeve 200 to contact the soft tissue, for example, a tendon 352, and/or the bone 350. The first portion 323 may have a predetermined length which is longer than a length of the sleeve 200 from the first end 202 to the second end 204, such that a first gap 328 is left between the first end 202 of the sleeve 200 and the handle 324 of the punch 322 when the punch 322 is inserted into the channel 212 of the sleeve 200, as can be seen in FIGS. 22-23.

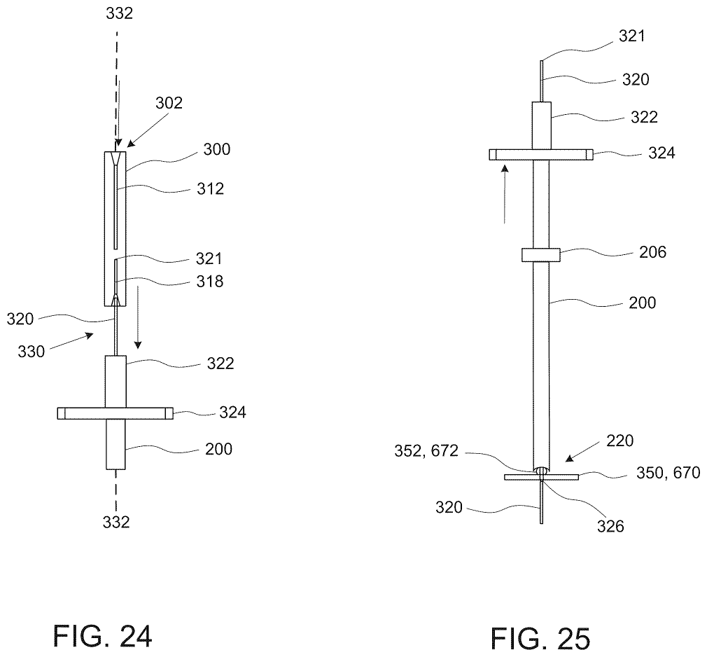

The first gap 328 between the handle 324 of the punch 322 and the first end 202 of the sleeve 200 may be a distance and/or length which is equal to the first predetermined depth to act as a stop for the punch tip 326 preventing the punch tip 326 from being driven undesirably into the bone 350 to a depth which is further than the first predetermined depth. For example, the first impact applied to the punch 322 may drive the first portion 323 further into the channel 212 such that the punch handle 324 contacts and/or engages the first end 202 of the sleeve 200, causing the punch tip 326 to puncture through the tendon 352 and/or into the bone 350 to the first predetermined depth, as shown in FIGS. 23-24. Once the punch tip 326 is driven through the tendon 352 and/or into the bone 350 to the first predetermined depth, the handle 324 may then be back-tapped to remove the punch tip 326 from the tendon 352 and/or the bone 350 (and/or to remove the punch 322 from the channel 212). Back-tapping may include tapping, hammering or otherwise impacting the punch 322 (e.g., the handle 324) in a direction opposite of inserting the punch 322 into the channel 212. Removing the punch 322, and therefore also the punch tip 326, from the channel 212 may leave a cavity in the bone 350 which is configured to receive the implant 100.

In some embodiments where a patient's bone is harder than an average and/or expected hardness, the implant 100 may feature the hard tip 128 shown in FIG. 29. The hard tip 128 may be utilized to pierce the bone 350 instead of the punch 322, punch tip 326 and/or the k-wire 320, as described in greater detail below. The hard tip 128 may be made of a material, such as metal, that is hard enough to pierce hard bone in the absence of the punch 322 and/or the k-wire 320.

The first impact may be applied to the punch 322 and the k-wire 320, for example directly or indirectly through the k-wire 320 and/or the k-wire cap 300. In an example shown in FIGS. 22-25, the k-wire 320 may be inserted into and/or through the channel 212 of the sleeve 200 and may further be inserted into a second channel (not shown) extending through the punch 322 to contact and/or extend into a portion of the punch tip 326. As shown in FIG. 24, an exposed end 321 of the k-wire 320 is configured to receive the k-wire cap 300 and may extend outside of the punch 322 and/or the sleeve 200 in a direction opposite of that in which the sleeve 200 is inserted into the cut (i.e., in a direction from the second end 204 of the sleeve 200 toward the first end 202 of the sleeve 200).

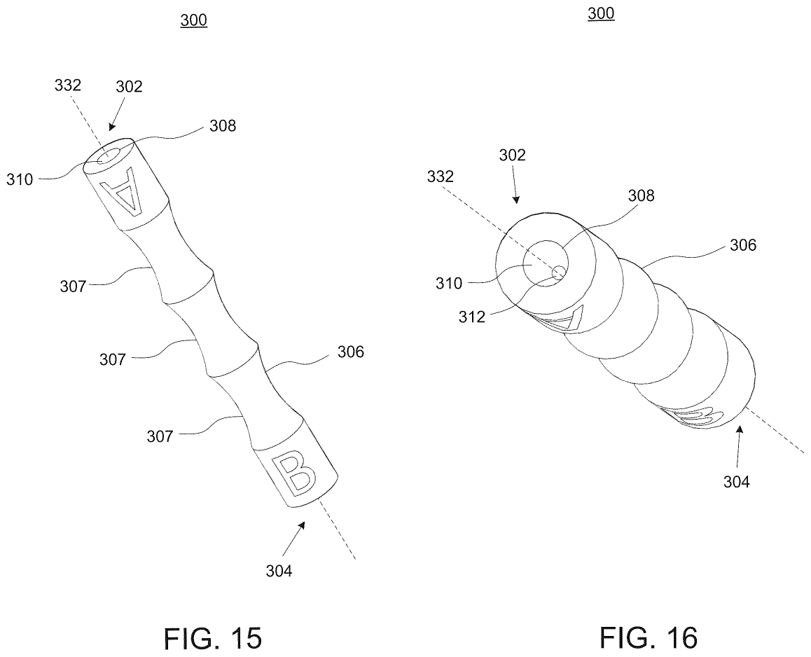

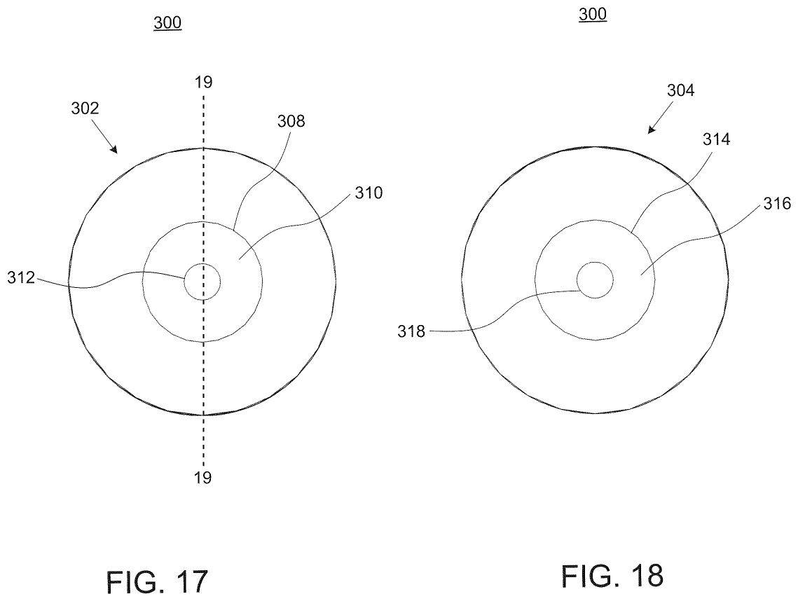

As shown in FIGS. 15-19, the k-wire cap 300 may include a first end 302 having a first opening 308 to a first cavity or long cavity 312, a second end 304 having a second opening 314 to a second cavity or short cavity 318, and a body 306 extending from the first end 302 to the second end 304. The body 306 of the k-wire cap 300 may include circumferential ribs or protrusions 307 to assist in gripping and/or ergonomically controlling the k-wire cap 300. The long cavity 312 may extend from the first opening 308 toward the second end 304. In addition, the long cavity 312 may extend from the first opening 308 toward the short cavity 318. The short cavity 318 may extend from the second opening 314 toward the first end 302. In addition, the short cavity 318 may extend from the second opening 314 toward the long cavity 312. In an embodiment shown in FIGS. 17-19, the long cavity 312 and the short cavity 318 may extend towards each other along a second longitudinal axis 332 extending through a center of the k-wire cap 300 from the first end 302 to the second end 304. In an example, the long cavity 312 may be centered within the first end 302 and the short cavity 318 may be centered within the second end 304. The first end 302 and the second end 304 of the k-wire cap 300 may be substantially flat and/or planar to provide a surface on which the first impact and the second impact may be applied (directly or indirectly) to the k-wire cap 300. When the k-wire cap 300 is impacted, the punch 322 and/or the k-wire 320 may be indirectly impacted, as described above.

The long cavity 312 and/or the short cavity 318 may be configured (e.g., sized, shaped and/or dimensioned) to receive at least a portion of the k-wire 320 including the exposed end 321. When at least a portion of the k-wire 320 is received in the channel 212 and the exposed end 321 is received in either the long cavity 312 or the short cavity 318, the k-wire 320 may align the k-wire cap 300 with the punch 322 and/or the sleeve 200. For example, the long cavity 312 may be configured to receive (e.g., slide over) the exposed end 321 such that the first end 302 of the k-wire cap 300 contacts the punch 322 when the k-wire 320 is received in the long cavity 312, as shown in FIG. 23. Similarly, the short cavity 318 may be configured to receive (i.e., slide over) the exposed end 321 such that the k-wire cap 300 is aligned with the punch 322 and/or the sleeve 200. In such an example, the k-wire 320 secures the k-wire cap 300 in place relative to the punch 322 such that the components are aligned, reducing the risk that the first impact may be applied incorrectly. The alignment indicator 224 may assist in determining whether the components are properly aligned with each other and/or with the soft tissue and/or bone of the patient.

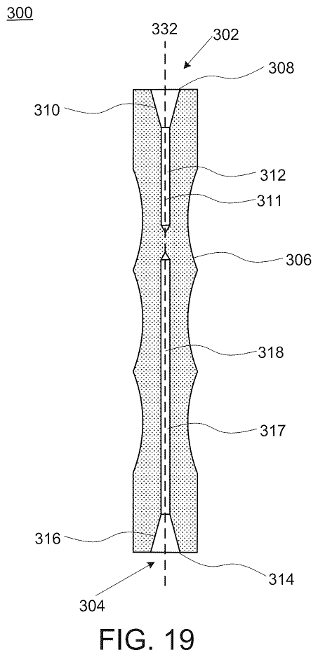

In an embodiment shown in FIG. 19, the long cavity 312 may include a first angled, tapered, or beveled surface or countersink 310 extending from the first opening 308 to a first bore-hole 311 such that the k-wire 320 may be guided by the first countersink 310 into the first bore-hole 311 when inserted into the long cavity 312. Similarly, the short cavity 318 may include a second angled, tapered, or beveled surface or countersink 316 extending from the second opening 314 toa second bore-hole 317 such that the k-wire 320 may be guided by the second countersink 316 when inserted into the short cavity 318. The first countersink 310 and the second countersink 316 may thereby make the insertion of the k-wire 320 (e.g., insertion of the exposed end 321) into the long cavity 312 and/or the short cavity 318 easier. The first countersink 310 may be, for example, configured (e.g., sized, shaped and/or dimensioned) to be the same as the second countersink 316.

In an embodiment, the long cavity 312 of the k-wire cap 300 may be configured to facilitate the first impact applied to the punch 322 and/or the k-wire 320. For example, when the k-wire 320 is received in the long cavity 312 such that the first end 302 of the k-wire cap 300 contacts the punch 322 as described above, the k-wire cap 300 works together with the first gap 328 to ensure the punch 322 and/or the k-wire 320 is driven into and/or through the tendon and/or the bone to the first predetermined depth, as shown in FIG. 23. The first impact may be applied to the second end 304 of the k-wire cap 300 such that the force of the first impact transfers through the k-wire cap 300 to the punch 322 and/or the k-wire 320, which forces the punch 322 further into the channel 212 of the sleeve 200 such that the first gap 328 is closed. The first impact may thus allow for the handle 324 of the punch 322 to meet and/or contact the first end 202 of the sleeve 200. When the first gap 328 is closed the punch tip 326 is driven or forced into the bone to the first predetermined depth. The handle 324 may prevent the first impact from driving the punch tip 326 beyond the first predetermined depth by acting as a stop preventing further movement of the punch 322 and/or the punch tip 326 in the same direction that the first impact is applied, for example, in the direction of the sleeve 200 being inserted into the cut.

In an embodiment, the short cavity 318 may be configured to facilitate the second impact applied to the k-wire 320. As shown in FIG. 24, when the k-wire 320 is received in the short cavity 318, the second end 304 of the k-wire cap 300 may face the punch 322 and a second gap 330 may exist between the k-wire cap 300 and the punch 322. The second gap 330 may be a distance which is equal to the second predetermined depth that is further than the first predetermined depth, for example, deeper. The second impact may be applied to the first end 302 of the k-wire cap 300 such that the force of the second impact transfers through the k-wire cap 300 to the k-wire 320, thereby driving or forcing the k-wire 320 into the bone to the second predetermined depth and closing the second gap 330 such that the second end 304 of the k-wire cap 300 contacts the punch 322. The punch 322 and/or the handle 324 may prevent the second impact from driving the k-wire 320 beyond the second predetermined depth by acting as a stop preventing further movement of the k-wire cap 300 beyond the point of contact between the k-wire cap 300 and the punch 322 in the same direction that the second impact is applied, for example, in the direction of the sleeve 200 being inserted into the cut.

To ensure that the k-wire 320 and/or the punch 322 (e.g., the punch tip 326) has/have been driven into the bone to the proper distance, for example, to the first predetermined depth and/or the second predetermined depth, in response to the first impact and/or the second impact, the bone 350 and/or the area surrounding the bone 350 which includes the tendon 352 may be imaged and/or scanned (e.g., CT scanned, x-rayed, etc.) and evaluated.

The method of the present disclosure enables reattachment of soft tissue to a pre-selected bony structure using an implant (e.g., the implant 100) which may include at least one (e.g., the portal hole 140). The method may utilize some or all the components of the system described above to perform a tenodesis procedure (e.g., to repair a tendon) and may reduce the number of steps required for surgical repair of a tendon whilst maintaining anatomic tensioning thereof. Although the method has been described with respect to the biceps tendon, it is understood and expressly contemplated that the implant 100 may be used with any tendon or like soft tissue structure. A tenodesis according to the method disclosed herein may therefore avoid the need to tenotomize a tendon and/or retrieve it, for example, using whip stitching outside the body. Maintaining anatomic tensioning during a tenodesis procedure according to the method disclosed herein may further eliminate the need for a surgeon to perform the challenging and non-reproducible step of precisely re-tensioning a tendon. The present method may therefore reduce and/or eliminate the risk of a tendon being secured improperly to a bone and may further reduce and/or eliminate the risk of various side effects or malformities which may result from a tenotomy and/or a tenodesis, such as pop-eye deformity.

The method will be described below with respect to a biceps tenodesis procedure, but it will be obvious, however, to those skilled in the art that aspects of the present disclosure may be practiced in other areas of the body, for example, to repair other tendons (e.g., torn and/or severed tendon(s)). The method may include multiple variations depending on the quality and/or hardness of the bone. The bone may be any bone suitable for soft tissue fixation. In an example shown in FIGS. 20-28, the method utilizes the punch 322, including the punch tip 326, to pierce a bone 350 to create a cavity in the bone 350 for securing the implant 100. The bone 350 may be, for example, a cortical or compact bone. In another example, the implant 100 shown in FIG. 29 which includes the hard tip 128 may be used to pierce the bone 350 instead of the punch 322 with punch tip 326 and/or the k-wire 320. In yet another example the punching and inserting of the k-wire 320 through the tendon 352 and the bone 350 may be unnecessary. In such an example, once the tendon 352 has been captured by the sleeve 200, the implant 100 may be inserted through the channel 212 of the sleeve 200 and impacted to secure the tendon 352 to the bone 350.

Turning to FIGS. 20-21, the method may include inserting the sleeve 200 into a wound, incision, or cut (not shown), the sleeve 200 having removably received the sleeve plug 214 within the channel 212 of the sleeve 200, such that the sleeve tip 220 and/or the sleeve plug 214 contacts the tendon 352 and/or the bone 350. The sleeve plug 214 may occupy the channel 212 during insertion into the cut to prevent soft tissue from getting trapped in the channel 212 and/or to prevent pressurized fluid from the cut to be expelled from the channel 212, among other things. The collar 206 may be gripped (e.g., by a surgeon) to assist in inserting, removing and/or otherwise ergonomically controlling the sleeve 200.

The sleeve plug 214 may then be used to locate the tendon 352, at which point the sleeve plug 214 is removed from the channel 212. Next, the sleeve tip 220 may capture the tendon 352 and hold the tendon 352 in place relative to the bone 350. Preferably, the sleeve 200 and/or the sleeve plug 214 locate and/or contact the tendon 352 and/or the bone 350 substantially at an expected and/or desirable implantation site for the implant 100. The contact point of the sleeve 200 and/or the sleeve plug 214 may be, for example, at the point along the bone 350 and/or the tendon 352 at which the tendon 352 is to be compressed to the bone 350 by the implant 100. The contact point may be selected such that the implant 100 may later be inserted through the channel 212 to pin the tendon 352 to the bone 350 without needing to reposition the sleeve 200. The sleeve plug 214 may then be removed from the channel 212 to expose the channel 212. The channel 212 may then be utilized for subsequent aspects of the surgical procedure.

In an embodiment, the sleeve tip 220 may hold the tendon 352 in place relative to the bone 350 via the first prong 226 and/or the second prong 228. Preferably, the sleeve tip 220 may contact the tendon 352 such that the natural anatomic tensioning of the tendon 352 is undisturbed (e.g., is maintained). The sleeve tip 220 may pin the tendon 352 to the bone 350 until the implant 100 is fully inserted as described in more detail below.

Once the sleeve plug 214 has been removed from the sleeve 200 to expose the channel 212, the punch 322 and/or the k-wire 320 may be inserted into the channel 212, as depicted in FIG. 22. The punch 322 may be configured (e.g., sized, shaped and/or dimensioned) such that the first portion 323 thereof fits into and/or slidingly engages (e.g., is capable of being inserted into) the channel 212. The first portion 323 preferably has a length which is longer than a length of the channel 212 such that the punch tip 326 extends beyond the sleeve tip 220 to contact the bone 350 and/or the tendon 352 leaving the first gap 328 between the punch handle 324 and the sleeve 200. As noted above, the first gap 328 may have a length which is equal to the first predetermined depth that the punch tip 326 is to be driven into the bone 350, which may allow the surgeon to visualize the punch tip 326 and k-wire 320 being driven into the bone. The k-wire 320 may then be inserted through the punch 322 which is within the channel 212 to contact and/or extend through the punch tip 326 to position an exposed end 321 of the k-wire 320 in a direction opposite of the inserted portion of the k-wire 320 in the punch 322 and/or the channel 212, for example, in a direction extending away from the cut. The k-wire 320 may extend into and/or through the punch tip 326. The surgeon may then use the punch handle 324 to ergonomically control the punch 322 throughout the procedure.

The k-wire cap 300 may be placed over the exposed end 321 of the k-wire 320 such that the exposed end 321 is received in the long cavity 312 and the k-wire cap 300 contacts the punch 322, as shown in FIG. 23. The long cavity 312 may have a length which is equal to or greater than a length of the exposed k-wire 320 from the exposed end 321 to the punch 322 and may therefore permit the first impact to be applied to the punch 322 and the k-wire 320 such that both are driven into the bone 350 to the same distance. The first impact may be made to prevent driving the k-wire 320 further into the bone 350 than the punch tip 326. As noted above, the punch tip 326 may be hollow and include a channel configured to permit the k-wire 320 to extend through and beyond the punch tip 326 and into the bone 350 to the second predetermined depth which may be further than the first predetermined depth. The k-wire cap 300 may therefore ensure that the k-wire 320 avoids being driven further than the punch tip 326 in a direction towards the tendon 352 and/or the bone 350 in response to the first impact.

The k-wire cap 300 may utilize the k-wire 320 for lateral stability. That is, the k-wire cap 300 may be placed over the k-wire 320 to align the k-wire cap 300 with the punch 322 and/or the sleeve 200. Whenever the k-wire cap 300 is placed over the k-wire 320, it may be that the k-wire cap 300, the k-wire 320, the punch 322, and/or the sleeve 200 are aligned along the second longitudinal axis 332. This alignment may reduce the risk of components slipping or moving out of place when the first impact is applied. The alignment of these components may also assist in directing the force of the first impact substantially in the same direction as the inserting of the punch 322 into the channel 212 of the sleeve 200. The alignment indicator 224 may assist in determining whether the components are properly aligned.

The first impact may then be applied, for example, by hammering or malleating, to the second end 304 of the k-wire cap 300. The second end 304 may be substantially flat or planar surface to allow for solid, controllable contact when applying the first impact. The k-wire cap 300 may impact the punch 322 and/or the k-wire 320 in response to the first impact. The first impact may then drive the punch tip 326 through the tendon 352 and into the bone 350 to the first predetermined depth, such that the first gap 328 between the punch handle 324 and the sleeve 200 may be closed and the punch handle 324 may contact the sleeve 200. The punch handle 324 may thus act as a stop which may prevent the punch tip 326 from being driven into the bone 350 beyond the first predetermined depth in response to the first impact. As noted above, the region including the bone 350 and the tendon 352 may be imaged or scanned here to determine whether the punch tip 326 has been properly driven into the bone 350 to the first predetermined depth.

Once the punch tip 326 has pierced the bone 350 to the first predefined depth, the k-wire cap 300 may be reversed and/or flipped over such that the exposed end 321 may be received within the short cavity 318 of the k-wire cap 300 and a second gap 330 exists between the k-wire cap 300 and the punch 322, as shown in FIG. 24. The second gap 330 may have a length which is equal to the second predetermined depth, as described above. The second predetermined depth may be the depth that the k-wire 320 is expected to be driven into the bone 350 in response to the second impact applied to the k-wire 320. In some embodiments, specifically with respect to biceps tenodesis procedures, the second predetermined depth may be about one inch. In other embodiments, the second predetermined depth may be some other depth depending on a variety of factors including surgeon preference and/or patient idiosyncrasies, among other factors.

The second impact may then be applied, for example, by hammering or malleating, to the first end 302 of the k-wire cap 300. The first end 302 may be substantially flat or planar surface to allow for solid, controllable contact when applying the second impact. The k-wire cap 300 may impact the k-wire 320 in response to the second impact to drive the k-wire 320 through the punch tip 326 and into the bone 350 to the second predetermined depth. The second impact may allow the second gap 330 between the k-wire cap 300 and the punch 322 to be closed and the k-wire cap 300 may contact the punch 322. The punch 322 may act as a stop which may prevent the k-wire 320 from being driven into the bone 350 beyond the second predetermined depth in response to the second impact. As noted above, the region including the bone 350 and the tendon 352 may be imaged or scanned to determine if the k-wire 320 has been properly driven into the bone 350 to the second predetermined depth.

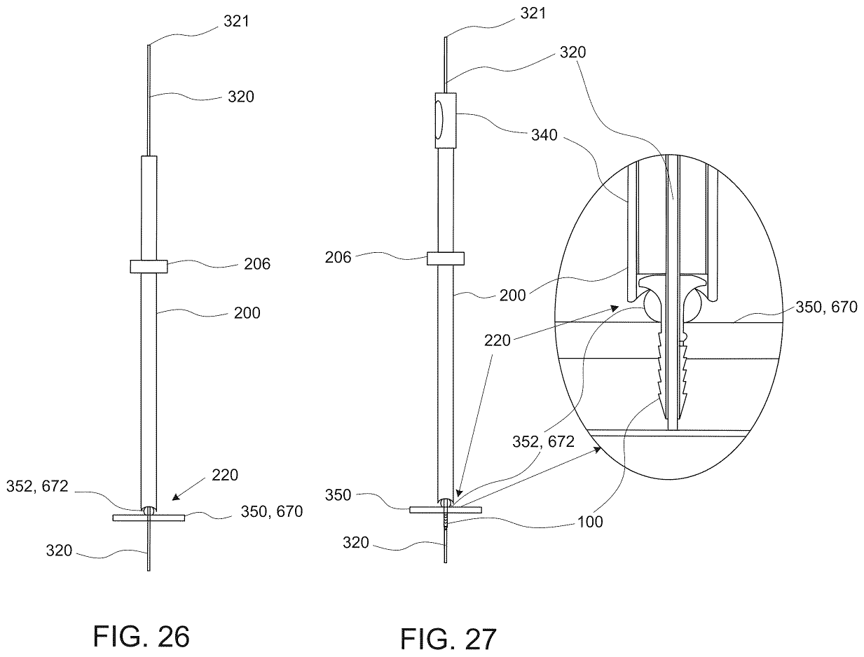

Once the k-wire 320 and the punch tip 326 have been inserted through the tendon 352 and into the bone 350, the k-wire cap 300 may be removed and the punch 322 may be back tapped (e.g., by hammering or malleating) and removed, leaving the sleeve 200 and the k-wire 320 in place, as shown in FIGS. 25-26. Removal of the punch 322, including the punch tip 326, may leave a cavity in the bone 350 having a depth which is equal to the first predetermined depth and is configured to receive the implant 100. At this point, the implant 100 may be prepared to be inserted. By creating the cavity in the bone 350 according to the method disclosed herein, the surgeon may avoid the need to drill the bone 350. The present method may enable a surgeon to repair the tendon 352 by compressing the tendon 352 wherever the surgeon prefers along the bone 350. For example, the present method may enable a surgeon to compress the tendon 352 anywhere along a humeral bone during a biceps tenotomy, for instance, anywhere from the epiphyseal line of the humerus to a mid-shaft of the humerus on the lateral to anterolateral side of the bone.

An inserter 340 may be used to assist in inserting the implant 100 in the cavity in the bone 350 left by the removal of the punch tip 326, as shown in FIG. 27. More specifically, the inserter 340 may assist in inserting the implant 100 over, around and/or down the exposed end 321 of the k-wire 320, the k-wire 320 being received in the cannula 130 of the implant. The inserter 340 may also assist in inserting the implant 100 into and/or through the channel 212 of the sleeve 200 to reach the cavity in the bone 350. The inserter 340 may further be configured (e.g., sized, shaped and/or dimensioned) to engage the counter-bore 136 during the insertion of the implant 100 into and/or through the channel 212. The counter-bore 136 may provide a flat surface on which to apply a third impact the implant 100 to drive the implant 100 into the bone 350, as described below. While in the embodiment discussed the inserter 340 is its own instrument, it is explicitly contemplated that the sleeve plug 214 and/or the inserter 340 may be configured (e.g., sized, shaped and/or dimensioned) to also perform the function of the other (e.g., the inserter 340 and/or the sleeve plug 214, respectively), and thus in some embodiments there may be only one instrument performing the function of both the sleeve plug 214 and the inserter 340.

The channel 212 of the sleeve 200 and/or the k-wire 320 may assist in guiding the implant 100 toward and/or into the cavity in the bone 350 during insertion of the implant 100.

For example, the punch tip 326 may be configured (e.g., sized, shaped and/or dimensioned) to direct the k-wire 320 through the punch tip 326 and through a center of the cavity in the bone 350 left by the removal of the punch tip 326 in response to the second impact. The k-wire 320 may then be used to guide and/or center the implant 100 with respect to the cavity in the bone 350. During the insertion of the implant 100 into the channel 212, the implant 100, the k-wire 320, the sleeve 200, and/or the inserter 340 may be longitudinally aligned along the first longitudinal axis 106 of the implant 100.

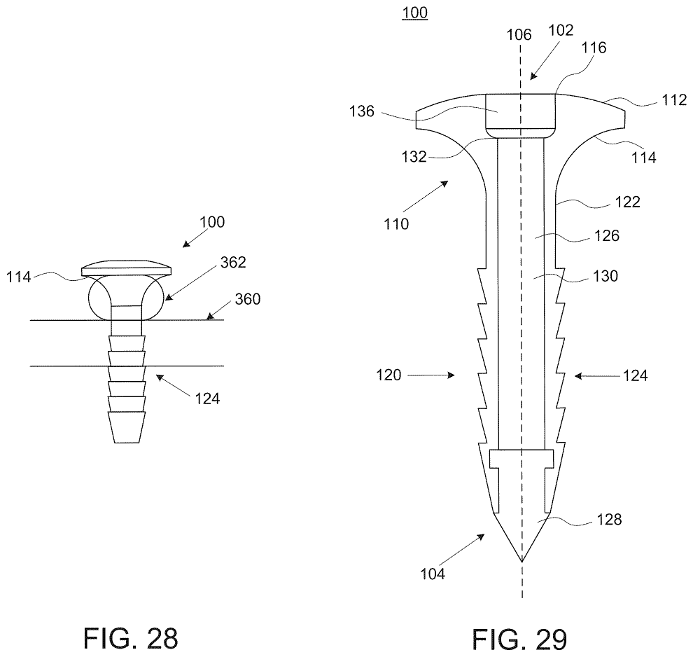

The implant 100 may slide or otherwise progress down the k-wire 320 to contact the tendon 352, at which point the third impact may be applied, for example, by hammering or malleating, to the inserter 340 to drive the implant 100 through the tendon 352 and/or the bone 350 to the first predetermined depth to fully seat the implant 100. As shown in FIG. 28, the tendon 352 may be compressed to the bone 350 by the bottom surface 114 of the implant 100 while maintaining the natural anatomic tensioning of the tendon 352. When inserted, the ridges 124 of the implant 100 may engage the bone 350 to secure the implant 100 in place relative to the bone 350. Once the implant 100 has been properly secured within the cavity in the bone 350, then the sleeve 200, inserter 340, and k-wire 320 may be removed to complete the surgical procedure. In some embodiments, completing the surgical procedure may also include stitching or otherwise closing the cut or incision.

When the implant 100 is inserted, the cannula 130 of the implant 100 may contact an intramedullary region of the bone 350 and may be in fluid communication with the intramedullary region of the bone 350. Also, when the implant 100 is inserted, the portal hole 140 may further be in fluid communication with the region between the bone 350 and the tendon 352. The implant 100, via the cannula 130 and the portal hole 140, may therefore facilitate the transport of biological agents, for example, healing agents, from the intramedullary region to the region between the bone 350 and the tendon 352 which may promote healing of the bone 350, the tendon 352, and/or surrounding tissue.

As noted above, in some embodiments, depending on factors including the hardness of the bone 350 and patient idiosyncrasies, the additional steps of punching and/or inserting the k-wire 320 through the tendon 352 and/or the bone 350 may be unnecessary. In such circumstances, once the tendon 352 has been captured by the sleeve tip 220 of the sleeve 200, the implant 100 is placed down the channel 212 of the sleeve 200 and impacted to drive the implant 100 into the bone 350 and secure the tendon 352 to the bone 350. The implant 100 may be placed down the channel 212 with, for example, the inserter 340. The impact may be applied indirectly to the implant 100 by impacting, (e.g., by hammering or malleating) the inserter 340 or another instrument which is engaged with the implant 100. In some embodiments, driving the implant 100 into the bone 350 may include the hard tip 128.

Also as noted above, in some embodiments, depending again on factors including the hardness of the bone 350 and patient idiosyncrasies, the implant 100 with the hard tip 128, shown in FIG. 29, may be used to pierce the bone 350 instead of the punch 322 with the punch tip 326 and/or the k-wire 320. Where the bone 350 is hard, the punch tip 326 and/or the k-wire 320 may be insufficient to create the cavity in the bone 350 to receive the implant 100, and may buckle, snap, or otherwise break if inserted with force and met with resistance from the hard bone 350. In such circumstances the steps involving the punch 322 and the k-wire 320 may be impractical, but the implant 100 may also be at risk of buckling, snapping, or otherwise breaking. The hard tip 128, however, may be stronger, for example, by being formed of a harder material such that the implant 100 is more resistant to breaking than the punch tip 326 and/or the k-wire 320, and therefore the implant 100 may be impacted to create the cavity in the bone 350.

Referring now to FIGS. 11-26 and 30-52, another embodiment of the system for soft tissue fixation will now be described in detail. Specifically, an embodiment of the system that includes an anchor or implant 400 and an inserter 640 will now be described. In the alternative embodiment of the system, the implant 400 may be, for example, the same, similar to, and/or include overlapping features with implant 100, and the inserter 640 may be, for example, the same, similar to, and/or include overlapping features with the inserter 340. This embodiment of the system for soft tissue fixation (e.g., compressing or squishing a soft tissue to a pre-selected bone/bony structure (e.g., a cortical bone)) may include the tack or implant 400, the sleeve 200, the sleeve plug 214, and/or the inserter 640. In further embodiments, the second system may include the fixator or k-wire 320, the fixator cap or k-wire cap 300, and/or the punch 322.

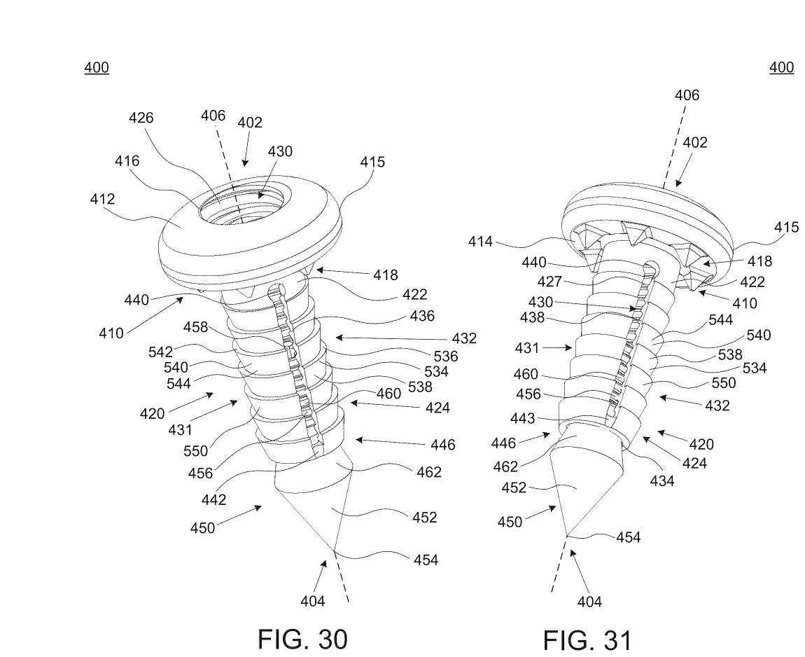

As shown in FIGS. 30-37, the implant 400 for soft tissue fixation may have a first end 402 and a second end 404. The implant 400 may further include a head 410 connected to a stem 420. The stem 420 may include a hard tip 450 connected to the stem 420 on an end opposite the head 410. The head 410, the stem 420, and the hard tip 450 may extend from the first end 402 to the second end 404. The head 410 may extend from the first end 402 to the stem 420. The stem 420 may extend away from a bottom surface 414 of the head 410 towards the second end 404 of the implant 400. In some embodiments, the hard tip 450 may extend from the stem 420 to the second end 404.

The implant 400 may be, for example, a singular, integral, or monolithic piece (i.e., of one-piece construction), or may be formed from a plurality of components that are coupled (e.g., rigidly coupled) together to form the implant 400. For example, in some embodiments, the head 410, the stem 420, and/or the hard tip 450 may be monolithically formed, whereas in other embodiments the head 410, the stem 420 and/or the hard tip 450 may be modular to allow various differently shaped components to be combined, as needed. The head 410, the stem 420 and/or the hard tip 450 may be provided to fully secure a soft tissue (e.g., a tendon) in and/or to a bone. The implant 400 may be made by machine, for example, by machining or injection molding, or by 3D printing, among other processes. In an example, the implant 400 may be made out of PEEK, stainless metal, ultra-high molecular weight polyethylene, or a resorbable material, or a combination of any of the foregoing. For example, in some embodiments, the hard tip 450 may be made of PEEK, and the head 410 and the stem 420 may be made of some other material. In further embodiments, the head 410 may be made of a different material than the stem 420.

With continued reference to FIGS. 30-37, the head 410 may be substantially circular and may further include a top surface 412 opposite to the bottom surface 414. In some embodiments, the head 410 may be a different shape, such as an ovular shape. In further embodiments, the head 410 may be a non-rounded shape, such as a polygon. The top surface 412 may be connected to the bottom surface 414 by the side surface 415. The head 410 may also include a first opening 416 of a through-bore or cannula 430. The first opening 416 may be located in or on the head 410, for example, in or on the top surface 412. The top surface 412 may be, for example, curved, angled, or flat as the top surface 412 extends between the first opening 416 and the side surface 415. The bottom surface 414 may be configured (e.g., sized, shaped and/or dimensioned) to compress a soft tissue (e.g., a tendon) to the bone when the implant 400 is inserted into a bone, such that the tendon may be fixed in place relative to the bone, as described in more detail below. For example, the bottom surface 414 may be flat or planar as it extends between the stem 420 and the side surface 415.

In some embodiments, the bottom surface 414 of the head 410 may also include a gripping means, for example, a plurality of spikes or protrusions 418. The protrusions 418 may be configured (e.g., sized, shaped and/or dimensioned) to engage with a soft tissue (e.g., a tendon) during insertion of the implant 400 into a cut or incision. For example, the protrusions 418 may be configured (e.g., sized, shaped and/or dimensioned) to provide an initial grip or otherwise engage with a tendon to assist with pinning the tendon to a desired location on a bone. The protrusions 418 may also prevent the implant 400 from rotating once inserted into a cut or incision by grasping soft tissue (e.g., a tendon) or bone to anchor the implant 400 in place relative to the soft tissue or bone. In the embodiment shown, there are six of the protrusions 418, but it is contemplated that alternative embodiments may feature more or less than six protrusions. The protrusions 418 may also be configured (e.g., sized shaped and/or dimensioned) to engage with a bone, for example, by contacting and/or digging into the bone, which may further secure a tendon thereto.

As shown in FIG. 33, the protrusions 418 may be slightly inset from the side surface 415 towards the stem 420. Specifically, the system and method (described in more detail below) may include a grommet or surgical dam (not shown) through which a bone and/or a tendon is accessed during surgery, and through which the implant 400 is inserted to reach the bone and/or the tendon or other soft tissue. During the insertion of the implant 400, there is a risk that the implant 400 (e.g., the protrusions 418) will catch along an interior surface of the surgical dam, which may obstruct the user's (e.g., surgeon's) ability to place the implant 400 at the necessary or desired location. The insetting of the protrusions 418 may minimize the risk of the implant 400 catching or otherwise gripping an interior surface of the surgical dam, therefore, improving the insertion of the implant 400 into and/or through a surgical cut and/or surgical dam.

In some embodiments, the implant 400 may not include a head 410 and there may instead be a lip or other extension (not shown) from the stem 420 (e.g., at an end of the stem 420 which is opposite the hard tip 450) which is configured (e.g., sized, shaped and/or dimensioned) to pin a tendon to a bone. The lip or other extension may therefore include one or all of the protrusions 418 to assist in pinning the tendon to the bone. In addition, the lip or other extension may, for example, have a width and/or thickness in a direction between the first end 402 and second end 404 of the implant 400 that is smaller than the width and/or thickness of the head 410 extending in the same direction.

As shown in FIGS. 30-31 and 33-37, the stem 420 may include an exterior or outer surface 422 and at least a portion of the cannula 430 may extend through at least a portion of the stem 420. The cannula 430 may include the first opening 416, a first inner surface 426, a second inner surface 427, and/or a second opening 434. In some embodiments, one or both of the first inner surface 426 and/or the second inner surface 427 may include threading which may be configured (e.g., sized, shaped and/or dimensioned) to engage with a tool, such as the inserter 640 and/or the hard tip 450, as described in more detail below. The threading of the first inner surface 426 may be, for example, the same or different from the threading of the second inner surface 427. The stem 420 may also include a plurality of teeth or ridges 424 positioned along the outer surface 422 of the stem 420. The plurality of ridges 424 may extend away from the stem 420 to assist with anchoring the implant 400 into the patient.

The stem 420 may further include a first side slot or slit 436 and/or a second side slot or slit 438. The first side slit 436 and/or the second side slit 438 may extend, for example, longitudinally along at least a portion of the stem 420 and/or from the cannula 430 to the outer surface 422 of the stem 420, as shown in FIGS. 30-31 and 35. For example, the side slit(s) 436, 438 may extend from a position below or distal to the head 410 to a position above or proximal to the hard tip 450 along the length of the stem 420. In some embodiments (not shown), there may be additional slits (e.g., additional structures which are identical or substantially similar to the first side slit 436 or the second side slit 438) positioned along the length of the stem 420 and/or optionally circumferentially positioned around the stem 420. The first side slit 436 and the second side slit 438 may collectively divide at least a portion of the stem 420 into a first expandable leg or wing 431 and a second expendable leg or wing 432. The first side slit 436 and the second side slit 438 may thus separate the stem 420 and may further allow for the stem 420 to be separated and/or expanded to transition the implant 400 from a resting, closed and/or first position or state 446 (FIGS. 30-37) to an active, open, expanded and/or second position or state 448 (FIGS. 44-46) to anchor the implant 400 in place in or relative to a bone and/or relative to a tendon, as described in more detail below.

As shown in FIGS. 30-31, 35-37, and 44, the first side slit 436 may include a first venting hole 440 and the second side slit 438 may include a second venting hole 441. The first venting hole 440 and/or the second venting hole 441 may allow for and/or permit the passage of biological agents (e.g., bone marrow and/or healing agents) between the cannula 430 of the implant 400 and the bone into which the implant 400 is inserted. For example, when the implant 400 is inserted, the stem 420 of the implant 400 may be positioned at least partially within the intramedullary canal of the bone, such that biological agents (e.g., healing agents) may pass through second opening 434 and through the cannula 430 of the stem 420 to reach the first venting hole 440 and/or the second venting hole 441. The first venting hole 440 and/or the second venting hole 441 may therefore be positioned and/or configured (e.g., sized, shaped and/or dimensioned) such that, when the implant 400 is inserted into the bone to pin the tendon thereto, the first venting hole 440 and/or the second venting hole 441 are in fluid communication with a space or area between the tendon and the bone. Such fluid communication may permit transportation or delivery of a patient's natural biological agents (e.g., healing agents) from within the intramedullary canal to enter the cannula 430 and then travel through the cannula 430 and through the first venting hole 440 and/or the second venting hole 441 to access the space and/or area between the compressed tendon and the bone, promoting healing of surgical tissues (e.g., the bone, the tendon, and/or surrounding tissue(s)).

To facilitate the separation and/or expansion of the first wing 431 and the second wing 432, the first side slit 436 may further include a first breakaway point 442 and the second side slit 438 may further include a second breakaway point 443. In some embodiments, the first breakaway point 442 and the second breakaway point 443 may be disposed towards the second end 404. For example, the first breakaway point 442 and the second breakaway point 443 may form part of the second opening 434, as shown in FIGS. 30-31 and 36. The first breakaway point 442 and the second breakaway point 443 may be configured (e.g., sized, shaped and/or dimensioned) to break, bend, deform and/or snap to disconnect the first wing 431 from the second wing 432 about the second opening 434, permitting the wings 431, 432 to expand away from each other to widen the stem 420, which may lock the implant 400 in place in a bone. The breaking of the first breakaway point 442 and the second breakaway point 443 may product a sound (e.g., a pop or snap) which may indicate to a user that one or both of the breakaway points 442, 443 have broken as intended and therefore the implant 400 has at least begun the transition from the first state 446 to the second state 448.

The first breakaway point 442 and the second breakaway point 443 may be formed of the same material as the stem 420. The thickness (e.g., wall thickness) of the breakaway points 442, 443 and the remainder of the stem 420 may, for example, extend between an exterior surface of the stem 420 and the cannula 430. In some embodiments, the breakaway points 442, 443 may be thinner (e.g., have a thinner wall thickness) than the remainder of the stem 420 to ensure that it is the breakaway points 442, 443, as opposed to some other component of the implant 400, which break during the method as described below. Alternatively, the breakaway points 442, 443 may be thinner than at least an immediate surrounding area of the remainder of the stem 420. In some embodiments, the first breakaway point 442 and/or the second breakaway point 443 may be about half the thickness and/or strength of the remainder of the stem 420. Alternatively, the breakaway points 442, 443 may be formed by scoring, notching, or any other method which ensures the breaking of the breakaway points 442, 443. In alternative embodiments, the breakaway points 442, 443 may be formed of a different, weaker material than the remainder of the stem 420 to facilitate the desired breaking or snapping thereof.

To ensure that components other than the breakaway points 442, 443 do not break during the transition of the implant 400 from the first state 446 to the second state 448, the first venting hole 440 and/or the second venting hole 441 may be configured (e.g., sized, shaped and/or dimensioned) to act as stress reducers. For example, the venting holes 440, 441 may have a decreased amount of material as compared to a remainder of the stem 420, which may facilitate the bending or deformation of the stem 420 at the venting holes 440, 441 during the expansion of the wings 431, 432 (e.g., during the transition from the first state 446 to the second state 448). Also, in some embodiments, the venting holes 440, 441 may have a rounded shape such that the venting holes 440, 441 act as stress reducing radii limiting the expansion of the implant 400 to the expansion of the wings 431, 432 and minimizing and/or preventing the integrity of the head 410 from being compromised during the transition from the first state 446 to the second state 448. In other embodiments, there may be no venting holes 440, 441 and there may only be stress reducers.

With continued reference to FIGS. 30-31 and 34-37, the ridges 424 may be disposed along a portion of the outer surface 426 of the stem 420. In other embodiments (not shown), the ridges 424 may extend along all of the outer surface 426 of the stem. In some embodiments, the ridges 424 may be disposed towards the second end 404 of the implant 400, such that a portion of the stem 420 which connects to the head 410 does not include the ridges 424. The placement of the ridges 424 toward the second end 404 of the implant 400 may allow for the ridges 424 to best engage the bone to assist in fully securing the implant 400 thereto and/or therein. In such an embodiment, the portion of the stem 420 which connects to the head 410 may be, for example, circumferentially smooth.

The ridges 424 may be substantially circumferentially continuous along the outer surface 426. In some embodiments, some or all of the ridges 424 may be split or broken apart, for example, by the first side slit 436 and/or the second side slit 438, such that a portion of each of the ridges 424 may be located on the first wing 431 and another portion of each of the ridges 424 may be on the second wing 432. In other embodiments, the ridges 424 may also be, for example, split or broken as the ridges 424 extend between the first side slit 436 and the second side slit 438 on each wing 431, 432 forming portions of ridges 424 along the stem 420.

A first ridge 534 of the ridges 424 may have a first upper surface 536 which may be substantially flat or planar and which may substantially face the first end 402, and a first lower surface 538 which may be inclined, tapered or angled towards the second end 404, as shown in FIGS. 30-31 and 34-35. The first lower surface 538 may be inclined, tapered, or angled, for example, such that the thickness of the first ridge 534 decreases in a direction from the first end 402 to the second end 404. The first upper surface 536 may contact or be coupled to a second ridge 540 of the ridges 424, and the first lower surface 538 may contact or be coupled to a third ridge 550 of the ridges 424. In some embodiments, each of the ridges 424 may be longitudinally aligned along a first longitudinal axis 406 of the implant 400.

The ridges 424 (e.g., the upper surface and/or the tapering of the incline described above) may be configured (e.g., sized, shaped and/or dimensioned), for example, to act as barbs or fasteners to engage the bone when the implant 400 is inserted into the bone to hold the implant 400 in place relative to the bone and/or to prevent pullout of the implant 400. As the surgical site heals, the bone may heal around any or all of the ridges 424 to further secure the implant 400 (and thus, the tendon) in place relative to the bone.

In alternative embodiments, the ridges 424 may be of any size or shape which resists and/or assists in preventing pullout of the implant 400 from the bone following insertion. For example, while the upper surfaces 536, 542 of the ridges 534, 540 are shown as extending from the outer surface 422 of the stem 420 the same distance and having the same angle, it is also contemplated that the upper surfaces 536, 542 may extend away from the outer surface 422 of the stem 420 different distances and/or at different angles. In an embodiment, the upper surfaces 536, 542 may have different widths or circumferences and corresponding lower surfaces 538, 544 that taper along the length of the stem 420, with the larger width or circumference preferably being closest to the head 410 or stem 402 of the implant 400.

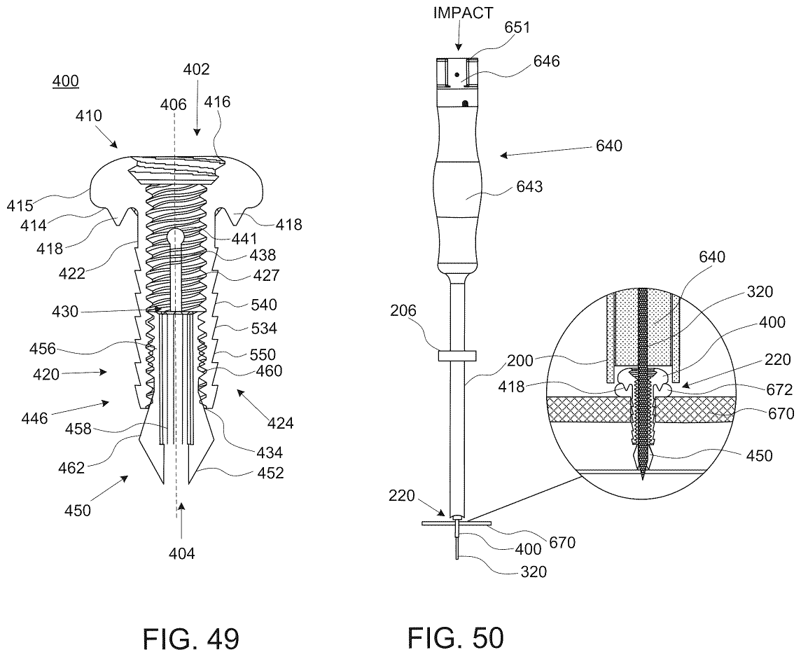

At least a portion of the cannula 430 may be bound by the first inner surface 426 and/or the second inner surface 427, as shown in FIGS. 30-32 and 36-37. The cannula 430 may extend from the first opening 416 through the head 410 and through at least a portion of the stem 420 to the second opening 434. In some embodiments, the cannula 430 may extend completely through the head 410 and the stem 420, for example, along the first longitudinal axis 406 of the implant. In other embodiments, the second opening 434 may be blocked, plugged, or otherwise closed by the hard tip 450 when the implant 400 is in the first state 446. In some embodiments, the cannula 430 may be a partial cannula (not shown) and may extend through only a portion of the stem 420. In even further embodiments, the cannula 430 may extend completely through the head 410, the stem 420, and the hard tip 450, as shown in FIG. 49.

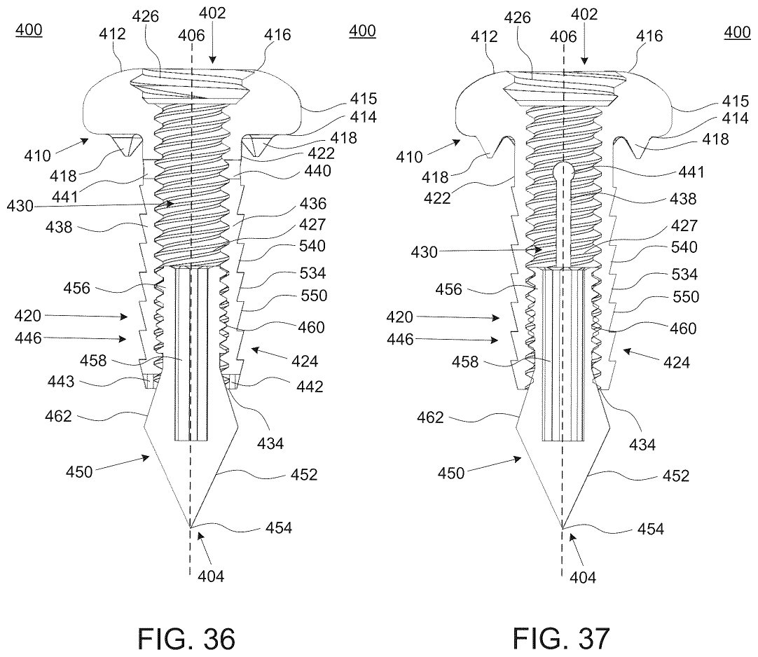

As shown in FIGS. 36-37, the first inner surface 426 may extend from the first opening 416 to the second inner surface 427, and the second inner surface 427 may extend from the first inner surface 426 to the second opening 434. The first inner surface 426 and the second inner surface 427 may be threaded, for example, to permit engagement thereof with various tools, such as the inserter 640 and/or the hard tip 450, as described in more detail below. The first opening 416, the cannula 430 (e.g., the first inner surface 426 and the second inner surface 427), and the second opening 434 may be in fluid communication with each other and/or with the first side slit 436 and/or the second side slit 438 (including the first venting hole 440 and/or the second venting hole 441, respectively), and/or with an exterior of the implant 400 to permit various instruments to be placed into the cannula 430 and to permit the passage or flow of biological agents through the cannula 430 and/or the first venting hole 440 and/or the second venting hole 441.

When the implant 400 is inserted into the bone, the second opening 434 and/or the cannula 430 may contact and/or be in fluid communication with an intramedullary canal of a patient. In some embodiments, the second opening 434 may only be in fluid communication with the intramedullary region of the bone following movement of the wings 431, 432 outwardly away from the hard tip 450 during a transition of the implant 400 from the first state 446 to the second state 448 as described in more detail below. In other embodiments, the cannula 430 may extend through the hard tip 450 to permit fluid communication between the cannula 430 and the intramedullary region both while the implant 400 is in the first state 446 and the second state 448. The first opening 416 and/or the first inner surface 426 may have a diameter which is larger than a second diameter of the second opening 434 and/or the second inner surface 427.

The hard tip 450 of the implant 400 may include a second head 452 connected to a second stem 456. The second head 452 may include a tip or point 454, for example, at an end of the second head 452 opposite of the connection to the second stem 456. When the implant 400 is in the first state 446, the second stem 456 of the hard tip 450 may be located in the cannula 430 (e.g., in the second opening 434) such that the second head 452 extends out of the second opening 434 to an exterior of the implant 400 and the point 454 faces away from the cannula 430. The point 454 may be sharp and may further be configured (e.g., sized, shaped and/or dimensioned) to be driven through soft tissue (e.g., a tendon) and bone to seat the implant 400. In alternative embodiments where the cannula 430 extends through the hard tip 450, there may be no point 454 and instead there may be another opening (FIG. 49). In such an embodiment, an opening in the bone into which the implant 400 is to be inserted may be formed through the use of the k-wire 320, the k-wire cap 300, and/or the punch 322, as described in more detail below.

The second stem 456 of the hard tip 450 may be configured (e.g., sized, shaped and/or dimensioned) to be received in the stem 420, for example, within the cannula 430, as shown in FIGS. 30-31 and 35-37. The second stem 456 may include a threading 460. The threading 460 may be configured (e.g., sized, shaped and/or dimensioned) to engage with a corresponding threading of the second inner surface 427 to allow the hard tip 450 to be drawn or otherwise pulled up through the cannula 430 towards the head 410 of the implant 400 in response to a twisting, turning or rotating of the hard tip 450, as described in more detail below with respect to the method of the present disclosure.

The hard tip 450 may be pre-assembled or pre-molded into the implant 400. For example, the implant 400 (e.g., the stem 420 and/or the cannula 430) may be formed around the hard tip 450 (e.g., around the second stem 456) or the hard tip 450 may be inserted into the implant 400 during formation thereof. Alternatively, the hard tip 450 may be screwed into the remainder of the implant 400 (e.g., the head 410 and the stem 420), for example, by inserting the second stem 456 into the cannula 430 (e.g., through the second opening 434) such that the threading 460 engages with the threading of the second inner surface 427. The hard tip 450 may then be rotated to advance the hard tip 450 into the cannula 430 along the second inner surface 427 in a direction from the second end 404 to the first end 402 to transition the implant 400 from the first state 446 to the second state 448 as described in more detail below.

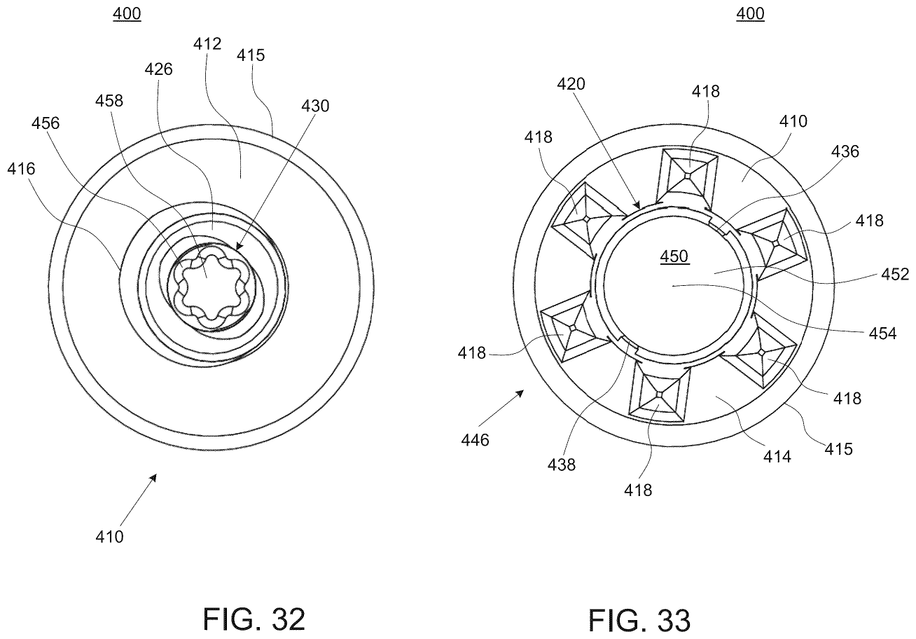

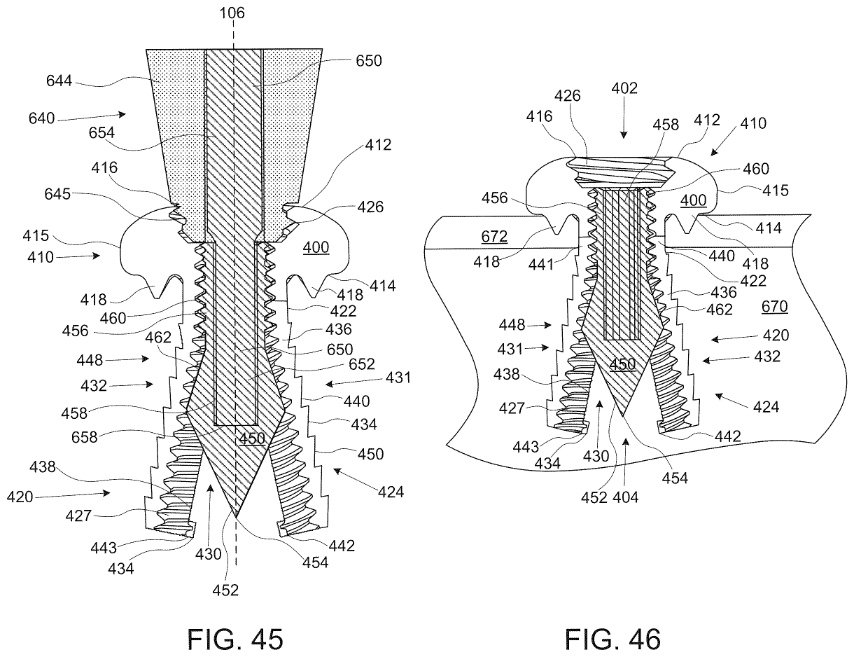

The hard tip 450 may further include a cavity 458, as shown in FIGS. 32 and 36-37. The cavity 458 may be configured (e.g., sized, shaped and/or dimensioned) to engage with a portion of the inserter 640 in order to transition the implant 400 from the first state 446 to the second state 448, as shown in FIG. 45. For example, a turning of the inserter 640 while engaged with the cavity 458 may result in the hard tip 450 progressing along the threading of the second inner surface 427 in a direction from the second opening 434 towards the head 410. As the hard tip 450 moves increasingly into the cannula 430, a pressure or force is applied to the second opening 434 (e.g., to the breakaway points 442, 443) by a tapered portion 462 of the hard tip 450 which has a diameter that is larger than a diameter of the second opening 434. The force applied to the second opening 434 by the tapered portion 462 as the hard tip 450 is drawn into the cannula 430 may thus be sufficient to cause the breakaway points 442, 443 to snap, break, or otherwise disconnect the first wing 431 from the second wing 432. Following such a break, the continued movement of the hard tip 450 through the cannula 430 towards the head 410 may cause the first wing 431 and the second wing 432 to expand or otherwise move away or diverge from each other in substantially opposite directions, which may widen at least a portion of the stem 420 to secure the implant 400 in place in or relative to a bone.

This movement of the wings 431, 432 away from each other is also referred to herein as the transitioning of the implant 400 from the first state 446 to the second state 448. Specifically, the implant 400 is in the first state 446 while the breakaway points 442, 443 are intact (e.g., before the breakaway points 442, 443 are broken or snapped by the hard tip 450 being drawn up into the cannula 430). The implant 400 is then in its second state following the breaking or snapping of the breakaway points 442, 443 and/or the expansion of the wings 431, 432. After the implant 400 has been inserted into a cut or incision (e.g., through a tendon and into a bone), the expansion of the wings 431, 432 away from each other as just described may seat the implant 400 in a bone (e.g., may hold the implant 400 in place relative to a patient's tendon and/or bone).

Turning now to FIGS. 11-26 and 38-52, a system is shown including the implant 400, the inserter 640, the sleeve 200, and/or the sleeve plug 214. In addition, the system may include the fixator or k-wire 320, the fixator cap or k-wire cap 300, and/or the punch 322.

The inserter 640 may extend from a first end 641 to a second end 642, and may in some embodiments include a handle 643, a shank or member 644, a knob 646 and a rod 650 having a tip 652, as shown in FIGS. 38-43 and 51-52. In some embodiments, the knob 646 may contact the handle 643 and/or may extend from or substantially from the first end 641 to the handle 643. The knob 646 may also include one or a plurality of knob protrusions 648, which may be ergonomically configured (e.g., sized, shaped and/or dimensioned) to be grasped by a user (e.g., a surgeon) to facilitate twisting of the knob 646. Further, the knob 646 may have an end face 651 located, for example, at the first end 641 of the inserter 640. The end face 651 of the knob 646 may be, for example, flat or substantially flat, or otherwise configured (e.g., sized, shaped and/or dimensioned) to receive the first impact to drive the implant 400 into the bone, as described in more detail below.

With continued reference to FIGS. 38-43 and 51-52, the handle 643 may extend substantially from the knob 646 to the member 644. The handle 643 may be ergonomically configured (e.g., sized, shaped and/or dimensioned) to be grasped and controlled by a user, for example, during an insertion of the implant 400 into a cut or incision (and/or into a grommet or surgical dam (not shown)), during a twisting of the knob 646, during a removal of the inserter 640 from the cut or incision (and/or from a grommet or surgical dam), during an expansion of the first wing 431 and the second wing 432 (e.g., as the implant 400 is transitioned from the first state 446 to the second state 448, as described in more detail below), and/or for any other purpose for which the inserter 640 may be used.

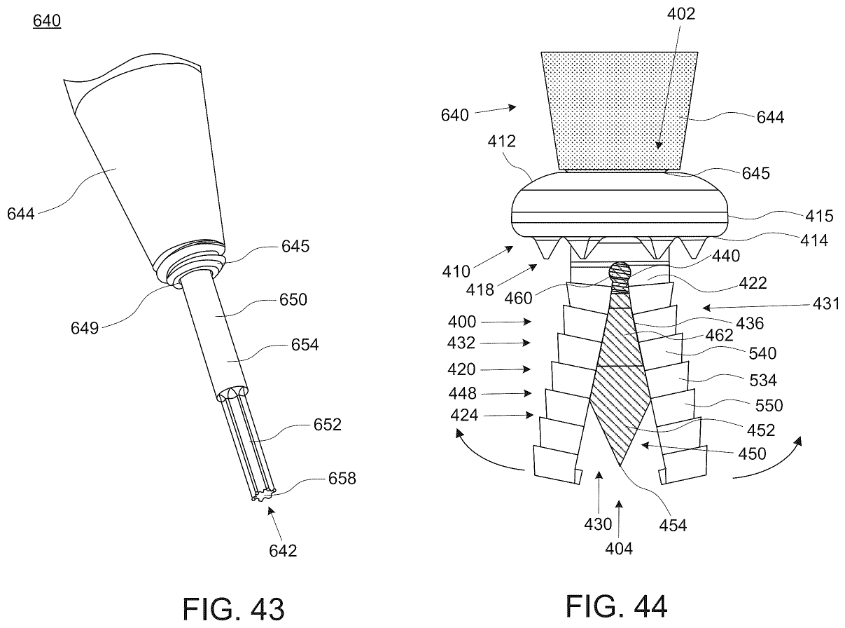

The member 644 may extend substantially from the handle 643 to an exposed portion of the rod 650 (e.g., to the tip 652 of the rod 650). The member 644 may also include a threaded end 645, for example, on an end of the member 644 opposite the connection of the member 644 to the handle 643. As shown in FIG. 43, the threaded end 645 may include an opening 649 through which the rod 650 may extend. Said another way, the rod 650 may protrude from the opening 649 of the inserter 640. The threaded end 645 may further be configured (e.g., sized, shaped and/or dimensioned) to engage with the implant 400, for example, with the threading of the first inner surface 426, to secure the inserter 640 to the implant 400 as described in more detail below with respect to the method of the present disclosure. The member 644 may be, for example, tapered from its'greatest diameter to the diameter of the threaded end 645. The diameter at the threaded end 645 being smaller than the greatest diameter of the member 644. The tapered portion may be, for example, positioned near the second end 642 of the inserter 640.

The rod 650 may include a first portion 654 and a tip 652, as shown in FIGS. 42-43. In some embodiments, the rod 650 may extend through an interior of the inserter 640, for example, longitudinally through at least a portion of the knob 646, the handle 643, and/or the member 644, as shown in FIG. 42. Said another way, the rod 650 may extend longitudinally through a center of the inserter 640 along a third longitudinal axis 656. The rod 650 may further extend and/or protrude through the opening 649 of the member 644 in a direction from the first end 641 towards the second end 642, such that the tip 652 of the rod 650 may exit the opening 649 and is exposed to an exterior of the inserter 640 for engagement with at least a portion of the implant 400. In some embodiments, the rod 650 may also extend and/or protrude out of an opposite end of the member 644 in a direction from the second end 642 towards the first end 641 to contact the knob 646.

As shown in FIGS. 42 and 52, a bar 647 may extend through an interior of the knob 646 in a direction substantially perpendicular to the third longitudinal axis 656 (e.g., substantially perpendicular to the direction of extension of the rod 650). The bar 647 may further spin, twist, turn and/or rotate in response to a corresponding spinning, twisting, turning and/or rotating of the knob 646 as described above. In some embodiments, the rod 650 may be coupled to the bar 647 such that a twisting, spinning, turning, and/or rotating of the knob 646 (and thus also of the bar 647) results in a corresponding twisting, spinning, turning and/or rotating of the rod 650. The knob 646 may therefore permit the rod 650, including the tip 652, to be rotated independently of the handle 643 and/or member 644, for example, about the third longitudinal axis 656. Thus, a user (e.g., surgeon) may hold the handle 643 of the inserter 640 steady while turning the knob 646 to rotate the rod 650.

The tip 652 of the rod 650 may be configured (e.g., sized, shaped and/or dimensioned) to engage with the cavity 458 of the second stem 456 of the implant 400. For example, the tip 652 may be configured (e.g., sized, shaped and/or dimensioned) to be inserted into the cavity 458, as shown in FIG. 45. In some embodiments, the tip 652 may have a polygonal end 658 of any shape and/or dimension which permits engagement thereof with a corresponding geometry of the cavity 458. In an example, the tip 652 and the polygonal end 658 may have the shape of a standard screwdriver head, such as a hexalobular, torx, 6-point and/or star screwdriver head and/or driver, and the cavity 458 may be of a corresponding geometry which permits the cavity 458 to removably receive the tip 652 and/or the polygonal end 658. In other examples, the tip 652 and the end 658 may be of any other corresponding geometry to the cavity 458 to permit the cavity 458 to removably receive the tip 652 and/or the polygonal end 658. In embodiments, when the tip 652 is engaged with the cavity 458, the first longitudinal axis 406 and the third longitudinal axis 656 may be aligned (e.g., overlap).

In some embodiments, the tip 652 may access the cavity 458 by being inserted into the implant 400 through the first opening 416 and further passing through the cannula 430. The tip 652 may be locked into, connected to, or otherwise secured within the cavity 458 via engagement of the threaded end 645 with the cannula 430. For example, when the tip 652 is inserted into the cavity 458, the handle 643 of the inserter, and thus also the member 644 including the threaded end 645, may be rotated to secure the threaded end 645 to the first inner surface 426. The knob 646, and thus also the rod 650, may then be rotated to transition the implant 400 from the first state 446 to the second state 448. In other embodiments, the handle 643 and the member 644 can be rotated to secure (e.g., removably secure) the threaded end 645 to the first inner surface 426 without also rotating the rod 650 and/or the knob 646.

The threaded end 645 may be unlocked, disconnected, or otherwise unsecured from the first inner surface 426 by a rotation of the handle 643 and member 644 in a reverse direction from that which it was initially rotated to engage the first inner surface 426. In some embodiments, the tip 652 may further be unlocked, disconnected, or otherwise unsecured from the cavity 458 following disengagement of the threaded end 645 from the first inner surface 426 as just described by moving the inserter 640 in a reverse direction from that in which the tip 652 was initially inserted into the cavity 458.

In some embodiments, the inserter 640 may be configured (e.g., sized, shaped and/or dimensioned) such that the knob 646, including the bar 647, and the rod 650 may advantageously spin, twist, turn and/or rotate independently from the handle 643 and member 644. For example, when the tip 652 is inserted into the cavity 458, the handle 643 and the member 644 may be rotated independently of the knob 646 and the rod 650 to secure the threaded end 645 to the first inner surface 426. Then, the knob 646 and the rod 650 may be rotated independently of the handle 643 and the member 644 to draw the hard tip 450 into the cannula 430 in order to transition the implant 400 from the first state 446 to the second state 448.

In some embodiments, the inserter 640 may be configured (e.g., sized, shaped and/or dimensioned) such that the knob 646, including the bar 647, and the rod 650 may advantageously spin, twist, turn and/or rotate in an opposite direction from the handle 643 and the member 644. For example, the direction in which the handle 643 and the member 644 are rotated to secure the threaded end 645 to the first inner surface 426 may be opposite from the direction in which the knob 646 and the rod 650 are rotated to transition the implant 400 from the first state 446 to the second state 448 (e.g., to draw the hard tip 450 into the cannula 430). Thus, in some embodiments, the threading of the first inner surface 426 may be in an opposite direction of the threading of the second inner surface 427 to facilitate this opposing rotation.

The independent and/or opposing rotations of the knob 646 and rod 650 as opposed to the handle 643 and member 644 allows for minimizing the risk of mistakes during performance of the method as described below. For example, there is a risk that the inserter 640 disconnects from the implant 400 during insertion into an incision, such as by the threaded end 645 inadvertently being disconnected from the first inner surface 426 of the implant 400, which may cause the implant 400 to be lost in the incision or otherwise dislocated from the inserter 640. By implementing reverse rotations of the knob 646 and the rod 650 as compared to the handle 643 and member 644, the chance of the threaded end 645 being prematurely, improperly and/or inadvertently disconnected from the first inner surface 426 during a transition of the implant 400 from the first state 446 to the second state 448 (e.g., during a rotation of the knob 464 and rod 650) is reduced, minimized or eliminated. Similarly, by implementing reverse rotations of the handle 643 and member 644 as compared to the knob 646 and rod 650, the chance that the implant 400 may be prematurely, improperly and/or inadvertently transitioned from the first state 446 to the second state 448 (e.g., during a rotation of the handle 643 and the rod 650) may be reduced, minimized or eliminated. The implementation of reverse rotations as just described thus helps to isolate some movements from other movements which are intended to occur at different points and/or times during performance of the surgical method, or which are intended to have different functions, as described below.

Turning to FIGS. 11-14, the system may further include the sleeve 200 configured (e.g., sized, shaped and/or dimensioned) to be inserted into a surgical incision, cut or wound. The sleeve 200 may further be configured (e.g., sized, shaped and/or dimensioned) to facilitate the insertion of various components, including the implant 400, into the cut, surgical dam, and/or bone. The sleeve 200 may include the body 201, the first end 202 having the first opening 208, and the second end 204 having the second opening 210. In some embodiments, the body 201 may be tubular, which may facilitate insertion of the sleeve 200, for example, into a grommet or surgical dam. The through-bore or channel 212 may extend through the body 201 from the first opening 208 to the second opening 210, such that the first opening 208 is in fluid communication with the second opening 210. In some embodiments which include a grommet, there may be no sleeve 200 and the method may instead proceed through an opening in the cut provided by the grommet, in which case the grommet may function as the sleeve. In such embodiments, the tendon may be pinned to the bone through some other means. For example, in some embodiments, there may be another anchor or implant (e.g., another of the implant 400, or some other implant or instrument) which is already holding the tendon to the bone, obviating the need to use the sleeve 200 for the same purpose when subsequently inserting the implant 400.

The first opening 208, the second opening 210, and/or the channel 212 may have a diameter which is larger than a largest width or a largest diameter of the implant 400 and/or the inserter 640 to permit the implant 400 to fit through (i.e., to be inserted into) the first opening 208 and the channel 212, and to exit the second opening 210 (i.e., to exit out of the channel 212), as described in greater detail below with respect to the method of the present disclosure. More specifically, the head 410 of the implant 400 may in some embodiments be the widest portion of the implant 400, and in such embodiments the head 410 may have a diameter that is smaller than the diameter of the first opening 208, the second opening 210, and the channel 212. In some embodiments, at least a portion of the body 201 may be transparent to assist with visualization of the implant 400 and various other instruments described herein as they pass into and/or through the channel 212. The transparent portion of the body 201 may be, for example, at least one window (not shown) along the length of the sleeve 200.

As shown in FIGS. 11-14, the sleeve 200 may include a collar 206 extending longitudinally along a portion of the body 201 and circumferentially around an outer surface 216 of the body 201. The collar 206 may permit a user (e.g., a surgeon) to ergonomically control the sleeve 200 during insertion into and removal from the cut or incision, as described in more detail below. In an example depicted in FIGS. 11-14, the collar 206 may be disposed towards the first end 202 of the sleeve 200, for example, between a midpoint of the sleeve 200 and the first end 202.

The second end 204 of the sleeve 200 may further include a sleeve tip 220 configured (e.g., sized, shaped and/or dimensioned) to hold a tendon in place relative to a bone during the process of inserting the implant 400 into the incision and/or the bone. The sleeve tip 220 may include the second opening 210. An edge 222 of the second opening 210 may include a plurality of prongs 226, 228 and a plurality of arches 230, 232. In an example shown in FIGS. 11-12, the plurality of prongs 226, 228 may include a first prong 226 and a second prong 228 positioned opposite (e.g., diametrically opposite or substantially opposite, depending on the embodiment) from each other along the edge 222. The plurality of arches 230, 232 may include a first arch 230 and a second arch 232 positioned opposite (e.g., diametrically opposite or substantially opposite, depending on the embodiment) from each other along the edge 222. The first arch 230 may connect the first prong 226 to the second prong 228 on a first side and the second arch 232 may connect the first prong 226 to the second prong 228 on a second side opposite the first side. Although the example embodiment shows the two prongs 226, 228 and the two arches 230, 232, alternative numbers of the prongs 226, 228 and the arches 230, 232 are also expressly contemplated.

The plurality of prongs 226, 228 may contact and/or engage a tendon or other soft tissue structure to pin and/or secure the tendon or soft tissue in place relative to a bone when the sleeve 200 is inserted into the cut or incision to contact at least a portion of the bone.

Importantly, the prongs 226, 228 may be configured (e.g., sized, shaped and/or dimensioned) to contact the tendon and hold the tendon in place such that a natural anatomic tensioning of the tendon (e.g., the biceps tendon) is maintained throughout the tenodesis procedure (i.e., during insertion of the implant 400 into the bone). Maintaining natural anatomic tensioning of the tendon may obviate the need for a surgeon to tenotomized and manually re-tension the tendon, which may in turn reduce and/or eliminate risks of negative surgical outcomes (e.g., pop-eye deformity).

As shown in FIGS. 11-13, the sleeve tip 220 may further include a measuring line and/or alignment indicator 224 located at a crown or a center of the first arch 230 and/or the second arch 232. The alignment indicator 223 may permit a surgeon or other medical professional or user to visualize various aspects of the method of the present disclosure. For example, the alignment indicator 224 may be used to determine whether certain components (e.g., the implant 400, the punch 322, the k-wire 320, etc.) are aligned in the desired position over the soft tissue (e.g., tendon) to be fixed to a bone. The alignment indicator 224 may provide an indication of a central position of various components as they pass through the sleeve 200, allowing for the user (e.g., surgeon) to visualize the engagement position with the soft tissue and bone. The alignment indicator 224 may further provide an indication of a rotational alignment of the sleeve 200 relative to the implant 400, or of the implant 400 relative to a tendon 672 and/or a bone 670, which may be valuable in situations where the head 410 of the implant 400 is a shape other than circular (e.g., ovular or polygonal), in which case the alignment of the head 410 may be of increased importance.

The sleeve 200 may be configured (e.g., sized, shaped and/or dimensioned) to removably receive the sleeve plug 214 in the channel 212, as shown in FIG. 20. When the sleeve 200 is initially inserted into the surgical cut, the channel 212 may be occupied or substantially occupied by the sleeve plug 214 such that fluid communication between the first opening 208 and the second opening 210 of the sleeve 200 may be fully or partially prevented, blocked, reduced, or otherwise obstructed. The presence of the sleeve plug 214 within the channel 212 may prevent unwanted matter, such as facia, fat, muscle, or other soft tissues, from clogging and/or contaminating the channel 212 when the sleeve 200 is inserted into the cut. The sleeve plug 214 may be configured (e.g., sized, shaped and/or dimensioned) to be removed from the sleeve 200 to expose the channel 212 and strengthen or otherwise reestablish fluid communication between the first opening 208 and the second opening 210 of the sleeve 200. Other components, including the implant 400 and the inserter 640, may then be inserted into and/or through the channel 212 (i.e., into and/or out of the first opening 208 and/or the second opening 210), as described in more detail below.

As shown in FIGS. 48 and 50, the sleeve 200 may be configured (e.g., sized, shaped and/or dimensioned) to removably receive the implant 400 and/or at least a portion of the inserter 640. For example, following engagement of the threaded end 645 of the member 644 with the first inner surface 426 of the cannula 430, the implant 400 may be engaged with or otherwise attached to the inserter 640, such as at the second end 641 of the inserter 640. In such a configuration, the implant 400, at least a portion of the inserter which includes the tip 652, and/or at least a portion of the member 644 may be configured (e.g., sized, shaped and/or dimensioned) to be received in the channel 212 of the sleeve 200. The member 644 may thus have a length which is sufficiently long to allow for the implant 400, while attached to the second end of the inserter 640, to be inserted into the first opening 208, through the channel 212, and out of the second opening 210 to reach the desired placement for the implant 400 (e.g., to reach the soft tissue and/or bone). The implant 400 and the portion of the member 644 which is received in the channel 212 may thus have a largest diameter, which is smaller than a smallest diameter of the channel 212, to ensure the implant 400 can be properly inserted into the bone through the channel 212.

In further embodiments of the system, for example, where the cannula 430 extends through the hard tip 450 and the hard tip 450 does not include the point 454 (FIG. 49), the system may further include the punch 322, the k-wire 320 and/or the k-wire cap 300. The punch 322, the k-wire 320 and/or the k-wire cap 300 may be present in the system where the cannula 430 extends through the hard tip 450 and instruments other than the hard tip 450 are used to create a hole in the bone into which the implant 400 may be inserted. Thus, in embodiments, one such instrument which may be inserted into the channel 212 of the sleeve 200 during the method as described below is the punch 322, as shown in FIGS. 22-25.

The punch 322 may include a handle 324 and a first portion 323 extending from the handle 324 to the punch tip 326, as shown in FIG. 23. The handle 324 may function as a gripping point for ergonomically controlling the punch 322, such as when the punch 322 is being inserted into the channel 212. In some embodiments, the handle 324 may further provide a surface which allows for the punch 322 to be back-tapped out of the surgical cut and/or the bone, as shown in FIG. 25. The punch tip 326 may be made of a hard material, such as metal, to permit the punch tip 326 to be driven into the bone to the first predetermined depth in response to the first impact applied (directly or indirectly) to the punch 322. The punch 322 may also be configured (e.g., sized, shaped and/or dimensioned) to receive the k-wire 320, wherein the punch tip 326 may be hollow and/or include a channel to permit the k-wire 320 to extend out of and/or beyond the punch tip 326 and into the bone to the second predetermined depth in response to the second impact. The first impact and the second impact are described in more detail below with respect to the method of the present disclosure.

The first portion 323 of the punch 322 may have a diameter which is smaller than the diameter of the first opening 208, the second opening 210, and/or the channel 212 to permit at least the first portion of the tip 326 to be inserted into the channel 212 as shown in FIGS. 22-23 and 25. When the punch 322 is inserted into the channel 212 of the sleeve 200, the punch tip 326 may extend beyond (i.e., exit) the second opening 210 of the sleeve 200 to contact the soft tissue (e.g., a tendon) and/or the bone. The first portion 323 may have a predetermined length which is longer than a length of the sleeve 200 from the first end 202 to the second end 204, such that a first gap 328 is left between the first end 202 of the sleeve 200 and the handle 324 of the punch 322 when the punch 322 is inserted into the channel 212 of the sleeve 200, as can be seen in FIGS. 22-23.

The first gap 328 between the handle 324 of the punch 322 and the first end 202 of the sleeve 200 may have a distance and/or length. The distance and/or length may be equal to the first predetermined depth to act as a stop for the punch tip 326. Where the first gap 328 acts as a stop, the first gap 328 prevents the punch tip 326 from being driven undesirably into the bone to a depth which is further than the first predetermined depth. For example, the first impact applied to the punch 322 may drive the first portion 323 further into the channel 212 such that the punch handle 324 meets the first end 202 of the sleeve 200, causing the punch tip 326 to puncture through the tendon and/or into the bone to the first predetermined depth, as shown in FIGS. 23-24. Once the punch tip 326 is driven through the tendon and/or into the bone to the first predetermined depth, the handle 324 may then be back-tapped to remove the punch tip 326 from the tendon and/or the bone (and/or to remove the punch 322 from the channel 212). Back-tapping includes tapping, hammering or otherwise impacting the punch 322 (e.g., the handle 324) in a direction opposite of inserting the punch 322 into the channel 212. Removing the punch 322, and therefore also the punch tip 326, from the channel 212 may leave a cavity in the bone 350 which is configured (e.g., sized, shaped and/or dimensioned) to receive the implant 400.

The first impact may be applied to the punch 322 and the k-wire 320, for example directly or indirectly through the k-wire 320 and/or the k-wire cap 300. In an example shown in FIGS. 22-25, the k-wire 320 may be inserted into and/or through the channel 212 of the sleeve 200 and may further be inserted into a second channel (not shown) extending through the punch 322 to contact and/or extend into a portion of the punch tip 326. As shown in FIG. 24, the exposed end 321 of the k-wire 320 may be configured (e.g., sized, shaped and/or dimensioned) to receive the k-wire cap 300 and may extend outside of the punch 322 and/or the sleeve 200 in a direction opposite of that in which the sleeve 200 is inserted into the cut (i.e., in a direction from the second end 204 of the sleeve 200 toward the first end 202 of the sleeve 200).

Turning now to FIGS. 15-19, the k-wire cap 300 may include the first end 302 having the first opening 308 to the first cavity or long cavity 312, the second end 304 having the second opening 314 to the second cavity or short cavity 318, and the body 306 extending from the first end 302 to the second end 304. The body 306 of the k-wire cap 300 may include the circumferential ribs or protrusions 307 to assist in gripping and/or ergonomically controlling the k-wire cap 300. The long cavity 312 may extend from the first opening 308 toward the second end 304. In addition, the long cavity 312 may extend from the first opening 308 toward the short cavity 318. The short cavity 318 may extend from the second opening 314 toward the first end 302. In addition, the short cavity 318 may extend from the second opening 314 toward the long cavity 312. In an embodiment shown in FIGS. 17-19, the long cavity 312 and the short cavity 318 may extend towards each other along the second longitudinal axis 332 extending through a center of the k-wire cap 300 from the first end 302 to the second end 304. In an example, the long cavity 312 may be centered within the first end 302 and the short cavity 318 may be centered within the second end 304. The first end 302 and the second end 304 of the k-wire cap 300 may be substantially flat or planar to provide a surface on which the first impact and the second impact may be applied (directly or indirectly) to the k-wire cap 300. When the k-wire cap 300 is impacted, the punch 322 and/or the k-wire 320 may be indirectly impacted, as described above.

The long cavity 312 and/or the short cavity 318 may be configured (e.g., sized, shaped and/or dimensioned) to receive at least a portion of the k-wire 320 including the exposed end 321. When at least a portion of the k-wire 320 is received in the channel 212 and the exposed end 321 is received in either the long cavity 312 or the short cavity 318, the k-wire 320 may align the k-wire cap 300 with the punch 322 and/or the sleeve 200. For example, the long cavity 312 may be configured (e.g., sized, shaped and/or dimensioned) to receive (e.g., slide over) the exposed end 321 such that the first end 302 of the k-wire cap 300 contacts the punch 322 when the k-wire 320 is received in the long cavity 312, as shown in FIG. 23. Similarly, the short cavity 318 may be configured (e.g., sized, shaped and/or dimensioned) to receive (i.e., slide over) the exposed end 321 such that the k-wire cap 300 is aligned with the punch 322 and/or the sleeve 200. In such an example, the k-wire 320 secures the k-wire cap 300 in place relative to the punch 322 such that the components are aligned, reducing the risk that the first impact may be applied incorrectly. The alignment indicator 224 may assist in determining whether the components are properly aligned with each other and/or with the soft tissue and/or bone of the patient.

In an embodiment shown in FIG. 19, the long cavity 312 may include the first angled, tapered, or beveled surface or countersink 310 extending from the first opening 308 toward the second end 304 such that the k-wire 320 may be guided by the first countersink 310 when inserted into the long cavity 312. Similarly, the short cavity 318 may include the second angled, tapered, or beveled surface or countersink 316 extending from the second opening 314 toward the first end 302 such that the k-wire 320 may be guided by the second countersink 316 when inserted into the short cavity 318. The first countersink 310 and the second countersink 316 may thereby facilitate or otherwise make easier the insertion of the k-wire 320 into the long cavity 312 and/or the short cavity 318.

The long cavity 312 of the k-wire cap 300 may be configured (e.g., sized, shaped and/or dimensioned) to facilitate the first impact applied to the punch 322 and/or the k-wire 320. For example, when the k-wire 320 is received in the long cavity 312 such that the first end 302 of the k-wire cap 300 contacts the punch 322 as described above, the k-wire cap 300 may work in tandem with the first gap 328 to ensure the punch 322 and/or the k-wire 320 is driven into and/or through the tendon and/or the bone to the first predetermined depth, as shown in FIG. 23. The first impact may be applied to the second end 304 of the k-wire cap 300 such that the force transfers through the k-wire cap 300 to the punch 322 and/or the k-wire 320, which forces the punch 322 further into the channel 212 of the sleeve 200 such that the first gap 328 is closed. The first impact may thus allow for the handle 324 of the punch 322 to meet and/or contact the first end 202 of the sleeve 200. When the first gap 328 is closed, the punch tip 326 may be driven and/or forced into the bone to the first predetermined depth. The handle 324 may prevent the first impact from driving the punch tip 326 beyond the first predetermined depth by acting as a stop preventing further movement of the punch 322 and/or the punch tip 326 in the same direction that the first impact is applied, for example, in the direction of the sleeve 200 being inserted into the cut. In some embodiments, the first impact may instead be multiple first impacts which progressively close the first gap 328.

The short cavity 318 may be configured to facilitate the second impact applied to the k-wire 320. As shown in FIG. 24, when the k-wire 320 is received in the short cavity 318, the second end 304 of the k-wire cap 300 may face the punch 322 and a second gap 330 may exist between the k-wire cap 300 and the punch 322. The second gap 330 may be a distance which is equal to the second predetermined depth, which is further (e.g., deeper) than the first predetermined depth. The second impact may be applied to the first end 302 of the k-wire cap 300 such that the force transfers through the k-wire cap 300 to the k-wire 320, thereby driving and/or forcing the k-wire 320 into the bone to the second predetermined depth and closing the second gap 330 such that the second end 304 of the k-wire cap 300 contacts the punch 322. The handle 324 may prevent the second impact from driving the k-wire 320 beyond the second predetermined depth by acting as a stop preventing further movement of the k-wire cap 300 beyond the point of contact between the k-wire cap 300 and the punch 322 in the same direction that the second impact is applied, for example, in the direction of the sleeve 200 being inserted into the cut. In some embodiments, the second impact may instead be multiple second impacts which progressively close the second gap 330.

To ensure that the k-wire 320 and/or the punch 322 (i.e., the punch tip 326) has/have been driven into the bone to the proper distance, for example, to the first predetermined depth and/or the second predetermined depth, in response to the first impact and/or the second impact, respectively, the bone and/or the area surrounding the bone which includes the tendon may be imaged and/or scanned (e.g., CT scanned, x-rayed, etc.) and evaluated.

Another method of using the implant 400 for reattachment of soft tissue to a pre-selected bony structure includes utilizing some or all of the components of the system described above to perform a tenodesis procedure (e.g., to repair a tendon). The method may reduce the number of steps required for surgical repair of a tendon whilst maintaining (natural) anatomic tensioning thereof. Although the method has been described with respect to the biceps tendon, it is understood and expressly contemplated that the implant 100 may be used with any tendon or like soft tissue structure. A tenodesis according to the method disclosed herein may therefore avoid the need to tenotomized the tendon and/or retrieve it, for example, using whip stitching outside the body. Maintaining anatomic tensioning during a tenodesis procedure according to the method disclosed herein may further eliminate the need for a surgeon to perform the challenging and non-reproducible step of precisely re-tensioning the tendon. The present method may therefore reduce and/or eliminate the risk of the tendon being improperly secured to the bone and may further reduce and/or eliminate the risk of various side effects and/or malformities which may result from a tenotomy and/or a tenodesis, such as pop-eye deformity.

The method will be described below with respect to a biceps tenodesis procedure, however, it will be obvious to those skilled in the art that aspects of the present disclosure may be practiced in other areas of the body, for example, to repair other tendons. The method may include multiple variations depending on the quality and/or hardness of the bone or other factors. The bone may be any bone suitable for soft tissue fixation. In an example shown in FIGS. 20-21 and 44-48, the method may utilize the implant 400 including the hard tip 450 and the inserter 640 to pierce a bone 350. In an example shown in FIGS. 20-26 and 49-50, the method may utilize the punch 322, including the punch tip 326, to pierce the bone 670 to create a cavity in the bone 670 for inserting and/or securing the implant 400. The bone 670 may be, for example, a cortical or compact bone.

Turning to FIGS. 20-21, the method may include inserting the sleeve 200 into a wound, incision, or cut (not shown), the sleeve 200 having removably received the sleeve plug 214 within the channel 212 of the sleeve 200, such that the sleeve tip 220 and/or the sleeve plug 214 contacts a tendon 672 and/or the bone 670. In some embodiments, the sleeve plug 214 may occupy the channel 212 during insertion into the cut to prevent matter, such as soft tissue, from getting trapped in the channel 212, and to prevent pressurized fluid from the cut being expelled from the channel 212. In addition, the collar 206 may be gripped to assist in inserting, removing and/or otherwise ergonomically controlling the sleeve 200.

In other embodiments, prior to inserting the sleeve 200 into the cut, a grommet or surgical dam (not shown) may be inserted into the cut. The grommet or surgical dam may be a standard grommet or surgical dam of the kind traditionally used in surgery to provide an opening or other access point through which a surgical procedure (e.g., a biceps tenodesis) may take place. Thus, throughout the method as described herein, any actions which are performed during the method may take place through or in the absence of a grommet or surgical dam. The grommet may be, for example, self-sealing.

The sleeve 200 and/or the sleeve plug 214 may then be used to locate the tendon 672. Next, the sleeve tip 220 may engage the tendon 672 to capture and/or otherwise hold the tendon 672 in place relative to the bone 670. Preferably, the sleeve 200 and/or the sleeve plug 214 locate and/or contact the tendon 672 and/or the bone 670 substantially at an expected implantation site for the implant 400. The contact point of the sleeve 200 and/or the sleeve plug 214 may be, for example, at a point along the bone 670 and/or the tendon 672 at which the tendon 672 is to be compressed to the bone 670 by the implant 400. The contact point may be selected such that the implant 400 may later be inserted through the channel 212 to pin the tendon 672 to the bone 670 without needing to reposition the sleeve 200. The contact point may thus preferably be a point at which pinning the tendon 672 to the bone 670 best maintains natural anatomic tensioning of the tendon 672. The sleeve plug 214 may then be removed from the channel 212 to strengthen, establish or re-establish fluid communication between the first opening 208 and the second opening 210. The channel 212 may then be utilized for subsequent aspects of the method (e.g., subsequent aspects of the surgical procedure).

The sleeve tip 220 may hold the tendon 672 in place relative to the bone 670 via the first prong 225 and/or the second prong 228. Preferably, the sleeve tip 220 may contact the tendon 672 such that the natural anatomic tensioning of the tendon 672 is undisturbed (i.e., is maintained). The sleeve tip 220 may pin the tendon 672 to the bone 670 throughout many aspects of the procedure, for example, until the implant 400 is fully seated or inserted as described in more detail below.

In some embodiments, the implant 400 may be prepared to be inserted into the cut, for example, through the channel 212 following the pinning of the tendon 672 in place via the sleeve tip 220 as just described above. Preparing the implant 400 may occur at any point prior to insertion of the implant 400 into the cut. For example, the implant 400 may be prepared to be inserted prior to the cut being made in a patient. Alternatively, the implant 400 may be prepared to be inserted into the cut during the insertion of the sleeve 200, such as by a surgical assistant. Further, the implant 400 may be prepared to be inserted into the cut following the placement of the sleeve 200 (e.g., the pinning of the tendon 672 to the bone 670 as described above).

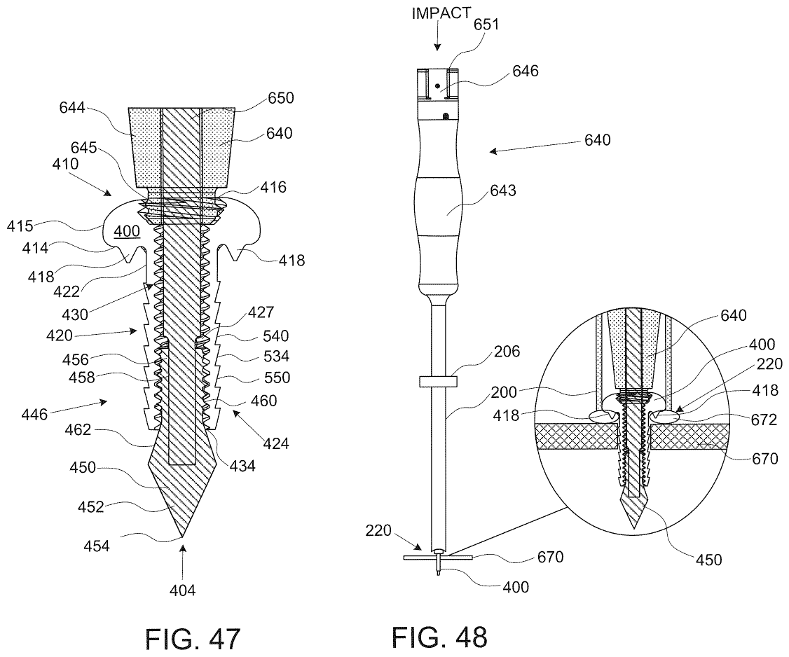

The method may also include preparing the implant 400 to be inserted into the cut which includes connecting the implant 400 to the inserter 640, as shown in FIG. 47. For example, the tip 652 of the rod 650 of the inserter 640 may be inserted into and/or removably received in the cavity 458 of the hard tip 450 of the implant 400, such as by being inserted through the first opening 416 and/or the cannula 430. Then, the tip 652 may be locked into, connected to, or otherwise secured within the cavity 458 via engagement of the threaded end 645 of the inserter 640 with the cannula 430. For example, when the tip 652 has been inserted into the cavity 458, the handle 643 of the inserter 640, and thus also the member 644 including the threaded end 645, may be twisted, turned, rotated, and/or spun to secure the threaded end 645 to the first inner surface 426. The securing of the threaded end 645 to the first inner surface 426 may prevent or minimize the risk of the implant 400 prematurely and/or undesirable detaching from the inserter 640 during insertion of the implant 400 into the cut (and/or a grommet or surgical dam), which could result in the implant being lost and subsequently needing to be retrieved from the cut.

Once secured to the inserter 640, the implant 400 may be in condition to be inserted into the cut, as shown in FIGS. 47-48. The method may also include inserting the implant 400 into the cut, which may include inserting the implant 400 (which may be attached to the threaded end 645 of the inserter 640) into the sleeve 200. For example, the implant 400 and at least a portion of the inserter 640 which includes the threaded end 645 and the tip 652 may be inserted into the channel 212 of the sleeve through the first opening 208 in a direction from the first opening 208 towards the second opening 210. The implant 400 may travel through the channel 212 until reaching and/or exiting the second opening 210 to reach the desired placement or substantially the desired placement for the implant 400 (e.g., to reach the tendon 672 and/or the bone 670).

Once the implant 400 has reached the desired placement or substantially the desired placement, the implant 400 may be seated or partially seated in the bone 670. For example, when the implant 400 has reached the desired placement, the hard tip 450 may contact the tendon 672 and/or the bone 670, and the point or tip 454 of the hard tip 450 may be positioned to be driven through the tendon 672 and/or the bone 670 to secure the implant 400 in place. In some embodiments, the point 454 may be driven through the tendon 672 and/or the bone 670 by way of a first impact applied to the inserter 640. For example, the first impact may be applied to the flat end face 651 of the inserter 640 (and thus, indirectly to the implant 400) in a direction substantially from the first end 641 of the inserter 640 towards the second end 642 of the inserter 640 to drive the hard tip 450 through the tendon 672 and into the bone 670. In some embodiments, the head 410 of the implant 400 may have a diameter which is larger than a second diameter of a hole in the bone 670 formed by the driving of the hard tip 450 into the bone 670 as just described., such that the head 410 is too wide to fit through the hole in the bone 670 formed by the hard tip 450 as just described. The head 410 may therefore act as a stop to prevent the implant 400 from being inserted into the bone 670 to an undesirable depth.

Preferably, the implant 400 is driven into the bone 670 to a distance at which the first venting hole 440 and/or the second venting hole 441 are aligned with a space between the tendon 672 and the bone 670 to facilitate the delivery of healing agents thereto. The desired positioning of the venting holes 440, 441 may influence the placement thereof along the stem 420 when the implant 400 is manufactured. Further, it is preferable that the implant 400 be driven into the bone such that the second opening 434 is in fluid communication with an intramedullary region of the bone 670 to permit passage of biological agents (e.g., healing agents) from the intramedullary region of the bone to the space between the tendon 672 and the bone 670, for instance, via the cannula 430.

Once the implant 400 has been driven through the tendon 672 and into the bone 670 (e.g., at the desired placement to pin the tendon 672 to the bone 670), the implant 400 may then need to be secured in place. In some embodiments, once the implant 400 has been inserted in the bone 670, the implant 400 may be transitioned from the first state 446 to the second state 448 to secure or fully seat the implant 400 in place. To transition the implant 400 from the first state 446 to the second state 448 the user may have to twist, turn, rotate and/or spin the knob 646 of the inserter 640. For example, at this point the tip 652 of the inserter 640 may be located in the cavity 458 of the hard tip 450 as described above. Thus, the knob 646, which is connected to the rod 650, may be rotated to rotate the tip 652 of the rod 650 within the cavity 458, causing the hard tip 450 to rotate.

As the hard tip 450 rotates, the second stem 465 may progress along the threading of the second inner surface 427 as shown in FIG. 45, such that the hard tip 450 is drawn into the cannula 430 (e.g., in a direction from the second end 404 of the implant 400 towards the first end 402 of the implant 400). As the hard tip 450 advances through the cannula 430, the tapered portion 462 of the second head 452 of the hard tip 450 may apply a force and/or pressure to the second opening 434, which may cause the cannula 430 to break and/or snap, for example, at the first breakaway point 442 and/or the second breakaway point 443. The breaking and/or snapping of the breakaway points 442, 443 may make a pop which may be heard or felt (e.g., through vibrations) by the user (e.g., a surgeon), which may provide an indicator to the surgeon that the implant 400 has begun its transition from the first state 446 to the second state 448.

In some embodiments, the breaking or snapping of the breakaway points 442, 443 may separate the stem 420 of the implant 400 into the first wing 431 and the second wing 432. In other embodiments, the knob 646 may continue to be rotated to continue advancing the hard tip 450 into the cannula 430 (e.g., towards the head 410), which may cause the wings 431, 432 to separate or otherwise move away from each other. The movement of the wings 431, 432 relative to each other may be in opposite directions or substantially opposite directions. The wings 431, 432 may move away from each other about the venting holes 440, 441, such that the portions of the wings 431, 432 which are closer to the second opening 434 move a further distance than the portions of the wings 431, 432 which are closer to the venting holes 440, 441.

As the wings 431, 432 move away from each other, the stem 420 may widen within the bone 670 such that the stem 420 becomes wider than the hole in the bone 670 made by the hard tip 450, securing the implant 400 in place relative to the bone 670, as shown in FIG. 46.

This movement of the wings 431, 432 away from each other is also referred to herein as the transitioning of the implant 400 from the first state 446 to the second state 448. Said another way, once the wings 431, 432 are sufficiently expanded to prevent pullout of the implant 400, the implant 400 may be said to be in the second state 448. In the second state 448, the ridges 424 may assist in securing the implant 400 in place in the bone 670 by resisting pullout of the implant 400 in a direction opposite that which the implant 400 was inserted.

In some embodiments, the desired objective of pinning the tendon 672 to the bone 670 may be said to have been accomplished once the implant 400 is in the second state 448 (e.g., following expansion of the wings 431, 432). In other embodiments, pinning the tendon 672 to the bone 670 may include inserting or otherwise implanting one or more other implants (e.g., one or more additional implants which are identical to or similar to the implant 400) along the tendon 672 and/or bone 670, such as longitudinally along the bone and/or along the length or longest dimension of the bone. In such embodiments, the desired objective of pinning the tendon 672 to the bone 670 may be said to have been accomplished once each implant of the implants have been inserted into the bone 670 and transitioned from its respective first state (e.g., the first state 446) to its respective second state (e.g., the second state 448). At this point, a user (e.g., a surgeon) may proceed to remove instruments and/or components such as the inserter 640, the sleeve 200, the grommet (not shown), or any other pieces present in the cut.

In some embodiments, prior to removing some or all relevant instruments and/or components from the cut, sutures may be used to secure or further secure the implant 400. For example, one or more sutures may be anchored to the tendon and/or the bone via the implant 400 (e.g., via compression by the bottom surface 414 of the head 410, or by being driven into the bone along with the implant 400 as described above). The one or more sutures may be, for example, used to further secure the implant 400 in place, such as by suturing the implant 400 to surrounding tissues (e.g., the tendon 672). In some embodiments, the one or more sutures may be used to repair damage to the tendon 672 such as by suturing portions (e.g., bundles of tendon fibers of the tendon 672) of the tendon 672 together, for example, by whip stitching or mattress stitching, among others, in order to supplement the longitudinal strength of the tendon 672. In some embodiments, the supplemental suturing of the tendon 672 may enhance the hold of the implant 400 when one or more of the protrusions 418 pierce and/or compress the tendon 672 to the bone 670. In some embodiments, one or more suture(s) may already be present in the implant 400 when the implant 400 is inserted into the bone 670. In other embodiments, the one or more suture(s) may be inserted after the implant 400 has already been inserted into the bone 670.

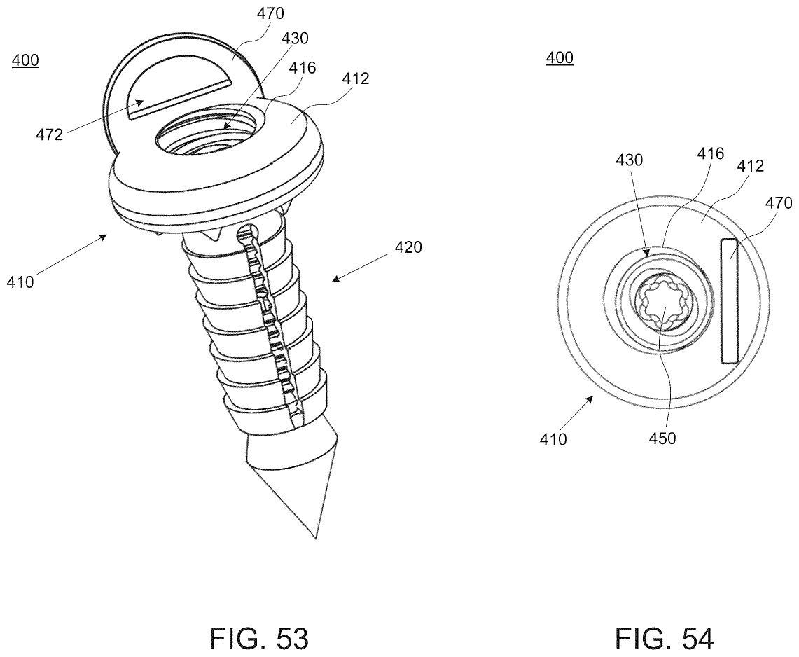

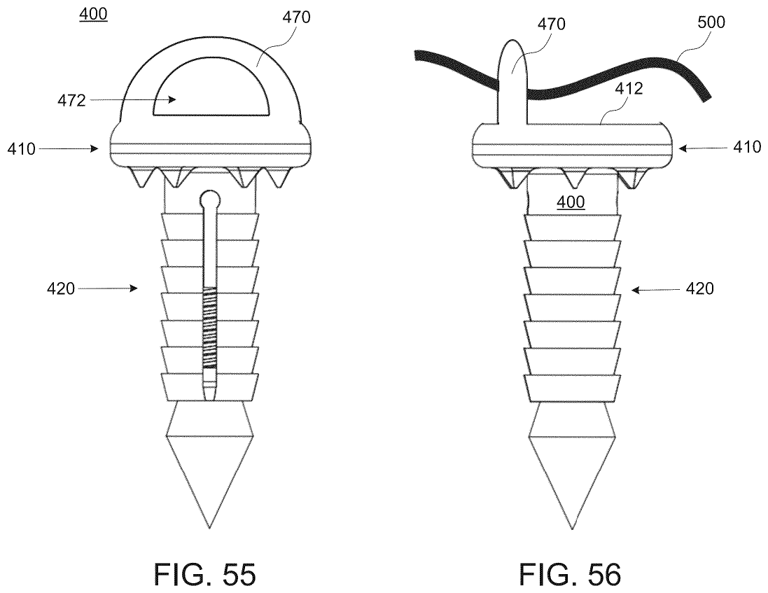

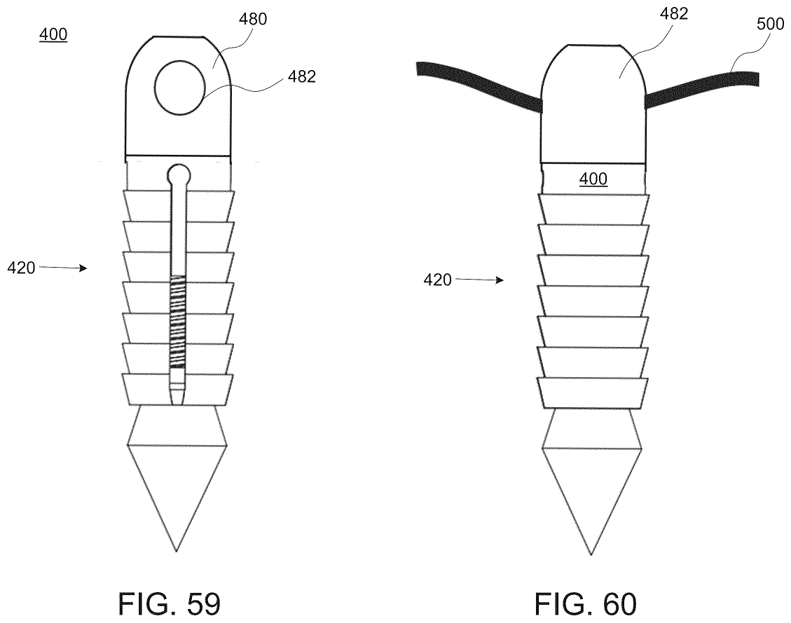

While sutures can be used to secure or further secure any embodiment(s) of the implant 400 described above, embodiments of the implant 400 may also be employed which include a suture passage specifically designed to accommodate one or more suture(s), examples of which are shown in FIGS. 53-60. Embodiments of the implant 400 which include a suture passage are described below in detail and may be used during the method as described above. Aspects of the embodiments discussed below and the uses and/or functions thereof during performance of the method for using the implant 400, are described in detail above and thus will not be described again for brevity sake. Aspects are described as if only a single suture is used with a suture passage, but it is explicitly contemplated that a suture passage could be used with multiple sutures simultaneously. In addition, it is explicitly contemplated that the implant 400 may include more than one suture passage for use with one or more suture(s).

In some embodiments, the head 410 of the implant 400 may include a suture passage 470, as shown in FIGS. 53-56. In embodiments, the suture passage 470 may provide an additional means by which the tendon 672 may be fixated by one or more suture(s) 500 to the bone 670 and/or relative to the implant 400. In some such embodiments, the suture passage 470 may extend from the head 410, such as from the top surface 412 of the head 410, for example. In an embodiment, the suture passage 470 may be a frame defining at least one hole 472 (e.g., at least one through-hole) through which a suture 500 may be threaded or through which the suture 500 may otherwise pass or extend, as shown in FIG. 56. In some embodiments, the suture 500 may be threaded through the hole 472 when the implant 400 is inserted into the bone 670. In other embodiments, the suture 500 may be threaded through the hole 472 after the implant 400 is inserted into the bone 670. In order to maintain a rotational alignment of the implant 400, the suture 500, threaded through the hole 472, may be pulled on and/or tensed while the implant 400 is transitioned from the first state 446 to the second state 448. Following insertion of the implant 400, the suture 500 may be used as described above to hold and/or supplement the strength of the tendon 672.

As shown in FIGS. 53-54, the suture passage 470 may be offset from the first opening 416 of the head 410 so as not to obstruct access to the cannula 430 (e.g., access by the inserter 640 necessary to transition the implant 400 from the first state 446 to the second state 448). While the hole 472 of the suture passage 470, shown in FIGS. 53-56, is depicted as being bound by a half-circle shape (e.g., an arc connected by a straight line), the hole 472 may be of any suitable shape to accommodate the suture 500, including being totally rounded (e.g., circular or ovular) or a polygonal shape.

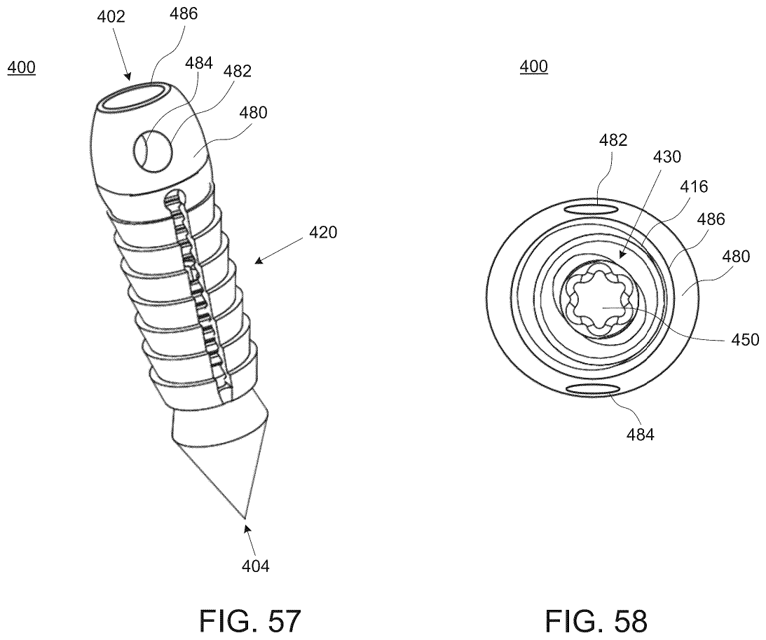

In other embodiments, the implant 400 may include an alternative suture passage 480 and no head, as shown in FIGS. 57-60. Said another way, in some embodiments, the implant 400 may include the suture passage 480 in place of and/or instead of the head 410. In some such embodiments, the suture passage 480 may extend from the first end 402 of the implant 400 to the stem 420, and the stem 420 may extend from the suture passage 480 to the second end 404 of the implant 400. In such embodiments, the first opening 416 of the cannula 430 may be located at an end of the stem 420 which is closest to the first end 402, for example, near or at the location where the stem 420 connects to the suture passage 480.

In some such embodiments, the suture passage 480 may include a first hole 482 and/or a second hole 484 through which the suture 500 may be threaded, or through which the suture 500 may otherwise pass or extend, as shown in FIG. 60. The first hole 482 and the second hole 484 are shown as being rounded, circular, or ovular, but it is explicitly contemplated that the first hole 482 and/or the second hole 484 may be of any suitable shape to accommodate the suture 500. In some such embodiments, the first hole 482 and the second hole 484 may be aligned with each other about a circumference of the suture passage 480. In other embodiments, the first hole 482 and the second hole 484 may be offset about the circumference of the suture passage 480, for example, to ensure the suture 500 is advantageously offset with respect to the first opening 416. This may ensure that the suture 500 does not obstruct the inserter 640 from being inserted into the cannula 430 when the suture 500 is threaded through the first hole 482 and/or the second hole 484.

In either case, the inserter 640 may in some embodiments optionally include a slot (e.g., in the tip 652 of the inserter 640) (not shown) to receive the suture 500, such that when the tip 652 of the inserter 640 is rotated within the cavity 458 as described above, the suture 500 is not disturbed by the transition of the implant 400 from the first state 446 to the second state 448. For example, the slot in the inserter may prevent the suture 500 from becoming wound around the inserter 640. In some embodiments, the suture 500 may be pressed by the inserter 640 against the second inner surface 426 of the stem 420, such that when the hard tip 450 is drawn up into the cannula 430 the suture 500 is held and/or locked in place between the hard tip 450 and the second inner surface 426 (e.g., by compression). In order to maintain a rotational alignment of the implant 400, the suture 500, threaded through the first hole 482 and/or the second hole 484, may be pulled on and/or tensed while the implant 400 is transitioned from the first state 446 to the second state 448. Following insertion of the implant 400, the suture 500 may be used as described above to hold and/or supplement the strength of the tendon 672.

As shown in FIGS. 57-58, the suture passage 480 may partially enclose, surround or encapsulate the first opening 416 of the cannula 430. The suture passage 480 may thus further include a third hole 486, for example, at the first end 402 of the implant 400 (e.g., on an opposite side of the suture passage 480 from the stem 420). The third hole 486 may also be aligned with the first opening 416 of the cannula 430 and may further have a radius which is the same as or larger than that of the first opening 416, as shown in FIG. 58. The sizing and alignment of the third hole 486 relative to the first opening 416 may permit the inserter 640 to be inserted through both the third hole 486 and the first opening 416 of the cannula 430 to reach the cavity 458 of the hard tip 450. While the embodiment shown in FIGS. 57-60 is depicted as having a flat top at the first end 402 (e.g., the end which features the third hole 486), it is explicitly contemplated that the suture passage 480 may have a rounded top (e.g., a dome) or may be of any other shape, so long as at least the first hole 482 and/or the second hole 484 permit passage of the suture 500, and so long as the third hole 486 permits the inserter 640 to access the cannula 430 as just described.

There may be embodiments (not shown) in which the suture passage 480 includes only the first hole 482 and the third hole 486 and no second hole 484. Similarly, there may also be embodiments in which the suture passage 480 includes the second hole 484 and the third hole 486 and no first hole 482. In these embodiments, the suture 500 may be threaded through the first hole 482 and the third hole 486 or through the second hole 484 and the third hole 486. Also in these embodiments, the suture 500 may be inserted into the third hole 486 as described above before, after, or at the same time as the inserter 640 is inserted into the third hole 486. In some such embodiments, the third hole 486 may have a slightly enlarged radius to accommodate both the suture 500 and the inserter 640 simultaneously during performance of the method. Alternatively, the inserter 640 may contain a recess into a side of the inserter 640 or a through hole extending through the inserter 640 for receiving the suture 500 that allows for the suture 500 to pass through the third hole 486 without enlarging the radius of the third hole 486.

In some embodiments, the suture passage 480 may have a radius which is equal to or less than a largest radius of the stem 420, such that the implant 400, including the suture passage 480, may be driven below the surface of the bone 670 during implantation. In such embodiments, the implant 400 may be entirely within the bone 670, and only the suture 500 (or sutures 500, in some embodiments) may extend outside of the bone 670. In such embodiments, the suture 500 may be used to secure the tendon 672 to the bone 670 and/or relative to the implant 400. Driving the implant 400 completely into the bone 670 may be particularly advantageous where there is not space in the area(s) surrounding the surgical site to accommodate a hard implant component. Driving the implant 400 completely into the bone 670 may thus reduce irritation, discomfort, and/or pain felt by a patient following a surgical procedure, including a tenodesis and other procedures.

In some embodiments, completing the surgical procedure may include checking the final positioning of the implant 400 and/or removing instruments and/or components from the cut. For example, completing the surgical procedure may include confirming that the implant 400 has been securely inserted into the bone 670 at the desired angle and/or positioning (e.g., on a center of an axis of the tendon 672), and/or that the tendon 672 is indeed secured to the bone 670 as intended.

In some embodiments, completing the surgical procedure may further include detaching the inserter 640 from the implant 400 and removing the inserter 640 from the cut. In some embodiments, the user may remove the inserter 640 from the cut by disengaging the threaded end 645 of the inserter 640 from the first inner surface 426 of the implant 400. The user may rotate the handle 643 of the inserter 640 to unlock, disconnect, disengage or otherwise unsecure the threaded end 645 from the first inner surface 426, and thus disconnect the inserter 640 from the implant 400. In some embodiments, to disconnect the threaded end 645 from the first inner surface 426, the user may rotate the handle 643 in a direction opposite of that in which the knob 646 was turned to transition the implant 400 from the first state 446 to the second state 448. For example, the handle 643 may be inserted into and/or engaged with the implant 400 by applying a clockwise rotation to the handle 643, while the handle 643 may be removed and/or disengaged from the implant 400 by applying a counter-clockwise rotation to the implant 400. In doing so, the user may hold the knob 646 steady such that the handle 643 and member 644 (including the threaded end 645) rotate independently of the knob 646 and the rod 650. Once disconnected, the inserter 640 may be removed from the cut in a direction opposite that which the inserter 640 was inserted as described above. This movement of the inserter 640 out of the cut may also pull the tip 652 of the rod 650 out of the cavity 458. The remaining components, such as the sleeve 200, may simply be moved in a direction opposite of that which they were inserted into the cut to be removed therefrom. The cut may then be closed using conventional methods (e.g., stitching) to complete the procedure.

In alternative embodiments, such as where the cannula 430 extends through the hard tip 450, there is no point 454 (FIG. 49) on the implant 400, the method may further include the k-wire 320, the k-wire cap 300, and/or the punch 322. In some embodiments, these components may be utilized following the insertion of the sleeve 200 into the cut and the removal of the sleeve plug 214 therefrom. Referring now to FIG. 22, once the sleeve plug 214 has been removed from the sleeve 200 to expose the channel 212, the punch 322 and/or the k-wire 320 may be inserted into the channel 212.

The punch 322 may be configured (e.g., sized, shaped and/or dimensioned) such that the first portion 323 thereof may slidingly engage (e.g., is capable of being inserted into) and/or otherwise removable received in the channel 212. The first portion 323 preferably has a length which is longer than the length of the channel 212 such that the punch tip 325 extends beyond the sleeve tip 220 to contact the bone 670 and/or the tendon 672 while leaving the first gap 328 between the punch handle 324 and the sleeve 200. As noted above, the first gap 328 may have a length which is equal to the first predetermined depth that the punch tip 326 is to be driven into the bone 670, which may allow the user (e.g., surgeon) to visualize the punch tip 326 and k-wire as they are driven into the bone 670. The k-wire 320 may then be inserted through the punch 322 which is within the channel 212 to contact and/or extend through the punch tip 326. Following such insertion, the exposed end 321 of the k-wire 320 may extend from the punch 322 in a direction opposite that which the punch 322 was inserted into the sleeve 200. The user may then use the punch handle 324 to ergonomically control the punch 322 throughout the procedure.

The k-wire cap 300 may then be placed over the exposed end 321 of the k-wire 320, such that the exposed end 321 is received in the long cavity 312 and the k-wire cap 300 contacts the punch 322, as shown in FIG. 23. The long cavity 312 may have a length which is equal to or greater than a length of the exposed k-wire 320 from the exposed end 321 to the punch 322 and may therefore permit the first impact to be applied to the punch 322 and the k-wire 320 such that both are driven into the bone to the same distance (e.g., the first predetermined depth). The first impact may thus be made to prevent driving the k-wire 320 further into the bone 670 than the punch tip 326. As noted above, the punch tip 326 may be hollow and may include a channel configured to permit the k-wire 320 to expend through and beyond the punch tip 326 and into the bone 670 to the second predetermined depth, which may be further than the first predetermined depth. The k-wire cap 300 may therefore ensure that the k-wire 320 avoids being driven further than the punch tip 326 in a direction towards the tendon 672 and/or the bone 670 in response to the first impact.

The k-wire cap 300 may utilize the k-wire 320 for lateral stability. That is, the k-wire cap 300 may be placed over the k-wire 320 to align the k-wire cap 300 with the punch 322 and/or the sleeve 200. Whenever the cap 300 is placed over the k-wire 320, it may be that the k-wire cap 300, the k-wire 320, the punch 322, and/or the sleeve 200 are aligned along the second longitudinal axis 332. This alignment may reduce the risk of components slipping or moving out of place when the first impact is applied. The alignment may also assist in directing the force of the first impact substantially in the same direction as the inserting of the punch 322 into the channel 212 of the sleeve 200. The alignment indicator 224 may assist in determining whether the components are properly aligned.

The first impact may then be applied, for example, by hammering or malleating, to the second end 304 of the k-wire cap 300. The second end 304 may have a substantially flat or planar surface to allow for solid, controllable contact when applying the first impact. The k-wire cap 300 may impact the punch 322 and/or the k-wire 320 in response to the first impact. The first impact may then drive the punch tip 326 through the tendon 352 and into the bone 350 to the first predetermined depth, such that the first gap 328 between the punch handle 324 and the sleeve 200 may be closed and the punch handle 324 may contact the sleeve 200. The punch handle 324 may thus act as a stop which may prevent the punch tip 326 from being driven into the bone 350 beyond the first predetermined depth in response to the first impact. As noted above, the region including the bone 670 and the tendon 672 may be imaged or scanned here to determine whether the punch tip 326 has been properly driven into the bone 670 to the first predetermined depth.

Once the punch tip 326 has pierced the bone 670 to the first predefined depth, the k-wire cap 300 may be reversed and/or flipped over such that the exposed end 321 may be received within the short cavity 318 of the k-wire cap 300 and the second gap 330 exists between the k-wire cap 300 and the punch 322, as shown in FIG. 24. The second gap 330 may have a length which is equal to the second predetermined depth, as described above. The second predetermined depth may be the depth that the k-wire 320 is expected to be driven into the bone 670 in response to the second impact applied to the k-wire 320. In some embodiments, specifically with respect to biceps tenodesis procedures, the second predetermined depth may be about one inch.

The second impact may then be applied, for example, by hammering or malleating, to the first end 302 of the k-wire cap 300. The first end 302 may be substantially flat or planar surface to allow for solid, controllable contact when applying the second impact. The k-wire cap 300 may impact the k-wire 320 in response to the second impact to drive the k-wire 320 through the punch tip 326 and into the bone 670 to the second predetermined depth. The second impact may allow the second gap 330 between the k-wire cap 300 and the punch 322 to be closed and the k-wire cap 300 may contact the punch 322. The punch 322 may act as a stop which may prevent the k-wire 320 from being driven into the bone 670 beyond the second predetermined depth in response to the second impact. As noted above, the region including the bone 670 and the tendon 672 may be imaged or scanned here to determine if the k-wire 320 has been properly driven into the bone 350 to the second predetermined depth.

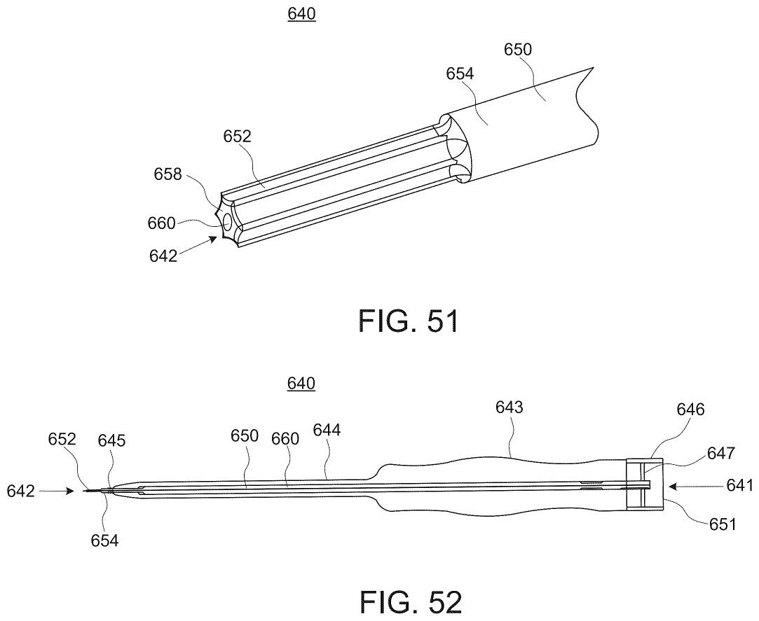

Once the k-wire 320 and the punch tip 326 have been inserted through the tendon 672 and into the bone 670, the k-wire cap 300 may be removed and the punch 322 may be back-tapped (e.g., by hammering or malleating) and removed, leaving the sleeve 200 and the k-wire 320 in place, as shown in FIGS. 25-26. Removal of the punch 322, including the punch tip 326, may leave a cavity in the bone 670 having a depth which is equal to the first predetermined depth and is configured to receive the implant 400. The implant 400 may then be prepared to be inserted, for example, by being attached to the inserter 640 as described above. Notably, however, in such an embodiment, the inserter 640 may include a channel 660 (FIGS. 51-52) to removably receive the k-wire 320, such that both the implant 400 and the inserter 640 may be placed over the k-wire 320 during insertion of the implant 400. By creating the cavity in the bone 670 according to the method disclosed herein, the user (e.g., surgeon) may avoid the need to drill the bone 670. The present method may enable a user to repair the tendon 672 (e.g., a torn and/or severed tendon) by compressing the tendon 672 wherever the surgeon prefers along the bone 670, for example, along the humeral bone during a biceps tenotomy.

The inserter 640 may be used to assist in inserting the implant 400 into the cavity left in the bone 670 by the removal of the punch tip 326, as shown in FIG. 27. More specifically, the inserter 640 may assist in inserting the implant 400 over, around and/or down the exposed end of the k-wire 320. In some embodiments, the implant 400 may receive the k-wire 320 in the cannula 430. Further, the inserter 640 may assist in inserting the implant 400 into and/or through the channel 212 of the sleeve 200 to reach the cavity in the bone 670. During this process, the threaded end 645 of the inserter may be engaged with the first inner surface 426 of the implant 400 as described above such that the implant 400 is fixed to the inserter 640 during insertion.

The channel 212 of the sleeve 200 and/or the k-wire 320 may assist in guiding the implant 400 toward and/or into the cavity in the bone 670 during insertion of the implant 400. For example, the punch tip 326 may be configured (e.g., sized, shaped and/or dimensioned) to direct the k-wire 320 through the punch tip 326 and through a center of the cavity in the bone 670 by removing the punch tip 326 following the second impact. The k-wire 320 may then be used to guide and/or center the implant 400 with respect to the cavity in the bone 670. During insertion of the implant 400 into the channel 212, the implant 400, the k-wire 320, the sleeve 200, and/or the inserter 640 may be longitudinally aligned along the first longitudinal axis 106 of the implant 400.

The implant 400 may slide or otherwise progress down the k-wire 320 to contact the tendon 672, at which point a third impact may be applied (e.g., by hammering or malleating) to the inserter 640 to drive the implant 400 through the tendon 672 and/or the bone 670 to the first predetermined depth to fully seat the implant 400. In some embodiments, the third impact may be applied to the flat end face 651 of the knob 646 of the inserter 640. The third impact may instead be multiple third impacts which progressively drive the implant 400 through the tendon 672 and/or into the bone 670. As shown in FIGS. 46 and 50, the tendon 672 may be compressed to the bone 670 by the bottom surface 414 and/or the protrusions 418 of the implant 400 while maintaining natural anatomic tensioning of the tendon 672. Once inserted, the inserter 640 may be used to transition the implant 400 from the first state 446 to the second state 448 as described above to secure the implant 400 in place in the cavity in the bone 670. The ridges 424 of the implant 400 may engage the bone 670 to further secure the implant 400 within the cavity in the bone 670 by resisting pullout. Once the implant 400 has been properly secured within the cavity in the bone 670, then the sleeve 200, the inserter 640, the k-wire 320 and/or any other components not intended to remain in the patient's body (e.g., within the cut) after the cut has been closed may be removed to complete the surgical procedure. In some embodiments, completing the surgical procedure may include stitching or otherwise closing the cut or incision.

When the implant 400 is inserted, the cannula 430 of the implant 400 may contact the intramedullary region of the bone 670 and may be in fluid communication with the intramedullary region of the bone 670. Also, when the implant 400 is inserted, the venting holes 440, 441 may further be in fluid communication with the space or region between the bone 670 and the tendon 672. The implant 400, via the cannula 430 and the venting holes 440, 441 may therefore facilitate the transport of biological agents, for example, healing agents, from the intramedullary region to the region between the bone 670 and the tendon 672, which may promote healing of the bone 670, the tendon 672, and/or surrounding tissue(s).

The implant 400 is designed to be usable in boney tissue(s) of any hardness(es). However, the density of the boney tissue into which the implant 400 is inserted may influence the expansion of the wings 432, 434. For example, the wings 432, 434 may extend further away from each other within softer boney tissue(s) during the transition from the first state 446 to the second state 448 as compared to harder bone. However, in either case, the wings 432, 434 are suitable to expand during the transition from the first state 446 to the second state 448 in order to secure the implant 400 therein.

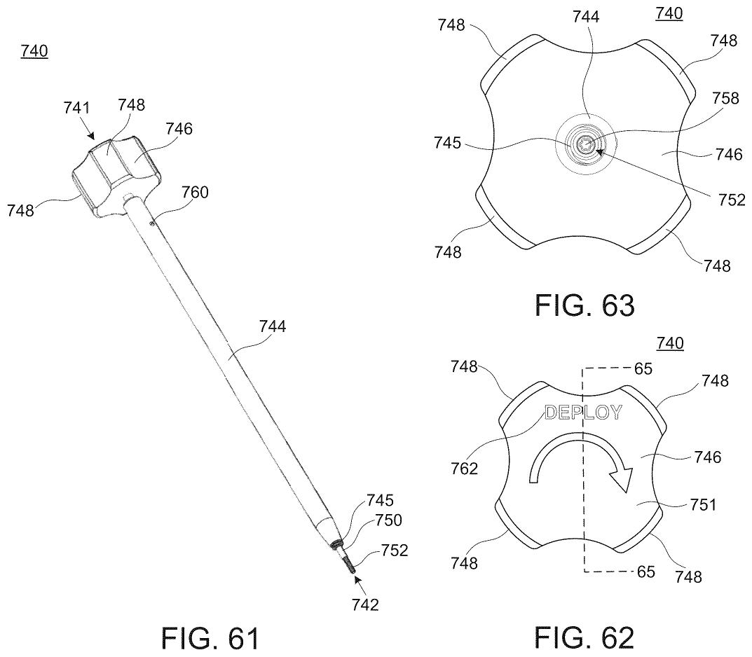

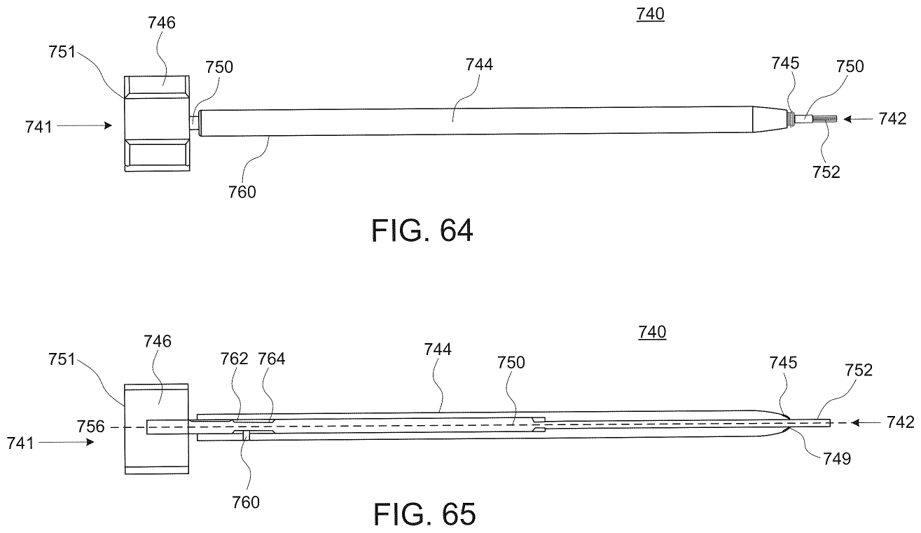

FIGS. 61-65 depict an alternative embodiment of an inserter 740 (e.g., an alternative embodiment of the inserter 640). The inserter 740 may be similar to the inserter 640 in its function and/or its use in the method as described above. The inserter 740 may extend from a first end 741 to a second end 742, and may include a shank or member 744, a knob 746, and a rod 750. Notably, the inserter 740 may lack the handle 643 that is a feature of the inserter 640 (e.g., FIG. 38), and instead the member 744 may extend along the inserter 740 between (or substantially between) the threaded end 745 and the knob 746. The member 744 may have, for example, a uniform diameter along its'length between the end positioned near the first end 741 of the inserter 740 and the tapered portion near the second end 742 of the inserter 740. The tapered portion may end at the threaded end 745 where the diameter is smaller than the greatest diameter of the member 744. In some embodiments, the knob 746 may contact the member 744 and/or may extend from or substantially from the first end 741 to the handle 743. In other embodiments, there may be a gap between the knob 746 and the member 744 through which the rod 750 may be visible.

The knob 746 may include one or a plurality of knob protrusions 748, which may be ergonomically configured (e.g., sized, shaped and/or dimensioned) to be grasped by a user (e.g., a surgeon) to twist the knob 746. Further the knob 746 may have an end face 751 located, for example, at the first end 741 of the inserter 740. The end face 751 of the knob 746 may be, for example, flat or substantially flat, or otherwise configured (e.g., sized, shaped and/or dimensioned) to receive the first impact to drive the implant 400 into the bone, as described above.

The member 744 may extend from a position near the knob 746 to an exposed portion of the rod 750 featuring a tip 652. The member 744 may include a threaded end 745, for example, on an end of the member 644 opposite the knob 746. As shown in FIG. 65, the threaded end 745 may include an opening 749 through which the rod 750 may extend. The threaded end 745 may further be configured (e.g., sized, shaped and/or dimensioned) to engage with the implant 400, for example, with the threading of the first inner surface 426, as shown in FIG. 67.

In some embodiments, the rod 750 may extend through an interior of the inserter 740, for example, longitudinally through at least a portion of the knob 746 and/or the member 744, as shown in FIG. 65. Said another way, the rod 750 may extend longitudinally through a center of the inserter 740 along a fourth longitudinal axis 756. The rod 750 may further extend through the opening 749 of the member 744 in a direction from the first end 741 towards the second end 742, such that the tip 752 of the rod 750 may exit the opening 749 so as to be exposed to an exterior of the inserter 740 for engagement with at least a portion of the implant 400. In some embodiments, the knob 746 and the rod 750 may be monolithically formed (e.g., a unitary and/or one-piece construction), or may be made from separate components that are later combined. In further embodiments, the knob 746 may be detachable from the rod 750, for example, when cleaning.

With continued reference to FIG. 65, the inserter 740 may include a pin, screw or like component 760 which is inserted into a cavity 762 located between a thin portion 764 of the rod 750 and an interior of the member 744 to fix, secure, fasten, or otherwise connect the rod 750 and the member 744. The pin 760 may also allow for limited movement of the rod 750 within the member 744, for example, along the fourth longitudinal axis 756. The pin 760 may be removed to allow for the rod 750 to be detached from and/or removed from the member 744, such as during cleaning of the inserter 740 following its use in a surgical procedure.

The tip 752 of the rod 750 may be, in some embodiments, identical to the tip 652 of the rod 650 (e.g., FIG. 43). The tip 752 may thus be configured (e.g., sized, shaped and/or dimensioned) to be inserted into the cavity 458, or another cavity of another implant, such as implant 900, as described below. In some embodiments, the tip 752 may have a polygonal end 758 of any shape and/or dimension which permits engagement of the tip 752 with a corresponding geometry of a cavity of a hard tip (e.g., the cavity 458). In an example, the tip 752 may have the shape of a standard drive and/or screwdriver head, such as a hexalobular, torx, 6-point or star screwdriver head, and the cavity 458 may be of a corresponding geometry which permits the cavity 458 to removably receive the tip 752 and/or the polygonal end 758. In other examples, the tip 752 and the polygonal end 758 may be of any other corresponding geometry to a cavity of a hard tip to permit the cavity to removably receive the tip 752 and/or the polygonal end 758. In an example, when the tip 752 is engaged with the cavity 458, the first longitudinal axis 406 and the fourth longitudinal axis 756 may be aligned (e.g., overlap).

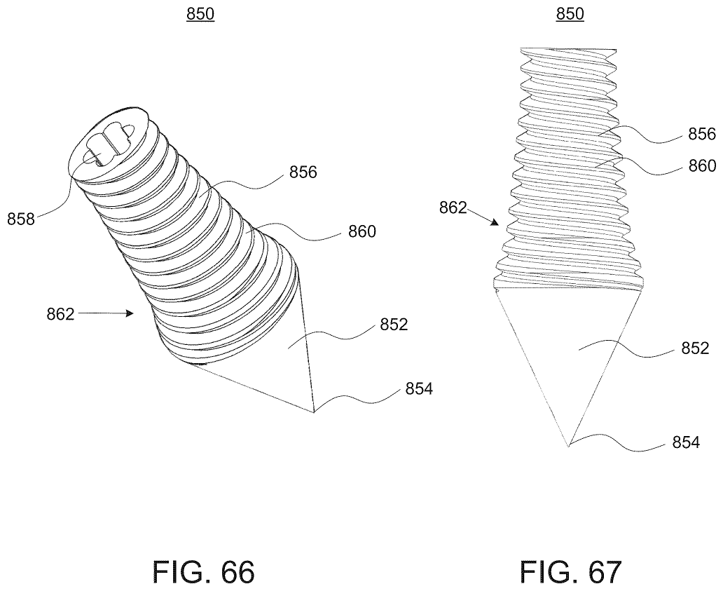

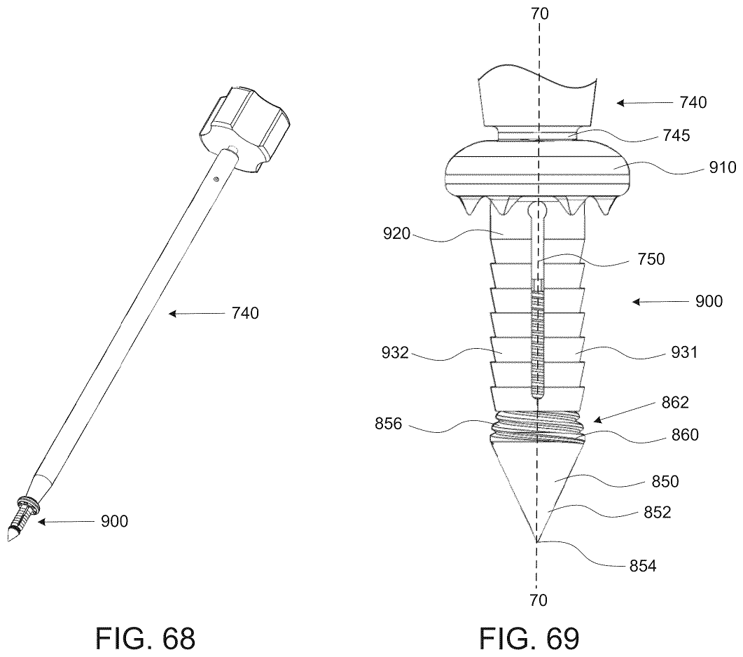

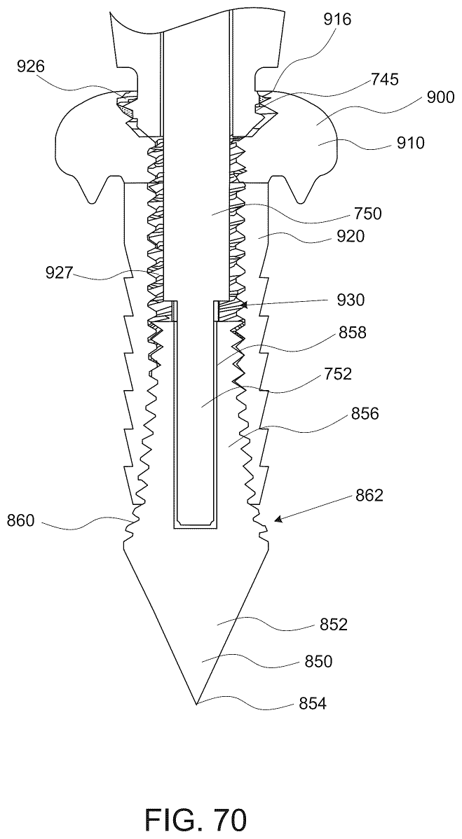

While the description thus far has focused on an embodiment of the implant 400 which includes the hard tip 450, there may be alternative embodiments of the implant and/or the hard tip which may be employed. For example, FIGS. 66-67 depicts a hard tip 850 (e.g., an alternative embodiment of the hard tip 450), and FIGS. 69-70 depict the hard tip 850 located in an implant 900 (e.g., an alternative embodiment of the implant 400) and engaged with the inserter 740. FIG. 68 shows the full inserter 740 engaged with the implant 900. The implant 900 may be identical to the implant 400 except in that a stem 920 may be shaped differently than the stem 420 to accommodate the hard tip 850. More specifically, a cannula 930 of the implant 900 may widen towards an end of the implant 900 which receives the hard tip 850, as shown in FIG. 68.

The hard tip 850 may include a head 852 connected to a stem 856. The head 852 may include a tip or point 854, for example, at an end of the head 852 opposite of the connection to the stem 856. When the implant 900 is in the first state 446, the stem 856 of the hard tip 850 may be located in the cannula 930 such that the head 852 extends out of the cannula 930 to an exterior of the implant 900 and the point 854 faces away from the cannula 930. The point 854 may be sharp and may further be configured (e.g., sized, shaped and/or dimensioned) to be driven through soft tissue (e.g., a tendon) and bone to seat the implant 900. In some embodiments, the stem 856 may further include a tapered portion 862 featuring a threading 860. In some embodiments, the hard tip 850 may include a cavity 858, which may be configured (e.g., sized, shaped and/or dimensioned) to receive the tip 752 and/or the polygonal end 758 of the inserter 640.

The stem 856 of the hard tip 850 may be configured (e.g., sized, shaped and/or dimensioned) to be received in the stem 920, for example, within the cannula 930, as shown in FIG. 67. The stem 856 may include the threading 860, which may be configured (e.g., sized, shaped and/or dimensioned) to engage with a corresponding threading of a second inner surface 927 of the stem 920 to allow the hard tip 850 to be drawn or otherwise pulled up through the cannula 930 towards the head 410 of the implant 900 in response to a twisting, turning or rotating of the hard tip 850. Such twisting, turning or rotating may work the same as with the hard tip 450 described above. Just as with the implant 400, the implant 900 may include at least a first wing 931 and a second wing 932, as shown in FIG. 69. In some embodiments, the first wing 931 and/or the second wing 932 may expand away from the hard tip 850 in response to the hard tip 850 being drawn up into the cannula 930 to secure the implant 900 within a bone.

The hard tip 850 may be pre-assembled or pre-molded into the implant 900. For example, the implant 900 (e.g., the stem 920 and/or the cannula 930) may be formed around the hard tip 850 (e.g., around the stem 856) or the hard tip 850 may be inserted into the implant 900 during formation thereof. Alternatively, the hard tip 850 may be screwed into the implant 900 such as by inserting the stem 856 into the cannula 930 such that the threading 860 engages with the corresponding threading of the second inner surface 927. The hard tip 850 may then be rotated to advance the hard tip 850 into the cannula 930 along the second inner surface 927 in a direction from the hard tip 850 towards a head 910 of the implant 900 to transition the implant 900 from the first state to the second state.

The cavity 858 of the hard tip 850 may be configured (e.g., sized, shaped and/or dimensioned) to engage with a portion of an inserter (e.g., the inserter 640 and/or the inserter 740) to transition the implant from a first state (e.g., the first state 446) to a second state (e.g., the second state 448). For example, the tip 752 of the inserter 740 may be turned while engaged with the cavity 858 to progress the hard tip 850 along the threading of the second inner surface 927 in a direction from the hard tip 850 towards the head 910. As the hard tip 850 moves upward into the cannula 930, a pressure or force may be applied to the stem 920 (e.g., to breakaway points which may be the same or similar to breakaway points 442, 443) by the tapered portion 962. The force or pressure may be sufficient to break, snap, or otherwise disconnect the breakaway points to allow for the stem 920 to separate (e.g., for the first wing 931 and the second wing 932 to expand outwardly) in the same way as described above with respect to the implant 400.

With reference to FIGS. 69-70, the tip 752 of the inserter 740 may access the cavity 858 of the hard tip 850 by being inserted into the implant 900 through an opening 916 in the head 910 and through the cannula 930. The threaded end 745 of the inserter 740 may be configured (e.g., sized, shaped and/or dimensioned) to engage with a corresponding threading of the first inner surface 926 of the implant 900 to secure the inserter 740 (and thus the tip 752) with respect to the implant 900. The tip 752 may be locked into, connected to, or otherwise secured within the cavity 858 via engagement of the threaded end 745 with the threading of the first inner surface 926. For example, when the tip 752 is inserted into the cavity 858, the member 744 including the threaded end 745 may be rotated to secure the threaded end 845 to the first inner surface 926. In some embodiments, the member 744 may be rotated to secure (e.g., removably secure) the threaded end 845 to the first inner surface 926 without also rotating the rod 750 and/or the knob 746.

The threaded end 845 may be unlocked, disconnected, or otherwise unsecured from the first inner surface 926 by a rotation of the member 744 in a reverse direction from that which it was initially rotated to engage the first inner surface 926. In some embodiments, the tip 752 may further be unlocked, disconnected, or otherwise unsecured from the cavity 858 following disengagement of the threaded end 745 from the first inner surface 926 as just described by moving the inserter 740 in a reverse direction from that in which the tip 752 was initially inserted into the cavity 848.

In some embodiments, the inserter 740 may be configured (e.g., sized, shaped and/or dimensioned) such that the knob 746 and the rod 750 may advantageously spin, twist, turn, and/or rotate independently from the member 744. For example, when the tip 752 is inserted into the cavity 858, the member 744 may be rotated independently of the knob 746 and/or the rod 750 to secure the threaded end 745 to the first inner surface 926. Then, the knob 746 and the rod 750 may be rotated independently of the member 744 to draw the hard tip 850 into the cannula 930 so as to transition the implant 900 from the first state to the second state.

In some embodiments, the inserter 740 may be configured (e.g., sized, shaped and/or dimensioned) such that the knob 746 and the rod 750 may advantageously spin, twist, turn and/or rotate in an opposite direction from the member 744 as described above. For example, the direction in which the member 744 is rotated to secure the threaded end 745 to the first inner surface 926 may be opposite from the direction in which the knob 746 and the rod 750 are rotated to transition the implant 900 from the first state to the second state (e.g., to draw the hard tip 850 into the cannula 930). Thus, in some embodiments, the threading of the first inner surface 926 may be different from the threading of the second inner surface 927 to facilitate this opposing rotation. The independent and/or opposing rotation(s) of the knob 746 and the rod 750 as compared to the member 744 may minimize the risks of mistakes during performance of the method, just as the independent rotations of the knob 646 and the rod 650 as compared to the handle 643 and the member 644 may minimize the risks of mistakes as described above.

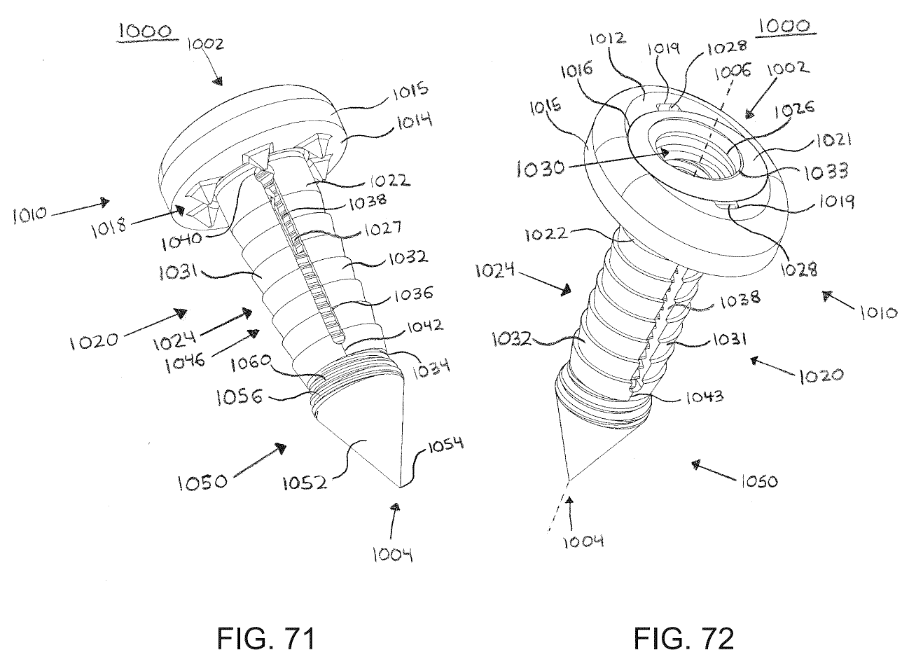

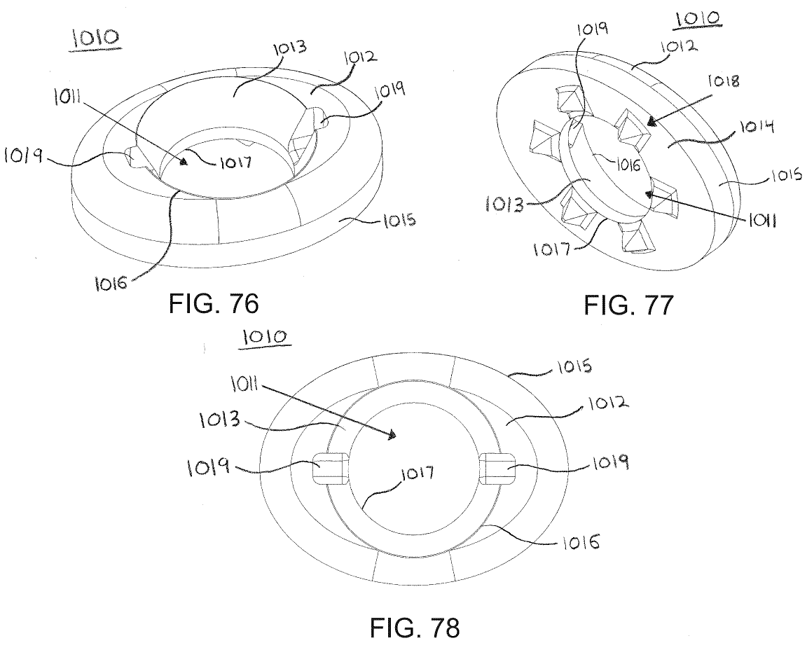

Referring now to FIGS. 71-78, another embodiment of an implant for soft tissue fixation (e.g., another embodiment of the implant 100, the implant 400, and/or the implant 900) will now be described in detail. As discussed above, an implant 1000 according to aspects described herein may include a modular head 1010 and a modular stem 1020 to allow for various differently shaped components (e.g., differently shaped heads and/or stems) to be combined and/or mixed-and-matched, as desired by a user. The implant 1000 may include the same, similar and/or overlapping features with other embodiments (e.g., the implant 100, the implant 400, and/or the implant 900) as described above.

As shown in FIGS. 71-73, the implant 1000 may have a first end 1002 and a second end 1004. The implant 1000 may further include a modular head 1010 removably and/or detachably connected to a modular stem 1020. The modular stem 1020 may include a hard tip 1050 connected to the stem 1020 at an opposite end of the stem 1020 from the connection of the stem 1020 to the head 1010. The head 1010, the stem 1020 and the hard tip 1050 may extend from the first end 1002 to the second end 1004 when the implant 1000 is assembled, and may further be aligned along a longitudinal axis 1006 of the implant 1000. When the implant 1000 is assembled, the head 1010 and/or the stem 1020 may contact the first end 1002, and the tip 1050 may contact the second end 1004, as best shown in FIGS. 71-72.

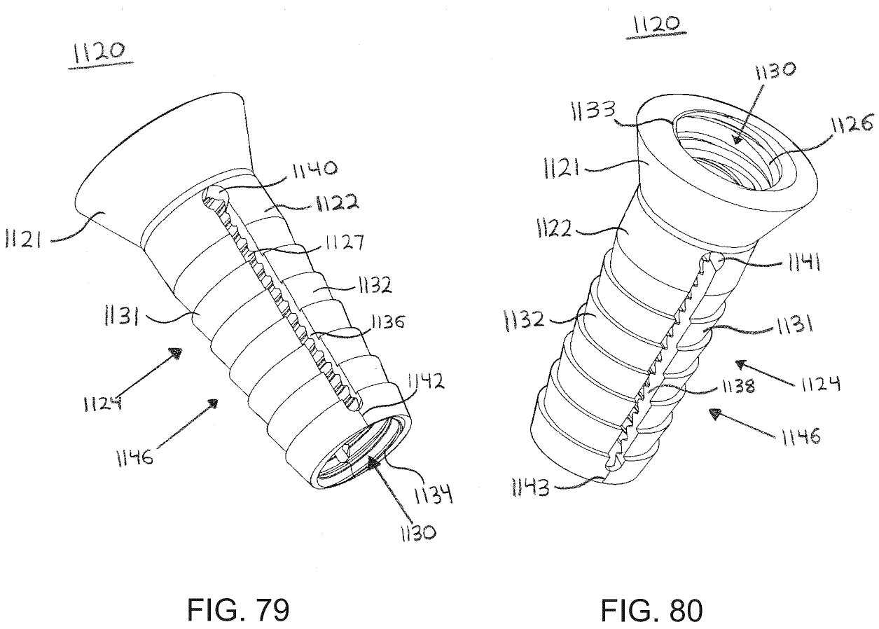

In the embodiment shown in FIGS. 71-78, the implant 1000 may be formed of a plurality of components that may be rigidly and/or detachably connected. For example, the stem 1020 (with or without the hard tip 1050) may be configured (e.g., sized, shaped and/or dimensioned) to be inserted through a hole 1011 (e.g., a through-hole) of the head 1010 to connect the head 1010 to the stem 1020. In an embodiment, the stem 1020 may be inserted through the hole 1011 in a direction from a first opening 1016 of the hole 1011 towards a second opening 1017 of the hole 1011, such that a collar 1021 of the stem 1020 rests flush against an inner surface 1013 of the hole 1011. Similarly, the stem 1020 may be removed through the hole 1011 in an opposite direction, e.g., a direction from the second opening 1017 of the hole 1011 towards the first opening 1016 of the hole 1011. For example, the stem 1020 may be removed from the hole 1011 of the head 1010 to allow for the stem 1020 to be used with a head of a different shape, as explained below in more detail with respect to at least FIGS. 79-92.

Continuing with the embodiment shown in FIGS. 71-78, the collar 1021 of the stem 1020 may include one or more anti-rotation protrusions 1028 configured (e.g., sized, shaped and/or dimensioned) to be inserted into and/or received by one or more corresponding anti-rotation cavities 1019 of the head 1010 to secure and/or fix the stem 1020 relative to the head 1010 such that rotation of the stem 1020 relative to the head 1010 is prevented. Each protrusion of the anti-rotation protrusions 1028 may engage with a corresponding anti-rotation cavity of the anti-rotation cavities 1019. Thus, the anti-rotation protrusions 1028 may be spaced and/or oriented about the collar 1021 to the same degree as the anti-rotation cavities 1019 are spaced, positioned and/or oriented about the inner surface 1013 of the head 1010 to ensure those components may engage as described. When the anti-rotation protrusions 1028 are engaged with the anti-rotation cavities 1019, the head 1010 and the stem 1020 may be rotatably fixed with respect to each other. This may be advantageous, for example, during insertion of the stem 1020 into the bone and/or during a drawing up of the tip 1050 into the stem 1020 when the implant 1000 is being transitioned from a resting state 1046 (e.g., similar or identical to the resting state 446) to an active state (e.g., similarly to or identical to the active state 448), requiring rotational force(s) to be exerted on the implant 1000 (e.g., using an inserter, as described above).

In the embodiment shown in FIGS. 71-78, there are two anti-rotation protrusions 1028 corresponding to two anti-rotation cavities 1019. However, in other embodiments (not shown), there may be more or less anti-rotation protrusions 1028 and anti-rotation cavities 1019.

In an embodiment shown in FIGS. 71-73 and 76-78, the head 1020 may be substantially ovular and may further include a top surface 1012 opposite to a bottom surface 1014. The top surface 1012 may be connected to the bottom surface 1014 by a side surface 1015 and/or the inner surface 1013. In an embodiment, the head 1010 may include the hole 1011, which may be formed of the first opening 1016, the second opening 1017 and the inner surface 1013. The first opening 1016 may be located on and/or in the top surface 1012, the second opening 1017 may be located on and/or in the bottom surface 1014, and the inner surface 1013 may extend from the first opening 1016 to the second opening 1017.

In an embodiment, the inner surface 1013 may narrow from the first opening 1016 to the second opening 1017 to receive the collar 1021 as described above. The collar 1021 may be wider than at least the second opening 1017 to ensure the stem 1021 cannot pass through the hole 1011 during insertion of the stem 1020 into the head 1010 (e.g., during a surgical procedure). In the embodiment shown, the collar 1021 and the inner surface 1013 have corresponding geometries, such that an outer surface of the collar 1021 may contact, abut and/or otherwise rest (e.g., flush) against the inner surface 1013 when the stem 1020 is fully inserted into and/or through the hole 1011. Similarly, in the embodiment shown, the anti-rotation protrusions 1028 and the anti-rotation cavities 1019 have corresponding geometries, such that when the outer surface of the collar 1021 rests against the inner surface 1013, the anti-rotation protrusions 1028 are located within the anti-rotation cavities 1019 to restrict and/or prevent movement (e.g., rotational movement) of the stem 1020 within the hole 1011.

The bottom surface 1014 of the head 1010 may be configured (e.g., sized, shaped, and/or dimensioned) to compress a soft tissue (e.g., a tendon) to a bone when the implant 1000 is inserted into a bone, such that the tendon may be fixed in place relative to the bone. Thus, in some embodiments, the bottom surface 1014 of the head 1010 may include a gripping means, for example, a plurality of spikes or protrusions 1018. The protrusions 1018 may be structured and/or may function identically to or substantially similarly to the protrusions 418, and thus their structure and function is not described again here in detail for the sake of brevity.

With reference to FIGS. 71-75, the stem 1020 may include an exterior or outer surface 1022, and a cannula 1030 of the implant 1000 may be located in and/or extend through all or a portion of the stem 1020 (e.g., along the longitudinal axis 1006). In an embodiment, the cannula 1030 may extend through the entire length of the stem 1020, e.g., in a direction from the first end 1002 towards the second end 1004 along the longitudinal axis 1006. Thus, when the implant 1000 is assembled, the cannula 1030 may extend through both the head 1010 and the stem 1020, as shown in FIGS. 71-72.

The cannula 1030 may include a first opening 1033, a first inner surface 1026, a second inner surface 1027, and/or a second opening 1034. In an embodiment, the first opening 1033 may be located in and/or on the collar 1021 of the stem 1020 and/or at the first end 1002 of the implant 1000 (e.g., when the implant 1000 is assembled). In an embodiment, the second opening 1034 may be located on an opposite end of the stem 1020 from the first opening 1033. In an embodiment, the first inner surface 1026 may extend between the first opening 1033 and the second inner surface 1027, and the second inner surface 1027 may extend between the first inner surface 1026 and the second opening 1034.

In some embodiments, one or both of the first inner surface 1026 and/or the second inner surface 1027 may include threads which may be configured (e.g., sized, shaped and/or dimensioned) to engage with a tool, such as an inserter (e.g., the inserter 340, the inserter 640, and/or the inserter 940) and or the hard tip 1050. The threads of the first inner surface 1026 may be the same or different from the threads of the second inner surface 1027.

In some embodiments, the stem 1020 may also include a plurality of teeth or ridges 1024 positioned along the outer surface 1022 of the stem 1020. The plurality of ridges 1024 may be structured and/or may function similarly to or identically to the plurality of ridges 124 and/or the plurality of ridges 424, and thus the structure and function of the ridges 1024 are not described here in detail for brevity's sake.

In the embodiment shown in FIGS. 71-75, the stem 1020 may include several features that are similar or identical to corresponding structures of previously described embodiments. For example, the stem 1020 may include a first side slit 1036 and/or a second side slit 1038 that may be identical to or similar to the first side slit 436 and the second side slit 438, respectively, in both structure and function. Similarly, the first side slit 1036 and the second side slit 1038 may divide at least a portion of the stem 1020 into a first expandable wing 1031 and a second expandable wing 1032, just as the first side slit 436 and the second side slit 438 may divide at least a portion of the stem 420 into the first expandable wing 431 and the second expandable wing 432.

Further, the first expandable wing 1031 and a second expandable wing 1032 of the implant 1000 may be expanded away from each other in response to a drawing up of the hard tip 1050 into the cannula 1030, similarly or identically to how the first expandable wing 431 and the second expandable wing 432 of the implant 400 may be expanded away from each other in response to the drawing up of the hard tip 450 into the cannula 430 as described above. The first side slit 1036 may thus feature a first venting hole 1040 and/or a first breakaway point 1042, and the second side slit 1038 may feature a second venting hole 1041 and/or a second breakaway point 1043. The venting holes 1040, 1041 of the implant 1000 may enable the delivery of biological healing agents and/or act as stress reducers, similarly to or identically to the venting holes 440, 441 of the implant 400, as also described above. The breakaway points 1042, 1043 of the implant 1000 may thus enable the wings 1031, 1032 to be separated and expanded away from each other to transition the stem 1020 from the resting state 1046 to an active state when the stem 1020 (e.g., as part of the implant 1000) is inserted into and/or otherwise seated within a bone. This separation and/or expansion may be identical to or similar to how the breakaway points 442, 443 may enable the wings 431, 432 of the implant 400 to be separated and/or expanded away from each other during a transition from the resting state 446 to the active state 448, as described above.

With reference to FIGS. 71-73, the hard tip 1050 of the implant 1000 may be similar or identical to the hard tip 450 of the implant 400 or the hard tip 850 of the implant 800. In the embodiment shown, the hard tip 1050 features a head 1052 (e.g., similar or identical to the head 852), a point or tip 1054 (e.g., similar or identical to the tip 854), a stem 1056 having a tapered portion 1062 (e.g., similar or identical to the stem 856 having the tapered portion 862), a cavity 1058 (e.g., similar or identical to the cavity 858). The structure and function of the hard tip 1050 is thus not described here in detail for brevity's sake.

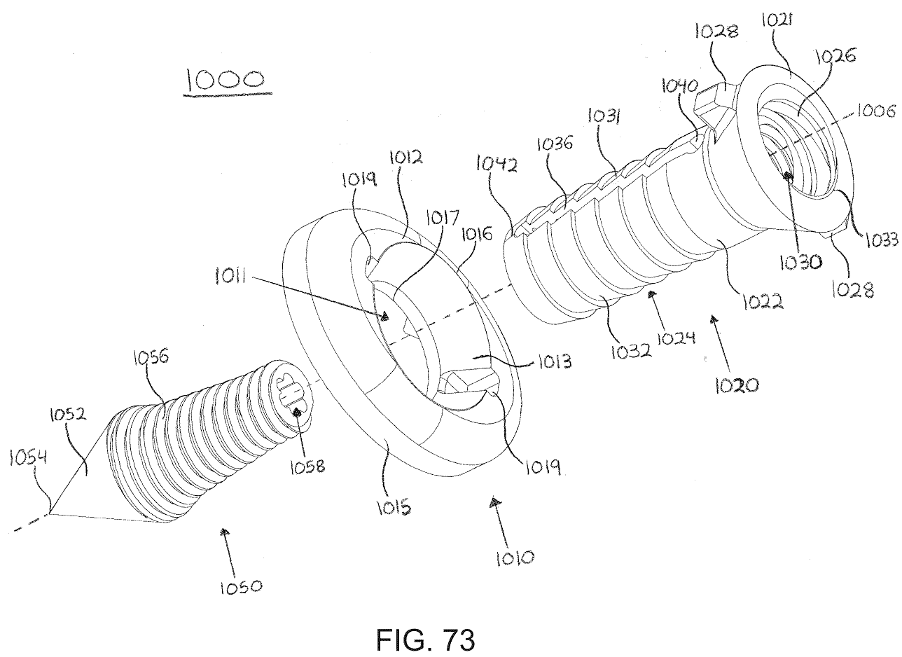

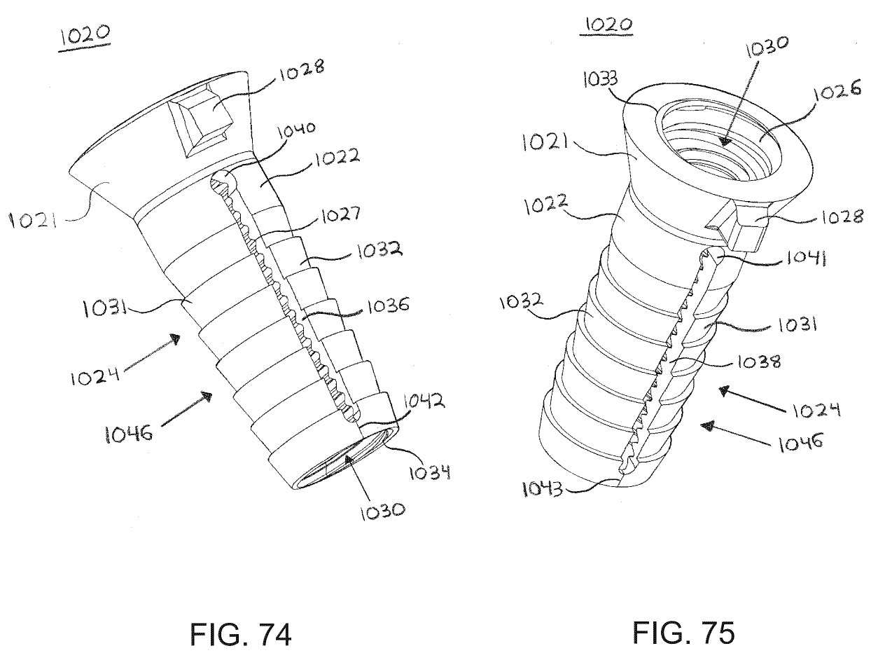

In some embodiments, the implant 1000 may lack the anti-rotation features (e.g., the anti-rotation cavities 1019 and/or the anti-rotation protrusions 1028). For example, FIGS. 74-75 show a modular stem 1120 in a resting state 1146 which is similar or identical the resting state 1046 of the stem 1020. However, the stem 1120 may include a collar 1121 that lacks the anti-rotation protrusions 1028 found on the stem 1020. Besides this difference, the stem 1120 may include several similar or identical components to the stem 1020, such as: an outer surface 1122 (e.g., similar or identical to the outer surface 1022), a cannula 1130 (e.g., similar or identical to the cannula 1030) including a first opening 1133 (e.g., similar or identical to the first opening 1033), a first inner surface 1126 (e.g., similar or identical to the first inner surface 1026), a second inner surface 1127 (e.g., similar or identical to the second inner surface 1027) and a second opening 1134 (e.g., similar or identical to the second opening 1034), a plurality of ridges 1124 (e.g., similar or identical to the plurality of ridges 1024), a first side slit 1136 (e.g., similar or identical to the first side slit 1036) having a first venting hole 1140 (e.g., similar or identical to the first venting hole 1040) and a first breakaway point 1142 (e.g., similar or identical to the first breakaway point 1042), a second side slit 1138 (e.g., similar or identical to the second side slit 1038) having a second venting hole 1141 (e.g., similar or identical to the second venting hole 1041) and a second breakaway point 1143 (e.g., similar or identical to the second breakaway point 1043), a first expandable wing 1131 (e.g., similar or identical to the first expandable wing 1031), and/or a second expandable wing 1132 (e.g., similar or identical to the second expandable wing 1032).

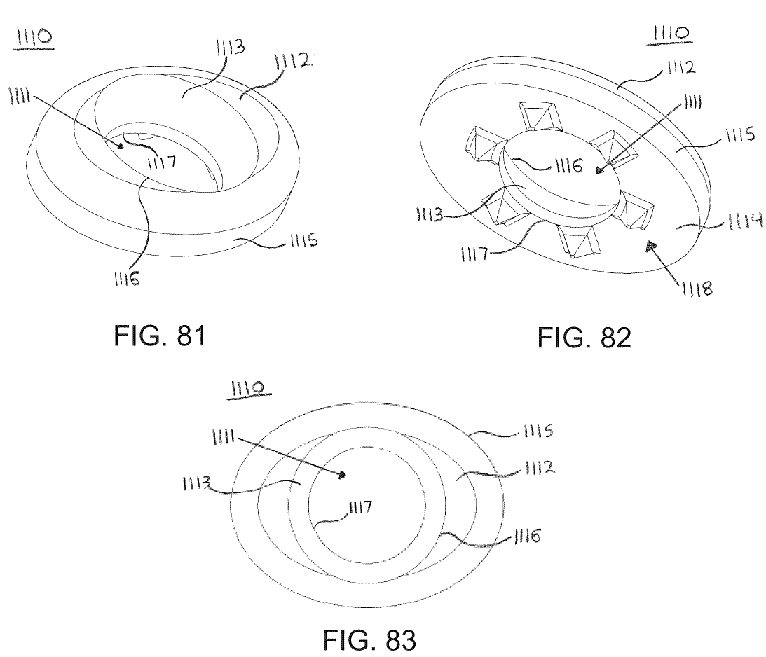

The stem 1120 may be configured (e.g., sized, shaped and/or dimensioned) to be inserted into and/or through a modular head 1110 of the kind shown in FIGS. 81-83. The head 1110 may be identical to the head 1010 of the implant 1000 except in that the head 1110 may lack the anti-rotation cavities 1019 that are features of the head 1010. Specifically, the head 1110 is shown with an ovular shape, and further includes a top surface 1112 and a bottom surface 1114 connected by a side surface 1115 and/or an inner surface 1113. The inner surface 1113 may be configured (e.g., sized, shaped and/or dimensioned) to receive the collar 1121 when the stem 1120 is inserted into and/or through a hole 1111 of the head 1110, such that an outer surface of the collar 1121 contacts, abuts, and/or otherwise rests against the inner surface 1113. The inner surface 1113 may narrow from a first opening 1116 in the top surface 1112 to a second opening 1117 in the bottom surface 1114 to ensure the stem 1120 cannot pass fully through the hole 1111 when the stem 1120 is inserted into a bone. The head 1110 may include one or more spikes and/or protrusions 1118 configured (e.g., sized, shaped and/or dimensioned) to pin a tendon and/or other soft tissue to bone (e.g., as part of a tenodesis procedure).

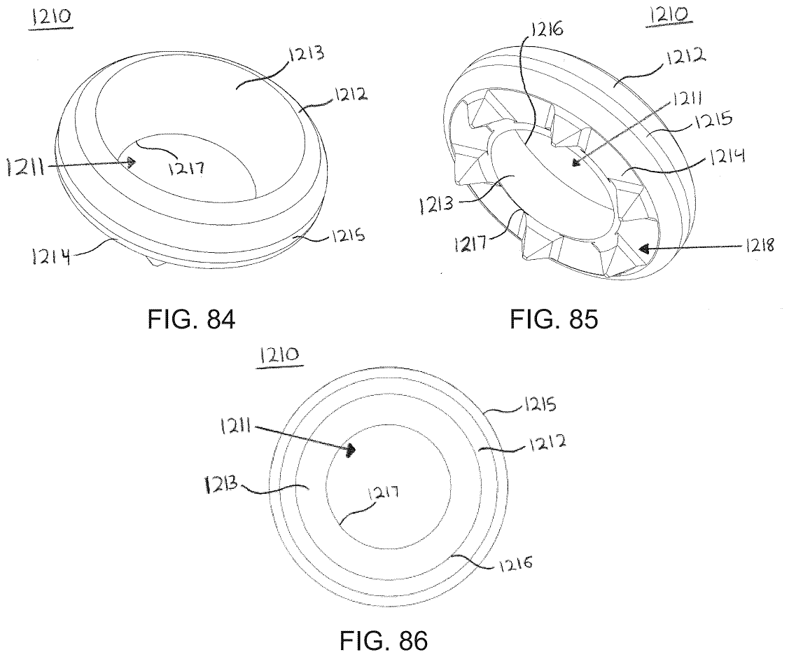

In some modular embodiments, the stem 1020 and/or the stem 1120 may be configured (e.g., sized, shaped and/or dimensioned) for use with modular heads of other shapes (e.g., shapes other than ovular as with the head 1010 and the head 1110). For example, the stem 1120 (e.g., which lacks anti-rotation protrusions) may be configured (e.g., sized, shaped and/or dimensioned) for use with a circular head 1210 as shown in FIGS. 84-86, among other shapes. In an embodiment, the head 1210 may include a top surface 1212 and a bottom surface 1214 connected by a side surface 1215 and/or an inner surface 1213. The inner surface 1213 may be configured (e.g., sized, shaped and/or dimensioned) to receive the collar 1121 when the stem 1120 is inserted into and/or through a hole 1211 of the head 1210, such that an outer surface of the collar 1121 rests against the inner surface 1213. The inner surface 1213 may narrow from a first opening 1216 in the top surface 1212 to a second opening 1217 in the bottom surface 1214 to ensure the stem 1120 cannot pass fully through the hole 1211 when the stem 1120 is inserted into a bone. The head 1210 may include one or more spikes and/or protrusions 1218 configured (e.g., sized, shaped and/or dimensioned) to pin a tendon and/or other soft tissue to bone (e.g., as part of a tenodesis procedure).

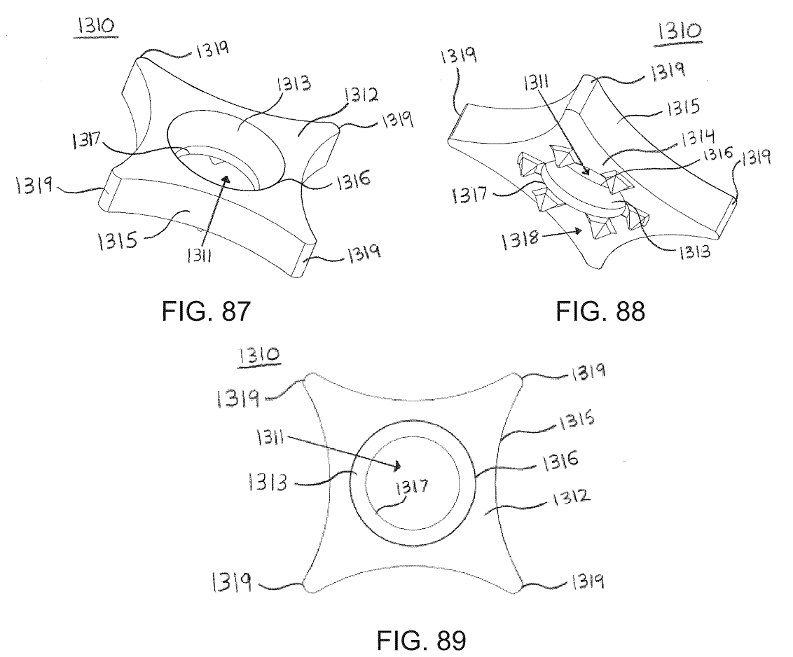

As another example, the stem 1120 may be configured (e.g., sized, shaped and/or dimensioned) for use with a substantially quadrilateral head 1310 as shown in FIGS. 87-89. In an embodiment, the head 1310 may include a top surface 1312 and a bottom surface 1314 connected by a side surface 1315 and/or an inner surface 1313. The inner surface 1313 may be configured (e.g., sized, shaped and/or dimensioned) to receive the collar 1121 when the stem 1120 is inserted into and/or through a hole 1311 of the head 1310, such that an outer surface of the collar 1121 rests against the inner surface 1313. The inner surface 1313 may narrow from a first opening 1316 in the top surface 1312 to a second opening 1317 in the bottom surface 1314 to ensure the stem 1120 cannot pass fully through the hole 1311 when the stem 1120 is inserted into a bone. The head 1310 may include one or more spikes and/or protrusions 1318 configured (e.g., sized, shaped and/or dimensioned) to pin a tendon and/or other soft tissue to bone (e.g., as part of a tenodesis procedure). The side surface 1315 may include four sides connected at various points 1319. In the embodiment shown, the sides of the head 1310 may be curved and equally long. However, in other embodiments the sides may be straight and/or may have different lengths relative to each other, depending on the use case, among other things.

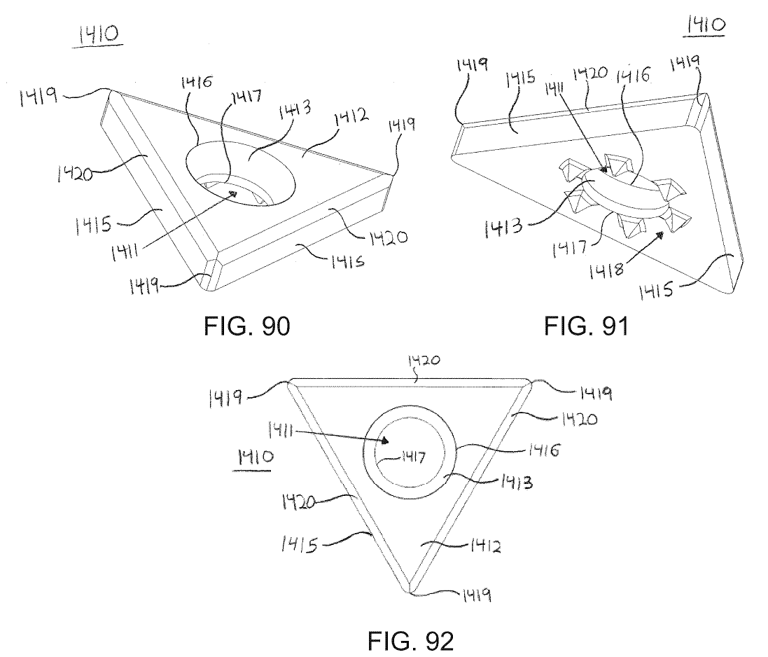

As yet another example, the stem 1120 may be configured (e.g., sized, shaped and/or dimensioned) for use with a triangular head 1410 as shown in FIGS. 90-92. In an embodiment, the head 1410 may include a top surface 1412 and a bottom surface 1414 connected by a side surface 1415 and/or an inner surface 1413. The inner surface 1413 may be configured (e.g., sized, shaped and/or dimensioned) to receive the collar 1121 when the stem 1120 is inserted into and/or through a hole 1411 of the head 1410, such that an outer surface of the collar 1121 rests against the inner surface 1413. The inner surface 1413 may narrow from a first opening 1416 in the top surface 1413 to a second opening 1417 in the bottom surface 1414 to ensure the stem 1120 cannot pass fully through the hole 1411 when the stem 1120 is inserted into a bone. The head 1410 may include one or more spikes and/or protrusions 1418 configured (e.g., sized, shaped and/or dimensioned) to pin a tendon and/or other soft tissue to bone (e.g., as part of a tenodesis procedure). The side surface 1415 may include three sides connected at various points 1419. The sides of the head 1410 may be curved or straight, depending on the use case, among other things. In the embodiment shown, the top surface 1412 may include a plurality of bevels 1420 connecting the top surface 1412 to the side surface 1415.

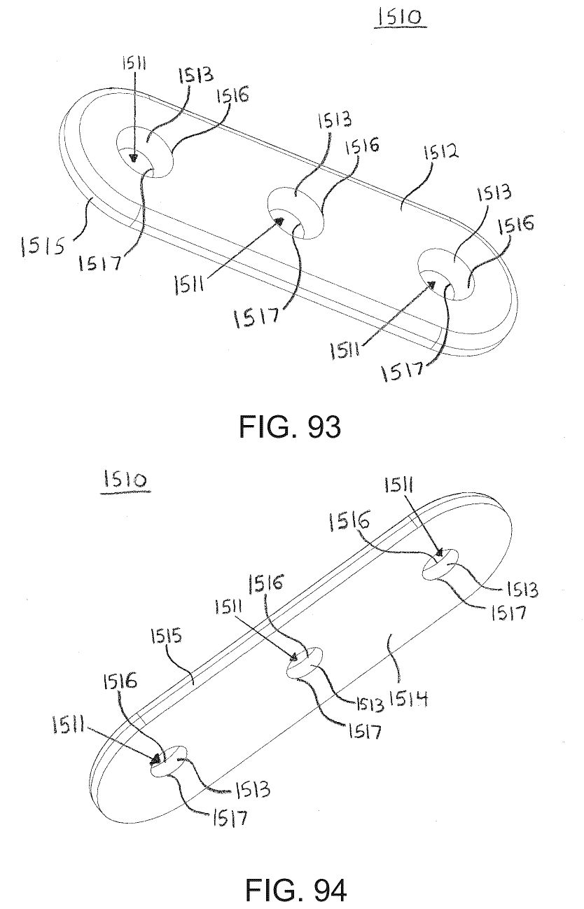

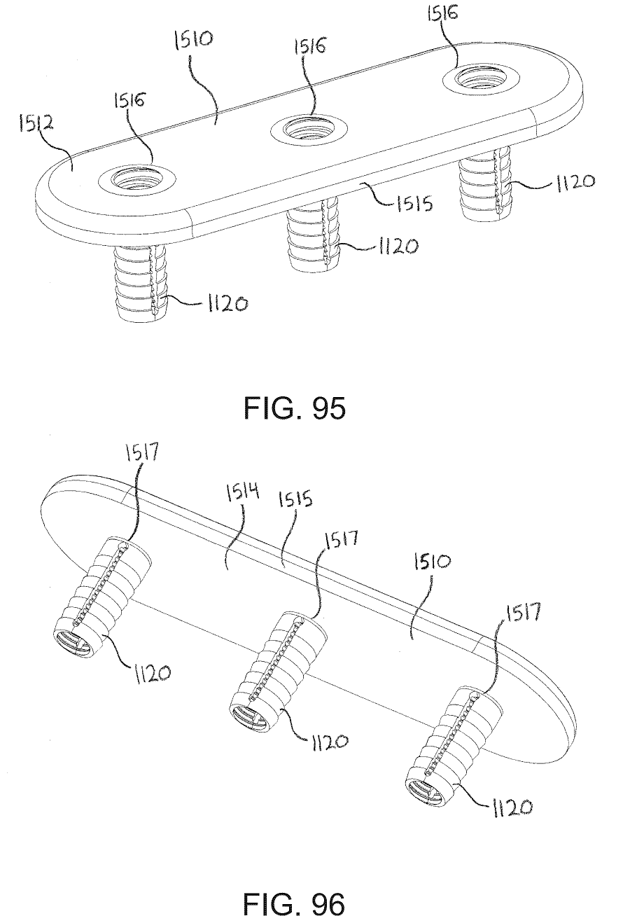

In some embodiments, one or more of the modular stem 1120 (or, alternatively, one or more of an implant including the modular stem 1120) may be used with a plate 1510 instead of a modular head (e.g., the head 1110, the head 1210, the head 1310, and/or the head 1410), as shown in FIGS. 93-96. In an embodiment, the plate 1510 may include a top surface 1512 and a bottom surface 1514 connected by a side surface 1515 and/or one or more inner surface(s) of one or more hole(s) of the plate 1510. Each hole 1511 of the holes of the plate 1510 may include an inner surface 1513 that may be configured (e.g., sized, shaped and/or dimensioned) to receive a collar 1121 of a stem 1120 when the stem 1120 is inserted through hole 1511 of the plate 1510, such an outer surface of the collar 1121 rest against the inner surface 1513. For example, each inner surface 1513 may have a shape corresponding to the geometry of the collar 1121. Each inner surface 1513 may therefore narrow from a first opening 1516 in the top surface 1512 to a second opening 1517 in the bottom surface 1514 to ensure the stem 1120 (e.g., the collar 1121 of the stem 1120) cannot pass fully through a respective hole 1511 when the stem 1120 is inserted therethrough.

The plate 1510 may advantageously be used with one or more stems 1120 (or, alternatively, with one or more of an implant including the stem 1120) to treat conditions such as bone fractures. For example, where the plate 1510 is used to treat a bone fracture, the plate 1510 may span across a fractured portion of a bone to fix the bone in place on opposite sides thereof. Thus, where multiple of the stem 1120 are used, at least a first stem 1120 may be inserted, seated and/or otherwise anchored into a first portion of a bone, and at least a second stem 1120 may be inserted, seated and/or otherwise anchored into a second portion of the bone that is opposite the first portion about the fracture to secure and/or fix the first portion of the bone and the second portion of the bone with respect to each other. In some embodiments, the plate 1510 may thus be used to secure various rigid or substantially rigid tissue(s) relative to each other, as needed.

In an embodiment not shown, the plate 1510 may include one or more spike(s) and/or protrusions configured (e.g., sized, shaped, and/or dimensioned) to pin a tendon and/or soft tissue to a bone (e.g., as part of a tenodesis procedure). Thus, in embodiments of the plate 1510 which are used to treat conditions involving soft tissue (e.g., biceps tenodesis procedure, lateral extraarticular tenodesis procedure, etc.), spikes and/or protrusions may be employed, e.g., on the bottom surface 1514, to enable the plate 1510 to be used as a means of securing and/or fixing a tendon with respect to a bone, in addition to allowing for rigid or substantially rigid structures to be fixed with respect to each other using the plate 1510 as described above. In some embodiments, the spikes and/or protrusions may be similar or identical to the protrusions 418, the protrusions 1018, the protrusions 1118, the protrusions 1218, the protrusions 1318, the protrusions 1418, and/or the protrusions 1518.

It should be noted that anti-rotation features (e.g., anti-rotation protrusions and/or anti-rotation cavities) similar or identical to those featured on the implant 1000 may be included on any of the modular stem 1120 and/or the modular heads 1110, 1210, 1310, 1410, 1510 to prevent rotation of the stem 1120 relative to any of such heads during insertion and/or seating of the stem 1120 (e.g., as assembled into an implant) into a bone. Additionally, any of the heads previously described, including the heads 110, 410 which are integrally formed to their respective stems 120, 420 may be modified to have similar or identical shapes to the shapes of any of the modular heads 1110, 1210, 1310, 1410, 1510 depending on various factors, such as patient-specific idiosyncrasies or considerations, condition-specific considerations, and/or surgical preferences, among other factors. Modular embodiments may be particularly advantageous as modularity of an implant according to aspects described herein may enable a user to swap and/or otherwise mix-and-match implant heads as-needed. In even further embodiments, alternative shapes than those described herein may be used as a head (e.g., a modular head or an integral head), again depending on various factors and/or surgical considerations.

As may be recognized by those of ordinary skill in the art based on the teachings herein, numerous changes and modifications may be made to the above-described and other embodiments of the present disclosure without departing from the scope of the disclosure. The components of the implants as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the implants may include more or fewer components or features than the embodiments as described and illustrated herein. For example, the components and features of FIGS. 1-70 may be used interchangeably and in alternative combinations as would be modified or altered by one of skill in the art. As a specific example, implants 100, 400, and 900 may be used in alternative combinations as would be modified or altered by one of skill in the art. As an additional example, the components and features of the inserters 340, 640, and 740 may be used in alternative combinations as would be modified or altered by one of skill in the art. As an even further example, the components and features of hard tips 450 and 850 may be used in alternative combinations as would be modified or altered by one of skill in the art. Accordingly, this detailed description of the currently-preferred embodiments is to be taken illustratively, as opposed to limiting of the disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. A system for compressing a tendon to a bone comprising:
a sleeve having a first end, a second end opposite the first end, and a through-hole extending longitudinally through the sleeve from the first end to the second end;
an implant configured to be inserted into the through-hole, the implant having a first engagement portion; and
an inserter configured to engage with the first engagement portion to connect the inserter to the first engagement portion, the inserter configured to be removably received within the through-hole.

2. The system of claim 1, wherein the implant further comprises:
a hard tip configured to be driven into a bone in response to at least a first impact applied to the implant.

3. The system of claim 2, wherein the inserter further comprises:
a flat end, the flat end configured to receive the at least a first impact while the inserter is engaged with the first engagement portion.

4. The system of claim 1, wherein the implant further comprises:
a hard tip translatable through a cannula of the implant to transition the implant from a first orientation to a second orientation.

5. The system of claim 4, wherein the hard tip includes a cavity, wherein the cavity receives a first portion of the inserter, wherein the first portion of the inserter is rotatable within the cavity to transition the implant from the first orientation to the second orientation.

6. The system of claim 5, wherein a body of the inserter engages with the first engagement portion and is rotatable in a first direction to secure the inserter to the first engagement portion, and wherein the first portion of the inserter is rotatable in a second direction to transition the implant from the first orientation to the second orientation, the second direction being opposite the first direction.

7. The system of claim 1, further comprising a sleeve plug removably received within the through-hole, wherein removing the sleeve plug exposes the through-hole.

8. The system of claim 1, wherein the implant further comprises:
a suture passage for receiving one or more sutures.

9. A system for compressing a tendon to a bone comprising:
an implant configured to engage with an inserter to transition the implant from a first orientation to a second orientation, the implant comprising:
a head;
a stem extending from the head;
a first cavity having a first opening in the head, the first cavity extending from the first opening through the head and into at least a portion of the stem; and
a hard tip connected to the stem.

10. The system of claim 9, wherein the stem further comprises:
at least one portal hole extending from an outer surface of the stem into the first cavity, wherein the at least one portal hole is in fluid communication with at least one opening of the first cavity.

11. The system of claim 9, wherein the stem comprises:
a plurality of slits extending longitudinally along at least a portion of the stem, the plurality of slits including at least a first slit and a second slit.

12. The system of claim 11, wherein the first slit and the second slit substantially divide the stem into a first wing and a second wing, and wherein the stem further includes at least one breakaway point, the first wing being connected to the second wing by the at least one breakaway point.

13. The system of claim 12, wherein a portion of the inserter is received in a second cavity of the hard tip, and wherein the portion is rotatable within the second cavity to draw the hard tip into the first cavity.

14. The system of claim 13, wherein the at least one breakaway point disconnects the first wing and the second wing from each other in response to the hard tip being drawn into the first cavity.

15. An implant comprising:
a head;
a stem extending from the head;
a cavity extending through the head and at least a portion of the stem; and
a hard tip connected to the stem.

16. The implant of claim 15, wherein the stem further includes at least one hole extending from an outer surface of the stem into the cavity.

17. The implant of claim 15, wherein the cavity extends through the stem from a first end of the stem to a second end of the stem.

18. The implant of claim 15, wherein the stem further comprises:
a first end contacting the head;
a second end opposite the first end; and
an outer surface;
wherein the outer surface includes a first portion and a second portion;
the first portion having a smooth surface and contacting the head; and
the second portion having one or more ridges.

19. The implant of claim 16, wherein the at least one hole further comprises a plurality of slits extending longitudinally along at least a portion of the stem, the plurality of slits including a first slit and a second slit, the first slit and the second slit substantially dividing the stem into a first wing and a second wing, and wherein the first wing and the second wing are able to be disconnected from each other at a breakaway point in response to a drawing up of the hard tip into the cavity.

20. The implant of claim 17, wherein the head is connected to the first end of the stem and the hard tip is connected to the second end of the stem.
